SEQUENCE LISTING (50 sequences)

SEQ ID NO: 1 - protein, 7 aa, SV40
PKKKRKV

SEQ ID NO: 2 - protein, 16 aa, Artificial Sequence
  Synthetic
KRPAATKKAGQAKKKK

SEQ ID NO: 3 - protein, 9 aa, Artificial Sequence
  Synthetic
PAAKRVKLD

SEQ ID NO: 4 - protein, 11 aa, Artificial Sequence
  Synthetic
RQRRNELKRSP

SEQ ID NO: 5 - protein, 38 aa, Artificial Sequence
  Synthetic
NQSSNFGPMKGGNFGGRSSGPYGGGGQYFAKPRNQGGY

SEQ ID NO: 6 - protein, 42 aa, Artificial Sequence
  Synthetic
RMRIZFKNKGKDTAELRRRRVEVSVELRKAKKDEQILKRRNV

SEQ ID NO: 7 - protein, 8 aa, Artificial Sequence
  Synthetic
VSRKRPRP

SEQ ID NO: 8 - protein, 8 aa, Artificial Sequence
  Synthetic
PPKKARED

SEQ ID NO: 9 - protein, 8 aa, Homo sapiens
PQPKKKPL

SEQ ID NO: 10 - protein, 12 aa, Mus
SALIKKKKKMAP

SEQ ID NO: 11 - protein, 5 aa, influenza NS1
DRLRR

SEQ ID NO: 12 - protein, 7 aa, influenza NS1
PKQKKRK

SEQ ID NO: 13 - protein, 10 aa, Hepatitis virus
RKLKKKIKKL

SEQ ID NO: 14 - protein, 10 aa, mus
REKKKFLKRR

SEQ ID NO: 15 - protein, 20 aa, Homo sapiens
KRKGDEVDGVDEVAKKKSKK

SEQ ID NO: 16 - protein, 17 aa, Homo sapiens
RKCLQAGMNLEARKTKK

SEQ ID NO: 17 - protein, 5 aa, Artificial Sequence
  Synthetic
GGGGS

SEQ ID NO: 18 - protein, 3 aa, Artificial Sequence
  Synthetic
  misc_feature (2)..(2): Xaa can be any naturally occurring amino acid
AXA

SEQ ID NO: 19 - protein, 15 aa, Artificial Sequence
  Synthetic
GGGGSGGGGSGGGGS

SEQ ID NO: 20 - protein, 30 aa, Artificial Sequence
  Synthetic
GGGGSGGGGSGGGGSGGGGSGGGGSGGGGS

SEQ ID NO: 21 - protein, 45 aa, Artificial Sequence
  Synthetic
GGGGSGGGGSGGGGSGGGGSGGGGSGGGGSGGGGSGGGGSGGGGS

SEQ ID NO: 22 - protein, 60 aa, Artificial Sequence
  Synthetic
GGGGSGGGGSGGGGSGGGGSGGGGSGGGGSGGGGSGGGGSGGGGSGGGGSGGGGSGGGGS

SEQ ID NO: 23 - protein, 10 aa, Artificial Sequence
  Synthetic
GGGGSGGGGS

SEQ ID NO: 24 - protein, 20 aa, Artificial Sequence
  Synthetic
GGGGSGGGGSGGGGSGGGGS

SEQ ID NO: 25 - protein, 25 aa, Artificial Sequence
  Synthetic
GGGGSGGGGSGGGGSGGGGSGGGGS

SEQ ID NO: 26 - protein, 35 aa, Artificial Sequence
  Synthetic
GGGGSGGGGSGGGGSGGGGSGGGGSGGGGSGGGGS

SEQ ID NO: 27 - protein, 40 aa, Artificial Sequence
  Synthetic
GGGGSGGGGSGGGGSGGGGSGGGGSGGGGSGGGGSGGGGS

SEQ ID NO: 28 - protein, 50 aa, Artificial Sequence
  Synthetic
GGGGSGGGGSGGGGSGGGGSGGGGSGGGGSGGGGSGGGGSGGGGSGGGGS

SEQ ID NO: 29 - protein, 55 aa, Artificial Sequence
  Synthetic
GGGGSGGGGSGGGGSGGGGSGGGGSGGGGSGGGGSGGGGSGGGGSGGGGSGGGGS

SEQ ID NO: 30 - DNA, 122 bp, Homo sapiens
gaaattaatacgactcactataggagtccgagcagaagaagaagttttagagctagaaatagcaagttaaaataaggctagtccgttatcaacttgaaaaagtggcaccgagtcggtgcttt

SEQ ID NO: 31 - DNA, 122 bp, Homo sapiens
gaaattaatacgactcactatagggtgagtgagtgtgtgcgtggttttagagctagaaatagcaagttaaaataaggctagtccgttatcaacttgaaaaagtggcaccgagtcggtgcttt

SEQ ID NO: 32 - DNA, 22 bp, Homo sapiens
gaaattaatacgactcactata

SEQ ID NO: 33 - DNA, 20 bp, Artificial Sequence
  Synthetic
taatacgactcactataggg

SEQ ID NO: 34 - DNA, 8 bp, Artificial Sequence
  Synthetic
gtcgtcgc

SEQ ID NO: 35 - DNA, 28 bp, Artificial Sequence
  Synthetic
taatacgactcactataggggtcgtcgc

SEQ ID NO: 36 - DNA, 8 bp, Artificial Sequence
  Synthetic
acgaccgc

SEQ ID NO: 37 - DNA, 28 bp, Artificial Sequence
  Synthetic
taatacgactcactatagggacgaccgc

SEQ ID NO: 38 - DNA, 8 bp, Artificial Sequence
  Synthetic
tgatgcgc

SEQ ID NO: 39 - DNA, 28 bp, Artificial Sequence
  Synthetic
taatacgactcactatagggtgatgcgc

SEQ ID NO: 40 - DNA, 8 bp, Artificial Sequence
  Synthetic
catcaatc

SEQ ID NO: 41 - DNA, 28 bp, Artificial Sequence
  Synthetic
taatacgactcactatagggcatcaatc

SEQ ID NO: 42 - DNA, 28 bp, Artificial Sequence
  Synthetic
gcgacgacccctatagtgagtcgtatta

SEQ ID NO: 43 - DNA, 28 bp, Artificial Sequence
  Synthetic
gcgacgacccctatagtgagtcgtatta

SEQ ID NO: 44 - DNA, 28 bp, Artificial Sequence
  Synthetic
gcggtcgtccctatagtgagtcgtatta

SEQ ID NO: 45 - DNA, 28 bp, Artificial Sequence
  Synthetic
gcggtcgtccctatagtgagtcgtatta

SEQ ID NO: 46 - DNA, 28 bp, Artificial Sequence
  Synthetic
gcgacgacccctatagtgagtcgtatta

SEQ ID NO: 47 - DNA, 28 bp, Artificial Sequence
  Synthetic
gcgacgacccctatagtgagtcgtatta

SEQ ID NO: 48 - DNA, 28 bp, Artificial Sequence
  Synthetic
gcggtcgtccctatagtgagtcgtatta

SEQ ID NO: 49 - DNA, 28 bp, Artificial Sequence
  Synthetic
gcggtcgtccctatagtgagtcgtatta

SEQ ID NO: 50 - DNA, 45 bp, Artificial Sequence
  Synthetic
gagtttagggataacagggtaatccttggcacccgagaattccat

(19) United States
(12) Patent Application Publication
(10) Pub. No.: US 2020/0248229 A1
(43) Pub. Date: Aug. 6, 2020

(54) UNBIASED DETECTION OF NUCLEIC ACID MODIFICATIONS

(71) Applicants: THE BROAD INSTITUTE, INC., Cambridge, MA (US); MASSACHUSETTS INSTITUTE OF TECHNOLOGY, Cambridge, MA (US)

(72) Inventors: Feng ZHANG, Cambridge, MA (US); Winston Xia YAN, Cambridge, MA (US); David Arthur SCOTT, Cambridge, MA (US)

(73) Assignees: THE BROAD INSTITUTE, INC., Cambridge, MA (US); MASSACHUSETTS INSTITUTE OF TECHNOLOGY, Cambridge, MA (US)

(21) Appl. No.: 16/310,553

(22) PCT Filed: Jun. 16, 2017
(86) PCT No.: PCT/US2017/038009

§ 371 Date: Dec. 17, 2018

Related U.S. Application Data
(60) Provisional application No. 62/351,744, filed on Jun. 17, 2016.
(60) Provisional application No. 62/377,525, filed on Aug. 19, 2016.

(51) Int. Cl. C12Q 1/6806; C12Q 1/34; C12Q 1/6855

(52) U.S. Cl. CPC C12Q 1/6806; C12Q 1/34; C12Q 1/6855; C12Q 2521/301; G01N 2333/922; C12Q 2525/186; C12Q 2525/191; C12Q 2535/122; C12Q 2565/543; C12Q 2525/155

(57) ABSTRACT

Provided herein are methods of detecting a nucleic acid modification, methods for detecting off-target activity of a targeted nuclease specific for a selected target sequence, methods for determining cleavage efficiency of a targeted nuclease specific for a selected target sequence, methods for selecting a guide RNA from a plurality of guide RNAs specific for a selected target sequence, methods for enrichment of one or more nucleic acid molecules wherein a nucleic acid modification is made and kits of parts for use in such methods.

### CROSS-REFERENCE TO RELATED APPLICATIONS

This application claims the benefit of U.S. Provisional Application No. 62/351,744 filed Jun. 17, 2016 and U.S. Provisional Application No. 62/377,525 filed Aug. 19, 2016. The entire contents of the above-identified priority applications are hereby fully incorporated herein by reference.

### STATEMENT OF GOVERNMENT SUPPORT

This invention was made with government support under grant numbers MH100706 and MH110049 awarded by the National Institutes of Health. The government has certain rights in the invention.

### TECHNICAL FIELD

The present application relates to methods and compositions for detecting nucleic acid modifications, for detecting off-target activity of a targeted nuclease, for determining cleavage efficiency of a targeted nuclease, for selecting a guide RNA from a plurality of guide RNAs and for enrichment of nucleic acid molecules wherein a nucleic acid break is made.

### BACKGROUND OF THE INVENTION

Recent advances in genome sequencing techniques and analysis methods have significantly accelerated the ability to catalog and map genetic factors associated with a diverse range of biological functions and diseases. Precise genome targeting technologies are needed to enable systematic reverse engineering of causal genetic variations by allowing selective perturbation of individual genetic elements, as well as to advance synthetic biology, biotechnological, and medical applications. Several genome-editing techniques such as designer zinc fingers, transcription activator-like effectors (TALEs), or homing meganucleases are available for producing targeted genome perturbations. The introduction of CRISPR-Cas technology to the field of genome engineering has allowed rapid and significant advances to both applications in basic science and biomedicine. Nevertheless, one of the remaining challenges is an easy and comprehensive method to capture all the off targets of the programmable endonucleases often used in genome engineering.

Numerous methods, both computational and experimental, have been devised to address this problem. In silico algorithms for predicting off targets suffer from both high numbers of false positives and false negatives, and it has been shown that numerous off-target sites predicted using these algorithms show no detectable editing when experimentally measured. Additionally, these algorithms also fail to detect multiple off targets that show experimentally validated editing. In general, available computational algorithms for off-target detection provide minimal power for off target prediction, besides providing a basic estimate of how many similar sequences exist in the genome. While in the future these may be resolved by training better algorithms based on expanding data sets for off target activity, such efforts will require comprehensive experimental validation of off-target activity in bulk using methods with much higher sensitivity and throughput than existing experimental strategies for unbiased off-target detection (GUIDEseq, BLESS, BLISS, IDLV-integration, Digenome-seq, HTGTS).

Recently, multiple experimental methods have emerged to assay off target activity in a reportedly unbiased, genome-wide manner. These include GUIDEseq, integrase deficient lentiviral (IDLV) integration, and HTGTS, Digenome-seq, and BLESS/BLISS. Although all these methods have proved very useful to elucidate the genome-wide landscape of double stranded breaks (DSBs) in different conditions, they all present a number of drawbacks. GUIDEseq, IDLV integration, and HTGTS methods require the activity of endogenous non-homologous end-joining (NHEJ) pathways to label DSBs, thus potentially missing DSBs that are not repaired via NHEJ. The formation of a translocation in HTGTS also requires the concurrent presence of two DSBs which are then ligated together for detection, which makes it a rarer event and thus lowers the detection sensitivity. Furthermore, exogenous DNA introduction in the case of GUIDEseq and IDLV integration can be challenging in primary cells and in tissues, where transfection efficiency and toxicity in the former, and delivery in the latter may be limiting factors. BLISS/BLESS are methods to directly label double stranded breaks that are not restricted by transfection efficiency and do not require NHEJ events. However, these methods are severely limited by the capture of background double strand breaks existing naturally in cells or introduced mechanically during processing. BLESS/BLISS capture a snapshot of the DSB landscape at only a single point in time, which limits their sensitivity since they do not integrate the landscape of the cutting events over time. Additionally, all of these methods occur in the context of a cell, where cellular and genomic events can influence the availability of nuclease-induced breaks to be detected.

One method to detect the off targets of Cas9 in a cell-free context is Digenome-seq, where the genomic DNA (gDNA) from a cell is extracted and in vitro digested using Cas9 and an sgRNA of interest. All of the digested gDNA is purified and prepped for next generation sequencing. The extraction of gDNA prior to digest removes all of the cellular context and focuses the determinants of Cas9 off-target activity more specifically on the thermodynamics of the interaction between the DNA, RNA, and Cas9 protein, which may be a superset of all the off targets found in a cellular context. Nevertheless, because there is no ability to enrich in the Cas9-induced breaks, whole genome sequencing (WGS) is required to get the requisite coverage for detecting the Cas9-induced cleavage sites. Despite the steady reduction in the cost of WGS, this is still an expensive proposition that additionally may result in a loss of sensitivity due the limited sequencing depth and biases in the WGS readout and mapping, such as at homopolymer sites. Without a method to specifically label the Cas9-induced breaks, these false negatives are difficult to detect and resolve. The dependence on WGS also severely limits its versatility, particularly in evaluating the cutting efficiency at on and off-target sites to determine the relative prevalence of cutting. Namely, in order to detect low-frequency editing events over the heterogeneity of background signal across the genome using WGS, the Cas9 cleavage is necessarily driven to saturation. Repeating Digenome-seq/WGS at multiple time points before saturation to evaluate relative cutting would not only be prohibitively expensive as a routing screening tool, but the low signal to noise for weak cutting events over background presents a further limitation.

Thus, there is a need for improved, unbiased and versatile methods for monitoring and detecting nucleic acid modifications. In particular, there is a need for an efficient, versatile and comprehensive method to evaluate the specificity of genome engineering technology. Further, the need exists for an unbiased, rapid, inexpensive, and high sensitivity in vitro assay for evaluating the off targets of designer endonucleases.

Citation or identification of any document in this application is not an admission that such document is available as prior art to the present invention.

### SUMMARY OF THE INVENTION

In certain example embodiments, a method for detecting a nucleic acid modification comprises contacting one or more nucleic acid molecules immobilized on a solid support with an agent capable of inducing a nucleic acid modification and sequencing at least part of said one or more immobilized nucleic acid molecules using a primer specifically binding to a primer binding site, said part comprising said nucleic acid modification. The method may comprise attaching an adapter comprising the primer binding site to one or more of the immobilized nucleic acid molecules prior to the sequencing step. The nucleic acid may be RNA or DNA and may be single or double-stranded. The immobilized nucleic acid molecules may comprise genomic DNA (gDNA) or gDNA fragments. The gDNA or gDNA fragments may be obtained from a patient in need of genome editing. The nucleic acid modification may be a methylation, a mutation, a deletion, an insertion, a replacement, a ligation, a digesting, a strand break and/or a recombination.

In certain example embodiments, the modification may be a nick, a single strand break (SSB) or a double strand break (DSB). In certain other example embodiments, the nucleic acid is double stranded, the nucleic acid modification is a nick and the method further comprises contacting said one or more immobilized nucleic acid molecules with a nuclease subsequent to said contacting with an agent capable of inducing a nick.

In certain example embodiments, the agent may comprise an chemical agent or enzyme. In certain example embodiments, the agent may be an integrase, a recombinase, a transposase, an argonaute, a cytidine deaminase, a retron and/or a group II intron. In certain example embodiments, the agent is a nuclease. The nuclease may be a targeted nuclease complex. The targeted nuclease may be a zinc finger (ZFN), a TALEN, or a CRISPR-Cas complex. The CRISPR-Cas complex may be a CRISPR-Cas II, V, or VI complex. The nuclease may comprise ca9, cas12a (cpf1), cas12b (c2c1), cas12c(c2c3), cas13a1 (c2c2), cas13a2, cas13b, and orthologs and functional equivalents thereof. In certain example embodiments, the immobilized nucleic acids may be incubated with a plurality of nucleases complexes.

The method may further comprise amplification of the one or more immobilized nucleic acids prior to contacting with an agent capable of inducing a nucleic acid modification. The method may also further comprise sequencing at least part of the immobilized nucleic acid molecules prior to the contacting step. In some embodiments, the method may further comprise comparing the sequences obtain prior to and subsequent to the contacting step.

In certain example embodiments, the immobilized nucleic acids are attached to the solid via a chemical or protein linker. The solid support may comprise a plurality of chemical or protein moieties. The method may further comprise allowing the nucleic acid molecules to be analyzed and having a first or second adapter on either end, the adapters comprising a chemical or biological moiety capable of binding to said chemical or biological moiety of said solid support, to bind to the solid support.

In certain example embodiments, the method comprises amplification of one or more nucleic acid molecules flanked by a first adapter comprising a first primer binding site and a second adapter comprising a second primer binding site in a droplet using primers specifically binding to said primer binding sites, wherein at least one other primer comprises a biological or chemical moiety capable of binding to the solid support. The amplification may comprise an emulsion amplification. In certain embodiments, the first and second adapter may hybridize to a first or second oligonucleotide immobilized on the solid support. In certain embodiments, the first adapter comprises a second that is able to hybridize to the first immobilized oligonucleotide and the second adapter comprises a sequence that is complementary to a sequence that is able to hybridize to said immobilized second oligonucleotide. The immobilized nucleic acid molecules may then be amplification, for example, using a bridge amplification.

In certain example embodiments, the method comprising allowing one or more nucleic acid molecules flanked by said first and second adapter to hybridize to a plurality of the immobilized first and second oligonucleotide, whereby the first adapter comprises a sequence that is able to hybridize to the first immobilized oligonucleotides and the second adapter hybridizes to a the second immobilized oligonucleotides. Bridge amplification is then used to amplify the immobilized target nucleic acid molecules. Bridge amplification may comprise extending said first oligonucleotide with a polymerase whereby one or more single stranded nucleic acid molecules are used as template, removing the one or more single stranded nucleic acid molecule used as template resulting in one or more single stranded immobilized nucleic acid molecules, hybridizing the one or more single stranded immobilized nucleic acid molecules to the plurality of second oligonucelotides, extending the second oligonucleotide with a polymerase resulting in double stranded immobilized nucleic acid molecules, and denaturing the double stranded immobilized nucleic acid molecules. The above steps may be repeated one or more times.

In certain example embodiments, the method for detecting off-target activity of a targeted nuclease specific for a selected target sequence comprises contacting a plurality of nucleic acid molecules immobilized on a solid support (immobilized nucleic acid molecules) with a complex comprising said targeted nuclease, thereby inducing one or more nucleic acid breaks; attaching an adapter comprising a primer binding site to one or more immobilized nucleic acid molecules comprising a nucleic acid break; sequencing at least part of said one or more immobilized nucleic acid molecules comprising a nucleic acid break using a primer specifically binding to said primer binding site; detecting the presence of breaks in a sequence of said one or more immobilized nucleic acid molecules other than in said selected target sequence.

The method for determining cleavage efficiency of a targeted nuclease specific for a selected target sequence may comprise contacting a plurality of nucleic acid molecules immobilized on a solid support (immobilized nucleic acid molecules) with a complex comprising said targeted nuclease, thereby inducing one or more nucleic acid breaks; attaching an adapter comprising a primer binding site to one or more immobilized nucleic acid molecules comprising a nucleic acid break; determining a proportion of said plurality of immobilized nucleic acid molecules comprising a nucleic acid break at said selected target sequence.

In certain example embodiments, the determining step is performed by: sequencing at least part of said one or more immobilized nucleic acid molecules comprising a nucleic acid break using a primer specifically binding to said primer binding site, or determining fluorescence intensity of said one or more immobilized nucleic acid molecules comprising said adapter which further comprises a fluorescent moiety.

The fluorescence intensity may be determined cyclically, wherein each cycle comprises addition of said complex to said plurality of nucleic acid molecules followed by determining fluorescence intensity.

In certain example embodiments, a method for selecting a guide RNA from a plurality of guide RNAs specific for a selected target sequence, comprises contacting a plurality of nucleic acid molecules immobilized on a solid support (immobilized nucleic acid molecules) with a plurality of RNA-guided nuclease complexes capable of inducing a nucleic acid break, said plurality of RNA-guided nuclease complexes comprising a plurality of different guide RNA's, thereby inducing one or more nucleic acid breaks; attaching an adapter comprising a primer binding site to said one or more immobilized nucleic acid molecules comprising a nucleic acid break; sequencing at least part of said one or more immobilized nucleic acid molecules comprising a nucleic acid break using a primer specifically binding to said primer binding site and selecting a guide RNA based on location and/or amount of said one or more breaks.

In certain example embodiments, the determining one or more locations in said one or more immobilized nucleic acid molecules may comprise a break other than a location comprising said selected target sequence and selecting a guide RNA based on said one or more locations. In certain example embodiments, the determining a number of sites in said one or more immobilized nucleic acid molecules may comprise a break other than a site comprising said selected target sequence and selecting a guide RNA based on said number of sites. The method may further comprise sequencing at least part of said one or more immobilized nucleic acid molecules prior to said contacting step. The method may further comprise comparing the sequences obtained prior to and subsequent to said contacting step. The guide RNA may be a single guide RNA (sgRNA). The nucleic acid may be RNA or DNA and single or double-stranded. The nucleic acids may comprise gDNA or gDNA fragments. In certain example embodiments, the gDNA may be obtained from a patient in need of genome editing. In certain example embodiments, the break may be a single strand break (SSB) or a double strand break (DSB).

In certain example embodiments, the agent may comprise an chemical agent or enzyme. In certain example embodiments, the agent may be an integrase, a recombinase, a transposase, an argonaute, a cytidine deaminase, a retron and/or a group II intron. In certain example embodiments, the agent is a nuclease. The nuclease may be a targeted nuclease complex. The targeted nuclease may be a zinc finger (ZFN), a TALEN, or a CRISPR-Cas complex. The CRISPR-Cas complex may be a CRISPR-Cas II, V, or VI complex. The nuclease may comprise ca9, cas12a (cpf1), cas12b (c2c1), cas12c (c2c3), cas13a1 (c2c2), cas13a2, cas13b, and orthologs and functional equivalents thereof. In certain example embodiments, the immobilized nucleic acids may be incubated with a plurality of nucleases complexes.

The method may further comprise amplification of said plurality of nucleic acid molecules prior to said contacting with said complex or said plurality of complexes. The method may further comprise sequencing at least part of said plurality of immobilized nucleic acid molecules prior to said contacting with said complex or said plurality of complexes and/or comparing the sequences obtained prior to and subsequent to said contacting with said complex or said plurality of complexes. the nucleic acid molecules may be attached to said solid support via a chemical or protein linker. The solid support may comprise a plurality of chemical or protein moieties and the method comprises, prior to said contacting step I, allowing one or more nucleic acid molecules flanked by a first and a second adapter, wherein at least one of said adapters comprises a chemical or biological moiety capable of binding to said chemical or biological moieties of said solid support to bind to said solid support.

In certain example embodiments, the method according to any one of claims may comprise, prior to the contacting step, amplifying the one or more nucleic acid molecules flanked by a first adapter comprising a first primer binding site and a second adapter comprising a second primer binding site in a droplet using primers specifically binding to said primer binding sites, wherein at least one of said primers comprises a chemical or biological moiety capable of binding to a solid support; and allowing said amplified nucleic acid molecules to bind to said solid support. The amplification may comprise emulsion amplification.

In certain example embodiments, the method may comprise allowing a plurality of nucleic acid molecules flanked by a first and a second adapter to hybridize to one of a plurality of first or second oligonucleotides that are immobilized on a solid support, whereby said first adapter comprises a sequence that is able to hybridize to said first immobilized oligonucleotides and said second adapter comprises a sequence that is complementary to a sequence that is able to hybridize to said immobilized second oligonucleotides. The method may further comprise amplifying said plurality of immobilized nucleic acid molecules. The amplifying may comprise bridge amplification. The immobilized nucleic acid molecules are unphosphorylated. In certain example embodiments, the immobilized nucleic acid molecules are treated with phosphatase prior to said contacting with said complex or said complexes. The immobilized cleaved nucleic acid molecules may be phosphorylated prior to attaching to said adapter comprising a primer binding site. The break may be a DSB and said DSB is blunt ended before attaching to said adapter comprising a primer binding site. The immobilized nucleic acid molecules may comprise a unique molecular identifier, such as a barcode. The barcode may be a DNA or RNA barcode. The solid support is selected from a chip, an array, a flow cell, a microwell, a microwell comprising an affinity treated surface and a bead, such as an immobilized affinity bead.

In another aspect, the invention provides kit of parts comprising the components for executing the methods disclosed herein. In one example embodiment, the kit of parts may comprise a solid support comprising one or more nucleic acid molecules immobilized thereon and an agent capable of inducing a nucleic acid modification. The nucleic acid modification is selected from the group consisting of a mutation, a deletion, an insertion, a replacement, a ligation, a digestion, a break and a recombination. The agent is selected from the group consisting of a chemical agent, a (viral) integrase, a recombinases, a transposase, an argonaute, a cytidine deaminase, a retron and a group II intron. The agent may comprise a targeted nuclease complex. The targeted nuclease complex may comprise a ZFN, TALEN or CRISPR-Cas. The kit may comprise a targeted nuclease and a solid support. The kit of parts may further comprise a first adapter comprising a sequence that is able to hybridize to said first immobilized oligonucleotides and a second adapter comprising a sequence that is complementary to a sequence that is able to hybridize to said immobilized second oligonucleotides. The solid support may comprise a plurality of chemical or protein linkers. The first adapter may comprise a first primer binding site and a second adapter comprising a second primer binding site, wherein at least one of said adapters comprises a chemical or biological moiety capable of binding to said chemical or protein linkers. The kit may further comprise one or more nucleic acid molecules. The nucleic acid may be RNA or DNA and single or double-stranded. The one or more nucleic acid molecules comprise gDNA or gDNA fragments. In certain example embodiments, the nucleic acid molecules are flanked by a first and a second adapter, whereby said first adapter comprises a sequence that is able to hybridize to said first immobilized oligonucleotides and said second adapter comprises a sequence that is complementary to a sequence that is able to hybridize to said immobilized second oligonucleotides. In certain example embodiments, the agent may comprise an chemical agent or enzyme. In certain example embodiments, the agent may be an integrase, a recombinase, a transposase, an argonaute, a cytidine deaminase, a retron and/or a group II intron. In certain example embodiments, the agent is a nuclease. The nuclease may be a targeted nuclease complex. The targeted nuclease may be a zinc finger (ZFN), a TALEN, or a CRISPR-Cas complex. The CRISPR-Cas complex may be a CRISPR-Cas II, V, or VI complex. The nuclease may comprise ca9, cas12a (cpf1), cas12b (c2c1), cas12c (c2c3), cas13a1 (c2c2), cas13a2, cas13b, and orthologs and functional equivalents thereof. In certain example embodiments, the immobilized nucleic acids may be incubated with a plurality of nucleases complexes.

In certain example embodiment, the of parts according may further comprise one or more components selected from the group consisting of a DNA or RNA polymerase, a restriction enzyme, a ligase, an exonuclease, a mixture of nucleotides and labelled nucleotides. The labelled nucleotides may comprise adenine, guanine, cytosine, thymine and/or uracil, whereby each nucleotide is labeled with a different fluorescent moiety. The nucleotides or labeled nucleotides may be modified nucleotides, such as dideoxy nucleotides or nucleotides comprising a phosphorothiate linkage. The solid support may be selected from a chip, an array, a flow cell, a microwell, a microwell comprising an affinity treated surface and a bead, such as an immobilized affinity bead.

In another aspect, a method for enrichment of one or more nucleic acid molecules wherein a nucleic acid modification is made, may comprise: contacting a plurality of nucleic acid molecules with an agent capable of inducing a nucleic acid modification, wherein said nucleic acid molecules are flanked by a first adapter comprising a first primer binding site and a ligation-blocking moiety and a second adapter comprising a second primer binding site and a ligation-blocking moiety, resulting in one or more modified nucleic acid molecules; and amplifying said one or more modified nucleic acid molecules comprising said adapter using a primer that binds to said first or second primer binding site and a primer that binds to a third primer binding site, wherein said method comprises attaching an adapter comprising said third primer binding site to said one or more modified nucleic acid molecules following said contacting step and prior to amplifying in step ii, or wherein said modification comprises insertion of an adapter comprising said third primer binding site.

The first and second primer binding sites may be identical. The second primer binding sites may be different. The adapter may comprise a third primer binding site and optionally a fourth primer binding site. The fourth primer binding site may be identical to said first or second primer binding site. The primer that binds to the third primer binding site may comprise a fifth primer binding site. The fifth primer binding site may be identical to said first or second primer binding site. The method may further comprise amplifying one or more nucleic acid molecules that have not been modified using said primers that bind to said first and second primer binding site.

In certain example embodiment, the plurality of nucleic acid molecules may be a plurality of RNA molecules, and said amplifying comprises reverse transcription using a primer that binds to said third primer binding site. The plurality of nucleic acid molecules may be a plurality of DNA molecules, said adapter may comprise a third primer binding that may further comprise a DNA-dependent RNA polymerase promoter and the method may further comprise, prior to said amplifying performing transcription of said one or more cleaved DNA molecules using said DNA-dependent RNA polymerase, resulting in one or more transcribed RNA molecules; and digesting DNA molecules, and wherein said amplifying may comprise amplifying said one or more transcribed RNA molecules using primers that bind to said first or second primer binding site and to said third primer binding site. The amplifying may comprise reverse transcription of said RNA molecules. The digesting may be performed using a DNAse. The primer that binds to said third primer binding site is an indexing primer.

In another example embodiment, the method for detecting a nucleic acid modification, may comprise enriching one or more nucleic acid molecules wherein a nucleic acid modification is induced according to the methods described herein, and sequencing at least part of said amplified modified nucleic acid molecules.

In another example embodiment, the method for detecting a nucleic acid modification, comprises enriching one or more nucleic acid molecules wherein a nucleic acid modification is induced with as described herein, sequencing at least part of said amplified modified nucleic acid molecules; and sequencing at least part of said amplified nucleic acid molecules that have not been modified. The adapter may comprise a first primer binding site, said adapter comprising a second primer binding site and said adapter comprising a third primer binding site are double stranded. The nucleic acid modification may be selected from the group consisting of an insertion, a replacement, a strand break and a recombination. The break may be a double stranded break (DSB), a nick or a single stranded break (SSB). The nucleic acid may be double stranded, said nucleic acid modification may a nick and the method may further comprise contacting said modified nucleic acid molecules with a nuclease subsequent to said contacting with an agent capable of inducing a nick. The break may be a double stranded break (DSB) and wherein cleaved nucleic acid molecules may be blunt ended before ligating to said adapter comprising a third primer binding site. The adapter may comprise a third primer binding site further comprises an adenine-tail. The ligation-blocking moiety may comprise a dideoxynucleotide. The adapter may comprise a first primer binding site or said adapter may comprise a second primer binding site which may further comprise a unique molecular identifier such as a barcode. The agent may comprise a nuclease. The nuclease may be a targeted nuclease complex or a plurality of targeted nuclease complexes. In certain example embodiments, the agent may comprise an chemical agent or enzyme. In certain example embodiments, the agent may be an integrase, a recombinase, a transposase, an argonaute, a cytidine deaminase, a retron and/or a group II intron. In certain example embodiments, the agent is a nuclease. The nuclease may be a targeted nuclease complex. The targeted nuclease may be a zinc finger (ZFN), a TALEN, or a CRISPR-Cas complex. The CRISPR-Cas complex may be a CRISPR-Cas II, V, or VI complex. The nuclease may comprise ca9, cas12a (cpf1), cas12b (c2c1), cas12c (c2c3), cas13a1 (c2c2), cas13a2, cas13b, and orthologs and functional equivalents thereof. In certain example embodiments, the immobilized nucleic acids may be incubated with a plurality of nucleases complexes. The one or more nucleic acid molecules comprise gDNA or fragments thereof. The gDNA or fragments thereof may be obtained from a patient in need of genome editing.

In another example embodiment, a method for detecting off-target activity of a targeted nuclease specific for a selected target sequence, may comprise: enriching one or more nucleic acid molecules wherein a nucleic acid break is induced with a method described herein, wherein said agent comprises a targeted nuclease complex and detecting the presence of breaks in a sequence of said one or more nucleic acid molecules other than in said selected target sequence.

In another example embodiment, a method for determining cleavage efficiency of a targeted nuclease specific for a selected target sequence, may comprise: enriching one or more nucleic acid molecules wherein a nucleic acid break is induced with a method as described herein, wherein said agent comprises a targeted nuclease complex and determining a proportion of said plurality of nucleic acid molecules comprising a nucleic acid break at said selected target sequence.

In another example embodiment, a method for selecting a guide RNA from a plurality of guide RNAs specific for a selected target sequence may comprise: enriching one or more nucleic acid molecules wherein one or more nucleic acid breaks are made with a method as described herein, whereby said plurality of nucleic acid molecules is contacted with a plurality of RNA-guided nuclease complexes capable of inducing a nucleic acid break; and selecting a guide RNA based on location and/or amount of said nucleic acid breaks. The selecting step may comprise determining one or more locations in said one or more nucleic acid molecules comprising a break other than a location comprising said selected target sequence and selecting a guide RNA based on said one or more locations. The selecting step may comprise determining a number of sites in said one or more nucleic acid molecules comprising a break other than a site comprising said selected target sequence and selecting a guide RNA based on said number of sites.

In another example embodiment, a method for detecting a nucleic acid break, comprises contacting a plurality of nucleic acid molecules flanked by adapters comprising a ligation-blocking moiety with an agent capable of inducing a nucleic acid break, resulting in one or more cleaved nucleic acid molecules; attaching an adapter comprising a primer binding site to said one or more cleaved nucleic acid molecules; and sequencing at least part of said one or more cleaved nucleic acid molecules using a primer specifically binding to said primer binding site, said part comprising said nucleic acid modification. In certain example embodiments, the modification comprises a beak and attaching an adapter comprising the primer binding site to said one or more immobilized nucleic acid molecules prior to sequencing. In certain example embodiments the immobilized nucleic acid molecules are unphosphorylated. In certain example embodiments, the immobilized nucleic acid molecules may be treated with a phosphatase prior to the contacting with an agent capable of inducing a modification. In certain example embodiment the one or more immobilized nucleic acid molecules comprising the nucleic acid modification are phosphorylated prior to attaching the adapter to the primer binding site. In certain example embodiments, the modification comprises a DSB and the DSB is blunt ended before attaching the adapter comprising the primer binding site. The adapter further comprising the primer binding site may further comprise a fluorescent moiety. The immobilized nucleic acid molecule may further comprise a unique molecular identifier such as a barcode. The barcode may be a DNA or RNA barcode.

In certain example embodiments, the solid support may be a chip, an array flow cell, a microwell, a microwell comprising an affinity treated surface or a bead

The following detailed description, given by way of example, but not intended to limit the invention solely to the specific embodiments described, may best be understood in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic embodiment of a method of the invention wherein genomic DNA is immobilized on a flow cell and incubated with a CRISPR-Cas9 complex. A. Initial flow cell annealing; B. Cluster amplification and sequencing of R1 and R2 reads; C. After sequencing of R1 and R2 reads, producing double stranded DNA; D. Genomic dsDNA contains Cas9 cut sites: Cas9 incubation and wash; E. Addition of a custom adapter and sequencing; and F. identifying the induced breaks.

FIG. 2 schematically shows a S1 nuclease assay, suitable for use in methods of the invention wherein the nucleic acid modification is a nick to produce cleaved double stranded DNA.

FIG. 3 shows a schematic embodiment of a method of the invention for enrichment of one or more nucleic acid molecules wherein a nucleic acid break is made wherein genomic DNA is incubated in solution with a CRISPR-Cas9 complex. A. preparation of gDNA fragments; B. preparation of gDNA substrate; C. cut with Cas9; D. Ligate 5′ adapters; E. In vitro transcription; F. DNase digest; G. reverse transcription (RT) and polymerase chain reaction (PCR).

FIGS. 4 to 6 schematically show three examples of a method of the invention for enrichment of one or more nucleic acid molecules wherein a nucleic acid modification, in particular a strand break, is made wherein genomic DNA is incubated in solution with a CRISPR-Cas9 complex.

FIG. 4 shows an example wherein first and second primer binding sites in adapters flanking the nucleic acid molecule prior to modification are identical. Nucleic acid molecules wherein a strand break is induced are selectively amplified.

FIG. 5 shows an example wherein first and second primer binding sites in adapters flanking the nucleic acid molecule prior to modification are different. Nucleic acid molecules wherein a strand break is induced are selectively amplified.

FIG. 6 shows another example wherein first and second primer binding sites in adapters flanking the nucleic acid molecule prior to modification are different and wherein, in addition to modified nucleic acid molecules, unmodified nucleic acid molecules are amplified. Such methods allows whole genome sequencing accompanying sequencing of modified nucleic acid molecules, e.g. wherein a strand break is induced.

FIG. 7 shows an example of sequencing platforms and manipulations that can be observed using certain example embodiments of the invention.

FIG. 8 shows an example of DNA manipulation on a solid surface and sequencing of these manipulations for two example sequencing platforms.

FIG. 9 is a schematic showing an in solution enrichment strategy in accordance with certain example embodiments.

FIG. 10 are asset of gels showing gDNA sample post sonication (top) and results of testing different end protection chemistries (bottom).

FIG. 11 is a gel show pre-P7 PCR gest both showing the gDNA retained post exonuclease treatment (top), a gel showing a general shift in selection of larger fragments (bottom, left) and a schematic showing the tendency of smaller amplicons to form intramolecular hairpins.

FIG. 12 is a set of gels showing results of end-blocked manipulation post CRISPR treatment.

FIG. 13 is a set of gels showing results after final library enrichment.

FIG. 14 is a set of gels showing results after manipulated DNA capture and enrichment.

FIG. 15 is a set of gels showing Cas9 versus control motif enrichment.

FIG. 16 is a schematic a schematic showing an alternative in solution enrichment strategy in accordance with certain example embodiments.

### DETAILED DESCRIPTION OF THE INVENTION

General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboraotry Manual (1988) (Harlow and Lane, eds.): Antibodies A Laboraotry Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

### Overview

The application discloses systems for direct and unbiased detection of nucleic acid modifications induced by an agent in a nucleic acid molecule fixed to a solid surface. In particular, a system is disclosed in which the on target and off target cutting of a nuclease can be assessed in a direct and unbiased way using in vitro cutting of immobilized nucleic acid molecules. This way, the superset of all cleavage targets of an targeted nuclease can be captured in an unbiased way. In addition, the invention discloses methods and systems for a genome-wide, unbiased in vitro assay that allows selective amplification of the cut fragments, thus allowing much greater sensitivity per given read over comparable in vitro methods. With these methods it is possible to enrich for and detect targeted nuclease-induced breaks, without the need for whole genome sequencing (WGS) as is required in known methods such as Digenome-seq to get the requisite coverage for detecting the nuclease-induced cleavage sites. Despite the steady reduction in the cost of WGS, this is still an expensive proposition that can result in the loss of sensitivity due the limited read depth and background reads from the uncut genome. Furthermore, biases can be introduced in both the sequencing library preparation and readout of WGS that are avoided here.

The methods of the invention make use of adapters. Preferably an adapter as used herein comprises a nucleic acid sequence, preferably a DNA sequence. In particular embodiments an adapter consists of a nucleic acid sequence and is herein also referred to as an adapter sequence. In one embodiment, the adapter sequence comprises a barcode sequence. These adapter sequences may contain sequencing primer binding sites for any next-generation sequencing technology. In one embodiment, the adapter sequences may bind one or more Illumina sequencing primers. In one embodiment, attaching an adapter comprising a primer binding site comprise ligating said adapter comprising a primer binding site to the nucleic acid molecules. In an embodiment, the methods of the invention comprise ligating a first adapter 3′ to a DNA molecule and ligating a second adapter 5′ to said DNA molecule, whereby said first adapter comprises a sequence that is able to hybridize to said first immobilized primers and said second adapter comprises a sequence that is complementary to a sequence that is able to hybridize to said immobilized second primers. In one embodiment the first (e.g. 3′) adapter comprises a T7 promoter sequence and the amplification comprises transcription by T7 polymerase. In a further embodiment, the second (e.g. 5′) adapter comprises a T5 promoter sequence and the amplification comprises transcription by T5 polymerase.

The methods of the invention are particularly suitable to test the efficiency or off-target activity of a collection of CRISPR-Cas RNA complexes in a given genomic, transcriptomic, and/or epigenomic background. A custom synthesized RNA guide can be used together with a targeted nuclease such as Cas9 or a derivative thereof, genomic DNA, and the necessary enzymes for the reaction chemistry and sequencing requirements of the method. The methods are also extremely valuable to applications in personalized medicine, and the solid support, e.g. a flow cell, could be loaded with genomic DNA from a patient to be screened. In this case, the off target assay could be run directly on the patient's genome, thus providing the most relevant information to their subsequent treatment. This has the added advantage of allowing their genome to be sequenced in the same reaction as well. In yet another embodiment, the methods and assay are used for a general purpose in vitro platform for biochemistry, in which custom DNA libraries (such as PAM libraries) are loaded onto a flow cell and subsequent cleavage reactions are performed that can be directly read out by the sequencer. From the sequencing read-out, the effector enzyme used as well as the substrate cleaved can be customized. This would minimize the hassle and inefficiency of extracting bands from a gel for library prep and NGS after an in vitro reaction, since the entire reaction can be run and read out at once.

The methods of the invention are fast with extremely high sensitivity because there is no background from spontaneous gDNA breaks or mechanically generated breaks during processing. Additionally, a single assay would provide ˜100× (NextSeq)—˜1000× (HiSeq X10) coverage of the human genome. Since the in vitro cutting of genomic DNA occurs on chip after immobilization and exponential bridge amplification of each genomic fragment, 100-1000× coverage of the human genome means 100-1000× coverage of immobilized exponentially amplified clusters covering the entire human genome. Hence, after sequencing just the ends or entirety of the DNA fragment sequences, the complete genome sequence can be determined from the genomic DNA fragments immobilized on the chip. Thereafter targeted nuclease complexes, such as Cas9 RNP complexes, may be added to the flow cell, and will cut only clusters containing off-targets for a specific RNP complex. Because the whole reaction occurs on chip, the cutting reaction can be constrained to titrate reaction sensitivity to detect both the efficiency of cutting at different off targets or run to saturation to expose all off-target sequences for a targeted nuclease, e.g. Cas9 RNP, complex with no background DSB capture, resulting in zero false positive, zero false-negative detection of Cas9 off-target activity. Only the newly cut ends by the nuclease of interest will be registered. Further, the sparse mapping of off-targets across the genome for a single RNP complex allows multiple targeted nuclease complexes targeting multiple genomic loci can to be multiplexed in a single run. It is further envisage that, multiple distinct manipulations or modifications can be performed simultaneously utilizing multiple different read-out chemistries.

In one aspect the invention provides a method for detecting a nucleic acid modification. The method can comprise: i) contacting one or more nucleic acid molecules immobilized on a solid support (immobilized nucleic acid molecules) with an agent capable of inducing a nucleic acid modification; and ii) sequencing at least part of said one or more immobilized nucleic acid molecules that comprises the nucleic acid modification using a primer specifically binding to a primer binding site. Advantageously the method comprises attaching an adapter comprising the primer binding site to the one or more immobilized nucleic acid molecules following contacting step i) and prior to sequencing step ii); alternatively or additionally, the one or more immobilized nucleic acid molecules that are contacted with the agent comprise an adapter comprising the primer binding site, e.g.: steps i) and ii) are performed wherein the one or more immobilized nucleic acid molecules that are contacted with the agent comprise an adapter comprising the primer binding site; or steps i) and ii) are performed with attaching an adapter comprising the primer binding site to the one or more immobilized nucleic acid molecules following contacting step i) and prior to sequencing step ii); or steps i) and ii) are performed with attaching an adapter comprising the primer binding site to the one or more immobilized nucleic acid molecules following contacting step i) and prior to sequencing step ii) and wherein the one or more immobilized nucleic acid molecules that are contacted with the agent comprise an adapter comprising the primer binding site.

Such methods allow for an unbiased, fast and comprehensive platform for analysis of modifications, both on-target and off-target, induced in cell-free DNA or RNA. The modifications are induced directly on nucleic acid fragments immobilized on a solid or semisolid surface, such as a sequencing platform, so that the sites of modification can be easily identified due to the nucleic acid molecules already being sequenced and registered. Because the modification is induced in the nucleic acid following library preparation, a superset of all targets is captured. The methods allow for analysis of genome-wide effects of induced modifications, in particular of genome editing applications such as targeted genome-editing nucleases. The methods are useful for a wide variety of applications, including analysis of off-target activity and efficiency of agents capable of inducing a modification, such as targeted nuclease complexes, and for selecting suitable guide RNAs specific for a selected target sequence for such targeted nuclease complexes. Such analyses are of particular high importance for therapeutic strategies involving genome-editing. For instance, the method of the invention can identify high-efficiency targeted nucleases that that manipulate a key therapeutic locus for initial therapeutic development. Further, prior to receiving therapy, the methods of the invention can be performed on a patient's own genomic DNA to analyze multiple candidate targets and to identify the target with the lowest risk for therapeutic intervention.

In a further aspect, the invention provides a method for detecting off-target activity of a targeted nuclease specific for a selected target sequence, the method comprising:
i. contacting a plurality of nucleic acid molecules immobilized on a solid support (immobilized nucleic acid molecules) with a complex comprising said targeted nuclease, thereby inducing one or more nucleic acid breaks;
ii. attaching an adapter comprising a primer binding site to one or more immobilized nucleic acid molecules comprising a nucleic acid break;
iii. sequencing at least part of said one or more immobilized nucleic acid molecules comprising a nucleic acid break using a primer specifically binding to said primer binding site;
iv. detecting the presence of breaks in a sequence of said one or more immobilized nucleic acid molecules other than in said selected target sequence.

In a further aspect the invention provides a method for determining cleavage efficiency of a targeted nuclease specific for a selected target sequence, the method comprising:
i. contacting a plurality of nucleic acid molecules immobilized on a solid support (immobilized nucleic acid molecules) with a complex comprising said targeted nuclease, thereby inducing one or more nucleic acid breaks;
ii. attaching an adapter comprising a primer binding site to one or more immobilized nucleic acid molecules comprising a nucleic acid break;
iii. determining cleavage efficiency of said plurality of immobilized nucleic acid molecules comprising a nucleic acid break at said selected target sequence.
   In particular embodiments, said determining is performed by determining a proportion of said plurality of immobilized nucleic acid molecules comprising a nucleic acid break at said selected target sequence. In particular embodiments, said determining is performed by sequencing at least part of said one or more immobilized nucleic acid molecules comprising a nucleic acid break using a primer specifically binding to said primer binding site. In particular embodiments, said determining is performed by determining a fluorescence intensity of said one or more immobilized nucleic acid molecules comprising said adapter which further comprises a fluorescent moiety. In one embodiment, said fluorescence intensity is determined cyclically, wherein each cycle comprises addition of said complex to said plurality of nucleic acid molecules followed by the step of determining fluorescence intensity.

In a further aspect, the invention provides a method for selecting a guide RNA from a plurality of guide RNAs specific for a selected target sequence, the method comprising:
i. contacting a plurality of nucleic acid molecules immobilized on a solid support (immobilized nucleic acid molecules) with a plurality of RNA-guided nuclease complexes capable of inducing a nucleic acid break, said plurality of RNA-guided nuclease complexes comprising a plurality of different guide RNA's, thereby inducing one or more nucleic acid breaks;
ii. attaching an adapter comprising a primer binding site to said one or more immobilized nucleic acid molecules comprising a nucleic acid break;
iii. sequencing at least part of said one or more immobilized nucleic acid molecules comprising a nucleic acid break using a primer specifically binding to said primer binding site.
iv. selecting a guide RNA based on location and/or amount of said one or more breaks.

In particular embodiments, step iv comprises determining one or more locations in said one or more immobilized nucleic acid molecules comprising a break other than a location comprising said selected target sequence (off-target breaks) and selecting a guide RNA based on said one or more locations. In particular embodiments, step v comprises determining a number of sites in said one or more immobilized nucleic acid molecules comprising off-target breaks and selecting a guide RNA based on said number of sites. In a further embodiment, step iv comprises both determining the location of off-targets breaks and the number of locations of off-target breaks.

In a preferred embodiment, the steps of the methods disclosed herein are performed in the indicated order.

In particular embodiments, the nucleic acid molecules are RNA molecules, such as mRNA. In other embodiments, the nucleic acid molecules are DNA molecules, such as cDNA or genomic DNA. In a particular, preferred embodiment, the nucleic acid molecules comprise genomic DNA (gDNA). In certain embodiments, the gDNA is fragmented into a plurality of smaller gDNA fragments. In particular embodiments said gDNA is obtained from a patient in need of genome editing.

In particular embodiments, the nucleic acid modification is selected from methylation, a mutation, a deletion, an insertion, a replacement, a ligation, an inversion, a digestion, a strand break and a recombination.

In particular embodiments, the agent capable of inducing a nucleic acid modification is a chemical agent. Examples of such chemical agents include, but are not limited to, etoposide and teniposide.

In particular embodiments, the agent capable of inducing a nucleic acid modification is a protein. Non-limiting examples of such proteins are a nuclease, a (viral) integrase, a recombinases, a transposase, an argonaute, a cytidine deaminase, a retron and a group II intron. In a preferred embodiment, said protein comprises a nuclease. In a particularly preferred embodiment, said agent comprises a targeted nuclease complex. Preferably the nucleic acid modification is a strand break, more preferably a SSB, a DSB or a nick, most preferably a DSB, and the agent comprises a nuclease, more preferably a targeted nuclease. In particular embodiments, said targeted nuclease complex comprises a zinc finger nuclease (ZFN), a transcription activator-like effector nuclease (TALEN) or CRISPR-Cas. In one embodiment said targeted nuclease complex comprises a RNA-directed nuclease complex. In one embodiment the targeted nuclease complex or the RNA-guided nuclease complex is a non-naturally occurring or engineered complex. In a preferred embodiment, said nuclease is selected from the group consisting of Cas9, Cpf1, C2c1, C2c2, C2c3, a group 29 nuclease, a group 30 nuclease and derivatives thereof. In a preferred embodiment, said targeted nuclease complex is a CRIPSR-Cas complex. In a particularly preferred embodiment, said CRIPSR-Cas complex comprises Cas9 or a modified Cas9.

In some embodiments, the methods comprise allowing a CRISPR complex to bind to the immobilized nucleic acid molecules to effect cleavage thereof, wherein the CRISPR complex comprises a nuclease complexed with a guide sequence hybridized or hybridizable to a target sequence within said immobilized nucleic acid molecules, wherein said guide sequence is linked to a tracr mate sequence which in turn hybridizes to a tracr sequence.

The methods provided herein allow for the simultaneous assessment of a plurality of candidate target sites as possible cleavage targets for any given nuclease, i.e. the methods of the invention are suitable for multiplexed analysis of multiple candidate target sites. Hence, in particular embodiments, the one or more immobilized nucleic acid molecules are contact with a plurality of targeted nuclease complexes, preferably with a plurality of different targeted nuclease complexes. Said plurality of targeted nuclease complexes may for instance comprises different guide RNAs specific for a single selected target sequence. Alternatively, or additionally, said plurality of targeted nuclease complexes may comprise different guide RNAs specific for different selected target sequences. Using a plurality of different targeted nuclease complexes allows for a comparison of the different complexes and selection of the most appropriate complex for the intended application. For instance, off target activity and cleavage efficiency of different targeted nuclease complexes can be assessed in a single assay. In certain example embodiments, the targeted nuclease complexes are CRISPR-Cas complexes.

In the methods of the invention one or more immobilized nucleic acid molecules are used. In a preferred embodiment, said one or more immobilized nucleic acid molecules comprise one or more clusters of immobilized nucleic acid molecules. In certain example embodiments, each cluster comprises multiple copies of a single immobilized nucleic acid molecule.

In one aspect, the invention provides a method for detecting a strand break, the method comprising:
i. sequencing at least part of one or more clusters of nucleic acid molecules immobilized on a solid support (immobilized nucleic acid molecules);
ii. contacting said immobilized nucleic acid molecules with a targeted nuclease complex, resulting in immobilized nucleic acid molecules comprising a strand break;
iii. attaching an adapter comprising a primer binding site to said immobilized nucleic acid molecules comprising a strand break; and
iv. sequencing at least part of said one or more immobilized nucleic acid molecules using a primer specifically binding to said primer binding site, said part comprising said strand break;
v. comparing the sequences obtained prior to and subsequent to said contacting with a targeted nuclease complex.

In a preferred embodiment, the method further comprises sequencing at least part of said one or more immobilized nucleic acid molecules prior to said contacting with an agent capable of inducing a nucleic acid modification. In particular embodiments, the sequences obtained prior to and subsequent to contacting the immobilized nucleic acid molecules with an agent capable of inducing a nucleic acid modification are compared for each nucleic acid molecule or for each cluster of amplified immobilized nucleic acid molecules. Comparing said sequences allows for fast detection of the presence or absence of a nucleic acid modification in the specific nucleic acid molecule. Preferably, the methods of the invention are characterized in that no amplification is carried out between the two sequencing steps.

The methods of the invention comprise sequencing at least part of nucleic acid molecules, either prior to or following modification induced by an agent as defined herein, or both. When sequencing follows agent-induced modification, said part of the nucleic acid that is sequenced preferably comprises a nucleic acid sequence of said molecule that is sufficient to allow determining whether the nucleic acid molecule has been modified, i.e. comprises an insertion, deletion, mutation, strand break, inversion etc. Said part therefore preferably comprises the nucleic acid modification, meaning that said part comprises at least the site in the sequence of the nucleic acid molecule that has been modified. In case of sequencing prior to agent induced modification as described herein, said part preferably comprises the site in the sequence of the nucleic acid molecule where the modification will be induced or is likely to be induced. The parts that are sequenced further preferably comprise one or more, such as 5, 10 or 15 nucleotides flanking the site in the sequence that has been modified or where the modification will be induced or is likely to be induced. If the site in the sequence where the modification will be induced or is likely to be induced is unknown, essentially the entire nucleic acid molecule can be sequenced. In particular embodiments, the nucleic acid molecules are sequenced, either prior to or following modification induced by an agent as defined herein, or both.

In a particular embodiment, sequencing comprises sequencing by synthesis (SBS). An SBS method can generally comprise the following steps. 1. Break up DNA into manageable fragments of about 200 to about 600 base pairs. 2. Short sequences of DNA called adaptors, are attached to the DNA fragments. 3. The DNA fragments attached to adaptors are then made single stranded. This can be done by incubating the fragments with a base such as sodium hydroxide. 4. Once prepared, the DNA fragments are washed across a flowcell. The complementary DNA binds to primers on the surface of the flowcell and DNA that does not attach is washed away. 5. The DNA attached to the flowcell is then replicated to form small clusters of DNA with the same sequence. When sequenced, each cluster of DNA molecules will emit a signal that is strong enough to be detected by a camera. 6. Unlabeled nucleotide bases and DNA polymerase are then added to lengthen and join the strands of DNA attached to the flowcell. This creates ‘bridges’ of double-stranded DNA between the primers on the flow cell surface. 7. The double-stranded DNA is then broken down into single-stranded DNA using heat, leaving several million dense clusters of identical DNA sequences. 8. Primers and fluorescently-labelled terminators (terminators are a version of nucleotide base—A, C, G or T—that stop DNA synthesis) are added to the flow cell. 9. The primer attaches to the DNA being sequenced. 10. The DNA polymerase then binds to the primer and adds the first fluorescently-labelled terminator to the new DNA strand. Once a base has been added no more bases can be added to the strand of DNA until the terminator base is cut from the DNA. 11. Lasers are passed over the flowcell to activate the fluorescent label on the nucleotide base. This fluorescence is detected by a camera and recorded on a computer. Each of the terminator bases (A, C, G and T) give off a different color. 12. The fluorescently-labelled terminator group is then removed from the first base and the next fluorescently-labelled terminator base can be added alongside. And the process continues until a large number, e.g., millions of clusters have been sequenced. 13. The DNA sequence is analysed base-by-base during this sequencing, making it a highly accurate method. The sequence generated can then be aligned to a reference sequence, this looks for matches or changes in the sequenced DNA. SBS may include Next Generation Sequencing (NGS) and high throughput forms of SBS. In particular embodiments, sequencing comprises NGS, also referred to as high-throughput sequencing. Technologies for NGS are known in the art, examples include Illumina (Solexa) sequencing, Roche 454 sequencing, Ion torrent: Proton/PGM sequencing, and SOLiD sequencing.

In particular embodiments, the methods of the invention comprise amplification of said one or more immobilized nucleic acid molecules prior to said contacting with an agent capable of inducing a nucleic acid modification. Thereby, a plurality of immobilized nucleic acid molecules is produced. Such plurality of immobilized nucleic acid molecules resulting from amplification is herein also referred to as amplified immobilized nucleic acid molecules or a cluster of amplified immobilized nucleic acid molecules. Said amplification is preferably performed prior to contacting the immobilized nucleic acid molecules with an agent capable of inducing a nucleic acid modification so that potential bias resulting from such amplification is avoided.

Indeed, one of the main advantages of the methods of the invention is that essentially no manipulation (apart from converting single stranded nucleic acid molecules into double stranded nucleic acid molecules or vice versa) such as amplification is performed with the nucleic acid molecules after contacting with the agent capable of inducing a modification, which manipulations could introduce bias. Hence, in a preferred embodiment, the method does not comprise an amplification step after immobilized nucleic acid molecules have been contacted with an agent capable of inducing a nucleic acid modification such as a targeted nuclease complex and prior to sequencing said immobilized nucleic acid molecules thereafter.

In particular embodiments said amplifying comprises bridge amplification. In particular embodiments, the methods of the invention comprise:

a) allowing one or more nucleic acid molecules flanked by said first and second adapter to hybridize to one of said plurality of first or second immobilized oligonucleotides, whereby said first adapter comprises a sequence that is able to hybridize to said first immobilized oligonucleotides and said second adapter comprises a sequence that is complementary to a sequence that is able to hybridize to said immobilized second oligonucleotides;

and the bridge amplification comprises:

b) extending said first oligonucleotide with a polymerase whereby said one or more single stranded nucleic acid molecules flanked by a first and a second adapter are used as a template;

c) removing said one or more single stranded nucleic acid molecules flanked by a first and a second adapter used as a template resulting in one or more single stranded immobilized nucleic acid molecules;

d) hybridizing said one or more single stranded immobilized nucleic acid molecules to one of said plurality of immobilized second oligonucleotides;

e) extending said second oligonucleotide with a polymerase resulting in one or more double stranded immobilized nucleic acid molecules;

f) denaturing said one or more double stranded immobilized nucleic acid molecules to produce a plurality of immobilized single stranded nucleic acid molecules; and

g) repeating steps d-f at least once.

Steps d-f are preferably repeated multiple times so that a cluster of identical nucleic acid molecules is obtained for each of the plurality of nucleic acid molecules. Said cluster preferably comprises sufficient nucleic acid molecules to allow sequencing. Each cluster may contain for instance one million copies of the original nucleic acid molecule.

In particular embodiments, methods of the invention for detecting off target activity of a targeted nuclease specific for a selected target sequence, for determining cleavage efficiency of a targeted nuclease specific for a selected target sequence or for selecting a guide RNA from a plurality of guide RNAs specific for a selected target sequence further comprise:

a) allowing one or more nucleic acid molecules flanked by a first and a second adapter to hybridize to one of said plurality of first or second immobilized oligonucleotides, whereby said first adapter comprises a sequence that is able to hybridize to said first immobilized oligonucleotides and said second adapter comprises a sequence that is complementary to a sequence that is able to hybridize to said immobilized second oligonucleotides;

and the bridge amplification comprises:

b) extending said first oligonucleotide with a polymerase whereby said single stranded nucleic acid molecules flanked by a first and a second adapter are used as a template;

c) removing said single stranded nucleic acid molecules flanked by a first and a second adapter used as a template resulting in a plurality of single stranded immobilized nucleic acid molecules;

d) hybridizing said one or more single stranded immobilized nucleic acid molecules to one of said plurality of immobilized second oligonucleotides;

e) extending said second oligonucleotide with a polymerase resulting in a plurality of double stranded immobilized nucleic acid molecules;

f) denaturing said plurality of double stranded immobilized nucleic acid molecules to produce a plurality of immobilized single stranded nucleic acid molecules; and

g) repeating steps d-f at least once.

Steps d-f are preferably repeated multiple times so that a cluster of identical nucleic acid molecules is obtained for each of the plurality of nucleic acid molecules. Said cluster preferably comprises sufficient nucleic acid molecules to allow sequencing. Each cluster may contain for instance one million copies of the original nucleic acid molecule.

In particular embodiments, the nucleic acid molecules are attached to said solid support via a chemical or protein linker.

In particular embodiments, the solid support comprises clusters of immobilized nucleic acids. In some embodiments, nucleic acid molecules are amplified prior to immobilization on the solid support. For instance, amplification prior to immobilization comprises emulsion amplification, which is particularly suitable to obtain clusters of nucleic acid molecules comprising multiple copies of the same original nucleic acid molecule. In particular embodiments, a solid support comprising a plurality of chemical or protein moieties is used in a method of the invention and the method comprises, prior to the contacting step i, allowing one or more nucleic acid molecules flanked by a first and a second adapter, wherein at least one of the adapters comprises a chemical or biological moiety capable of binding to said chemical or biological moieties of said solid support, to bind to said solid support. In particular embodiments the methods of the invention method comprises prior to said contacting step i: amplification of one or more nucleic acid molecules flanked by a first adapter comprising a first primer binding site and a second adapter comprising a second primer binding site in a droplet using primers specifically binding to said primer binding sites, wherein at least one of said primers comprises a chemical or biological moiety capable of binding to a solid support; and allowing said amplified nucleic acid molecules to bind to said solid support.

The methods of the invention use an adapter comprising a primer binding site. Said primer binding site is preferably used in amplification and/or sequencing of the immobilized nucleic acid molecules. For instance, the immobilized nucleic acid molecule (prior to modification) may be flanked on either end of the fragment by adapters comprising a different primer binding site. These primer binding sites can be used to amplify the immobilized nucleic acid molecules prior to contacting with the modification inducing agent, for instance by bridge amplification as described herein elsewhere. Further, if one of the adapters comprising a primer binding site is removed as a result of the nucleic acid modification induced by the agent, such as a strand break, another, third, adapter can be attached to the modified nucleic acid molecules, for instance at the site of the strand break. Such attachment is optionally executed after blunt ending of modified nucleic acid molecules. Such third adapter an be used for sequencing of the nucleic acid molecules. In particular embodiments of the invention only nucleic acid molecules wherein a modification such as a strand break is induced are sequenced. Thus, in preferred embodiment, nucleic acid molecules wherein a modification such as a strand break is induced are selectively sequenced. This is for instance achieved by using an adapter for attaching to modified, e.g. cleaved, nucleic acid molecules that is distinguishable from the adapters flanking the nucleic acid molecules prior to inducing the modification. In particular embodiments of the invention nucleic acid molecules are sequenced both prior to and following modification. A comparison of the sequences of the same nucleic acid molecules prior to and following inducing the modification can be made.

In particular embodiments, a method of the invention for detecting a nucleic acid modification comprises attaching an adapter comprising the primer binding site to said one or more immobilized nucleic acid molecules following step i and prior to sequencing in step ii. Such method is particularly suitable if the nucleic acid modification comprises a DSB, a SSB or a nick, which results in cleavage of the immobilized nucleic acid molecules. In that case an adapter comprising the primer binding site already present on the immobilized nucleic acid molecules is potentially cleaved of, resulting in nucleic acid molecules lacking a primer binding site. Hence, in a method wherein said nucleic acid modification comprises a strand bread, the adapter comprising the primer binding site is preferably attached to the nucleic acid molecule after contacting with the agent capable of inducing the strand break.

In particular embodiments, in such methods the adapter comprising the primer binding site is specific for the immobilized nucleic acid molecules that have been cleaved. That way the adapter is only attached to the immobilized nucleic acid molecules wherein a strand break is induced and not to unmodified nucleic acid molecules. This is for instance achieved by phosphatase treatment of the immobilized nucleic acid molecules prior to contacting with the agent capable of inducing a nucleic acid modification, in particular a strand break. Hence, in particular embodiments, one or more immobilized nucleic acid molecules are unphosphorylated. Further, the one or more immobilized nucleic acid molecules comprising a nucleic acid modification, preferably a strand break, are preferably phosphorylated prior to attaching to said adapter comprising a primer binding site.

In particular embodiments, wherein said nucleic acid modification comprises a DSB which results in an overhang, said DSB is blunt ended before attaching to said adapter comprising a primer binding site.

In particular embodiments, in a method of the invention for detecting a nucleic acid modification the one or more immobilized nucleic acid molecules that are contacted with said agent already comprise an adapter comprising said primer binding site. Such method is particularly suitable if the nucleic acid modification does not result in a strand break or cleavage of the nucleic acid molecules. Such nucleic acid modifications for instance comprise an insertion, a deletion, a substitution or a rearrangement. Alternatively, the adapter comprising the primer binding site is attached to the nucleic acid molecules after contacting with the agent capable of inducing such modification. In both alternatives, the primer binding site will be available for use in sequencing the immobilized nucleic acid molecules.

In particular embodiments, the adapter comprising the primer binding site comprises a modified nucleic acid, a chemical moiety, an affinity moiety, or a fluorescent moiety. Such modified nucleic acid or chemical, affinity or fluorescent moiety allows for detection of immobilized nucleic acid molecules other than by sequencing. In such methods, the nucleic acid modification preferably comprises a strand break and the adapter is attached to the immobilized nucleic acid molecules after contacting with the agent capable of inducing the strand break.

In particular embodiments, the solid support is selected from a chip, an array a flow cell, a microwell, a microwell comprising an affinity treated surface and a bead, such as an immobilized affinity bead. In a preferred embodiment, the solid support is a flow cell or a bead, more preferably a flow cell.

In particular embodiments, the immobilized nucleic acid molecules comprise a “unique molecular identifier” (UMI). The term “UMI” refers to a sequencer linker used in a method that uses molecular tags to detect and quantify unique amplified products. In the method of the present invention, a UMI may be used to distinguish nucleic acid molecules wherein a modification, such as a break, has been induced from nucleic acid molecules not containing said nucleic acid modification. Alternatively, they may be used to distinguish nucleic acid molecules wherein a modification, such as a break, has been induced in a selected target sequence from nucleic acid molecules wherein the modification has been induced in a sequence of said one or more immobilized nucleic acid molecules other than in said selected target sequence.

In particular embodiments, the one or more immobilized nucleic acid molecules comprise a barcode, preferably a DNA barcode if the nucleic acid is DNA or an RNA barcode if the nucleic acid is RNA. The term barcode as used herein, refers to any unique, non-naturally occurring, nucleic acid sequence that may be used to identify the originating genome of a nucleic acid fragment.

In one aspect, the invention provides a kit of parts for executing a method according to the invention.

In one aspect, the invention provides a kit of parts comprising a solid support comprising one or more nucleic acid molecules immobilized thereon and an agent capable of inducing a nucleic acid modification. Such kit of parts is particularly suitable for detecting nucleic acid modifications in nucleic acid molecules, such as genomic DNA, in accordance with the methods of the invention.

In particular embodiments, the nucleic acid modification is selected from methylation, a mutation, a deletion, an insertion, a replacement, a ligation, a digestion, a strand break and a recombination. In particular embodiments, the agent of said kit of parts is selected from a nuclease, a chemical agent, a (viral) integrase, a recombinases, a transposase, an argonaute, a cytidine deaminase, a retron and a group II intron. Preferably the nucleic acid modification is a strand break, more preferably a SSB, a DSB or a nick and the agent in said kit of parts comprises a nuclease, more preferably a targeted nuclease. In particular embodiments, the agent comprises a targeted nuclease complex. In particular embodiments, said complex comprises a ZFN, TALEN or CRISPR-Cas. In a preferred embodiment, said nuclease is selected from the group consisting of Cas9, Cpf1, C2c1, C2c2, C2c3, a group 29 nuclease, a group 30 nuclease and derivatives thereof. In a preferred embodiment, said targeted nuclease complex is a CRIPSR-Cas complex.

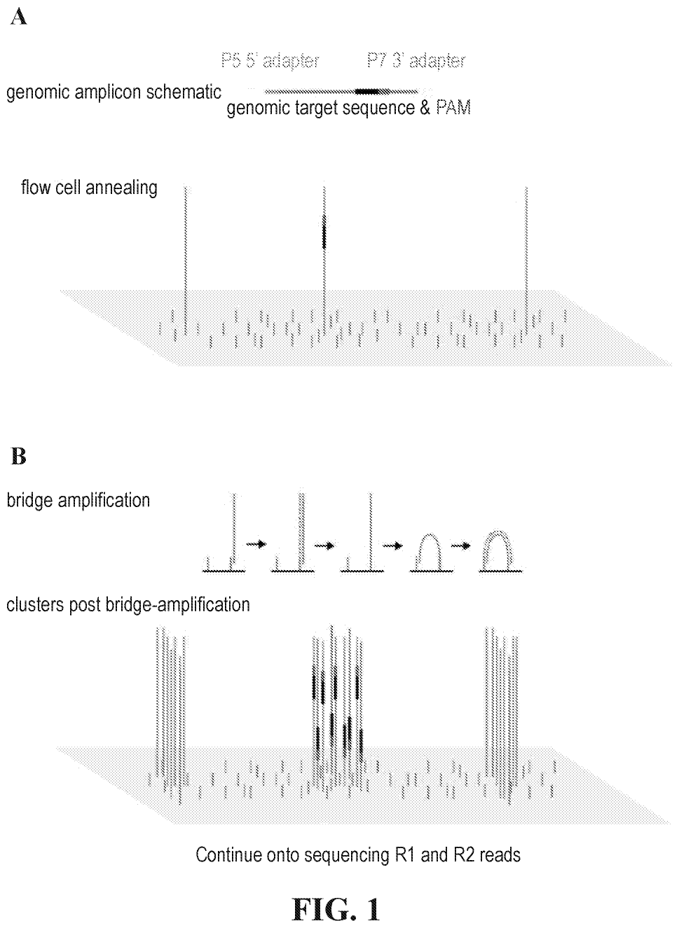

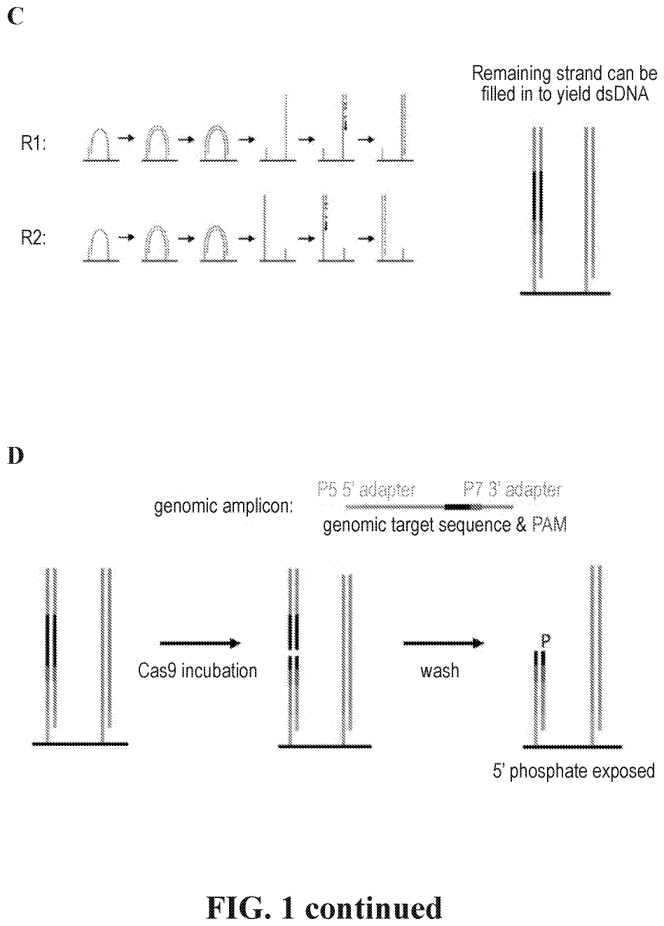

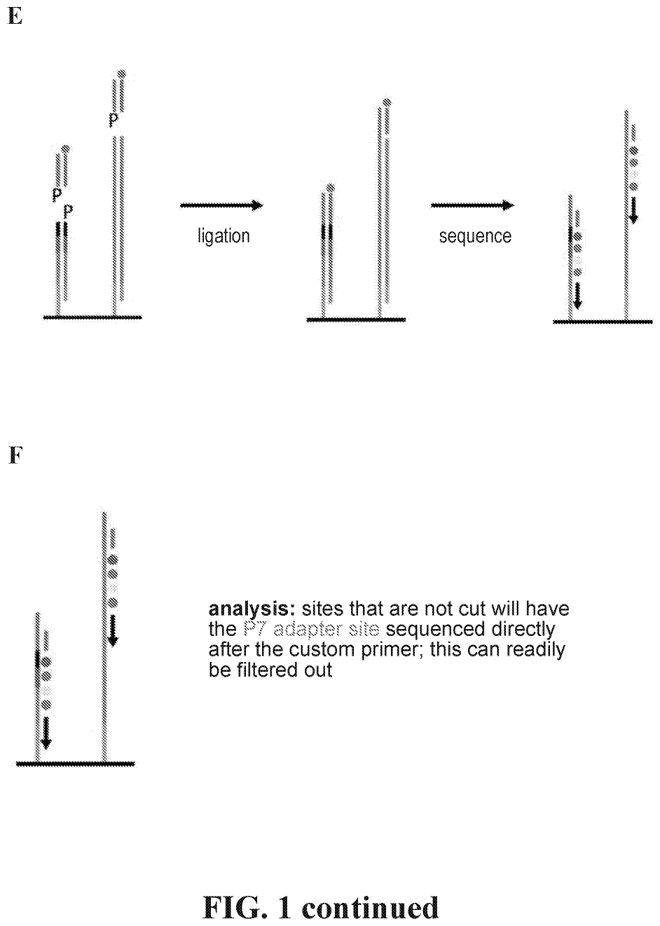

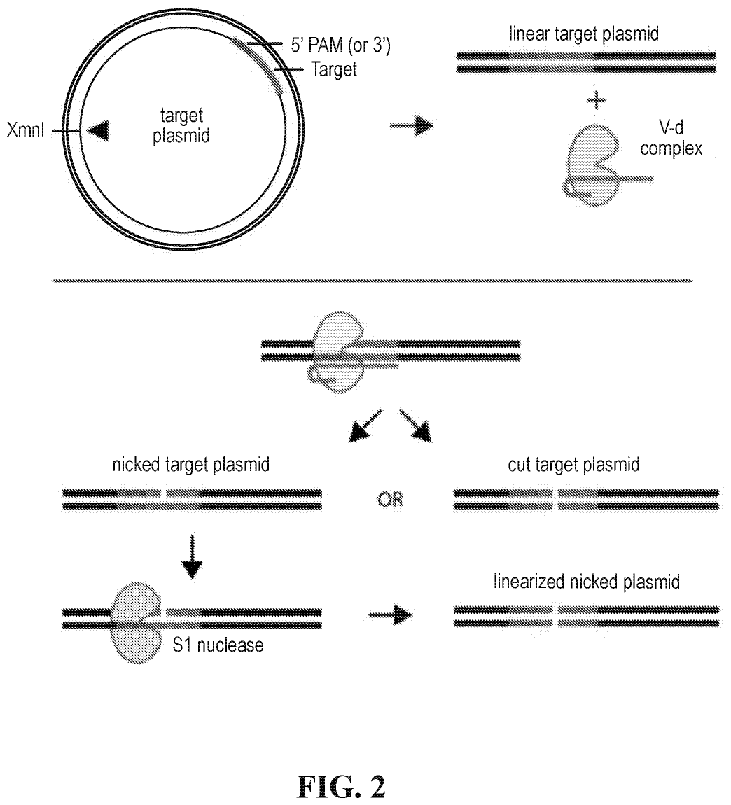

In certain example embodiments, a genomic DNA library is prepared and the gDNA fragments are provided with an adapter comprising a primer binding site on each end, referred as the first and second adapter. For instance, the first adapter is a P5 adapter and is attached 5′ of the fragments and the second adapter is a P7 adapter and is attached 3′ of the fragments. The adapter-flanked fragment are subsequently annealed to a flow cell comprising oligo's that are able to hybridize to the adapter sequences. Bridge amplification, described herein elsewhere, is performed to provide clusters of amplified nucleic acids for each gDNA fragment annealed to the flow cell. One or both of the strands of the resulting amplified double stranded DNA are subsequently sequenced, for instance using SBS, referred to as the R1 and R2 reads. Following sequencing, the remaining strand is converted into double stranded DNA. The amplified nucleic acid sequences are then contacted with the modification agent. In certain example embodiments, the modification agent results in a DSB in the nucleic acid fragments. The nucleic acid fragments with DSBs are then labeled attached at one end via an adapter to the flow cell and are unlabeled on a new terminal end generated at the site of the DSB. A third adapter comprising a primer binding site may then be added to the new terminal end of the modified nucleic acid fragments. In certain embodiments, the third adapter will also be added to the unmodified nucleic acid fragments, i.e. to either the first or second adapter that is not attached to the flow cell. In certain embodiments, the third adapter is selectively attached to modified nucleic acid fragments only and not to unmodified fragments. Sequencing of the modified and optionally unmodified sequences is then carried out to analyze DSBs and optionally to determine if any off target modification sites were generated by the modification agent. Either only the modified fragments or both the modified and unmodified fragments may be sequenced using a primer to the third adapter. In the unmodified fragments, the third adapter is attached directly to the first or second adapter which is readily detected when sequencing, allowing direct filtering out of the unmodified sequences. The modified nucleic acids can be analyzed to determine cleavage sites in the genome. FIG. 1 provides a schematic overview of such method.

In one aspect, the invention provides a kit of parts comprising a targeted nuclease and a solid support. Such kit of parts is particularly suitable for detecting nucleic acid modifications in nucleic acid molecules, such as genomic DNA, in accordance with the methods of the invention. Advantageously, said solid support is configured to allow attachment of nucleic acid molecules. Hence, in particular embodiments, the solid support comprises a plurality of first and second oligonucleotides immobilized thereto. In particular embodiments, the kit of parts further comprises a first adapter comprising a sequence that is able to hybridize to said first immobilized oligonucleotides and a second adapter comprising a sequence that is complementary to a sequence that is able to hybridize to said immobilized second oligonucleotides.

Alternatively, the kit of parts comprises a solid support comprising a plurality of chemical or protein linkers. Such linkers can be used to bind nucleic acid molecules functionalized with a chemical or protein moiety able to bind to such linkers. Hence, in particular embodiments, such kit of parts further comprises a first adapter comprising a first primer binding site and a second adapter comprising a second primer binding site, wherein at least one of said adapters comprises a chemical or biological moiety capable of binding to said chemical or protein linkers.

Such kit of parts comprising a solid support configured to allow attachment of nucleic acid molecules are particularly suitable for detecting nucleic acid modifications in nucleic acid obtained from a patient, such as a patient in need of genomic editing. Indeed, the genomic DNA of a patient can be fragmented, followed by attachment of the first and second adapter to the genomic DNA fragments on both ends of the fragments. Subsequently the genomic DNA fragments flanked by the first and second adapter can be immobilized to the solid support by attaching to the plurality of first or second oligonucleotides immobilized thereon. Method to immobilize nucleic acid fragments to the solid support are described herein below.

In particular embodiments, the kit of parts comprising a targeted nuclease and a solid support comprising a plurality of first and second oligonucleotides immobilized thereto further comprises one or more nucleic acid molecules. In particular embodiments, said nucleic acid molecule are flanked by a first and a second adapter, whereby said first adapter comprises a sequence that is able to hybridize to said first immobilized oligonucleotides and said second adapter comprises a sequence that is complementary to a sequence that is able to hybridize to said immobilized second oligonucleotides.

In particular embodiments, the nucleic acid molecules comprised in a kit of part according to the invention comprise RNA. In particular embodiments, the nucleic acid molecules comprised in a kit of part according to the invention comprise DNA. In particular embodiments, the nucleic acid molecules comprised in a kit of part according to the invention are double stranded. In particular embodiments, the nucleic acid molecules comprised in a kit of part according to the invention are single stranded. In a particular, preferred, embodiment, the nucleic acid molecules comprise genomic DNA (gDNA). Preferably, said nucleic acid molecules comprising gDNA comprise gDNA fragments.

A kit of part of the invention may further contain one or more of the components such as the primers and enzymes necessary for the reaction chemistry and sequencing performed in the assay's of the invention. Hence, in particular embodiments, a kit of parts according to the invention further comprises one or more components selected from the group consisting of one or more primers, a DNA or RNA polymerase, a restriction enzyme, a ligase, an exonuclease, a mixture of nucleotides and labelled nucleotides. Said labelled nucleotides are for instance adenine, guanine, cytosine, thymine and/or uracil, whereby each nucleotide is labelled with a different fluorescent moiety. Such fluorescently labelled nucleotides are particularly suitable for sequencing by synthesis as explained in more detail herein below.

The nucleotides encompasses within a kit of parts of the invention are suitably modified, for instance modulation of ligation and nucleotide manipulation purposes. Hence, in particular embodiments, the nucleotides or fluorescently labeled nucleotides are modified nucleotides. Non-limiting examples of modified nucleotides are dideoxy nucleotides or nucleotides comprising a phosphorothiate linkage. Dideoxynucleotides (also referred to as 2′,3′ dideoxynucleotides) are chain-elongating inhibitors of DNA polymerase and block ligation of further polynucleotides. They are abbreviated as ddNTPs (ddGTP, ddATP, ddTTP and ddCTP). The absence of the 3′-hydroxyl group means that, no further nucleotides can be added as no phosphodiester bond can be created based on the fact that deoxyribonucleoside triphosphates allow DNA chain synthesis or ligation to occur through a condensation reaction between the 5′ phosphate (following the cleavage of pyrophospate) of the current nucleotide with the 3′ hydroxyl group of the previous nucleotide. A phosphorothioate linkage substitutes a sulfur atom for a non-bridging oxygen in the phosphate backbone of an polynucleotide. This modification renders the internucleotide linkage resistant to nuclease degradation. Phosphorothioate linkages can typically be introduced between the last 3-5 nucleotides at the 5′- or 3′-end of the polynucleotide.

In particular embodiments, the solid support encompassed in a kit of parts of the invention is selected from a chip, an array a flow cell, a microwell, a microwell comprising an affinity treated surface and a bead, such as an immobilized affinity bead. In a preferred embodiment, the solid support is a flow cell or a bead, more preferably a flow cell or a bead.

In one aspect the invention provides method for enrichment of one or more nucleic acid molecules wherein a nucleic acid modification is made, the method comprising contacting a plurality of nucleic acid molecules with an agent capable of inducing a nucleic acid modification, wherein said nucleic acid molecules are flanked by a first adapter comprising a first primer binding site and a ligation-blocking moiety and a second adapter comprising a second primer binding site and a ligation-blocking moiety, resulting in one or more modified nucleic acid molecules; and amplifying said one or more cleaved nucleic acid molecules comprising said adapter using a primer that binds to said first or second primer binding site and a primer that binds to a third primer binding site. Advantageously the method comprises attaching an adapter comprising the third primer binding site to said one or more modified nucleic acid molecules following the contacting step and prior to amplifying in step ii); alternatively or additionally, the modification comprises insertion of an adapter comprising the third primer binding site, e.g.: steps i) and ii) are performed wherein the modification comprises insertion of an adapter comprising the third primer binding site; or steps i) and ii) are performed with attaching an adapter comprising the third primer binding site to the one or more modified nucleic acid molecules following said contacting step and prior to amplifying in step ii); or steps i) and ii) are performed with attaching an adapter comprising the third primer binding site to the one or more modified nucleic acid molecules following the contacting step and prior to amplifying in step ii)) and wherein the modification comprises insertion of an adapter comprising the third primer binding site. Advantageously said method is performed in solution.

Such method allows for specific enrichment of nucleic acid molecules that have been modified, for instance by inducing a strand break, in vitro. Such methods are highly sensitive. In addition, they can easily be multiplexed, i.e. the methods allo for enrichment of multiple modification is possible in a single assay. The methods for enrichment of nucleic acid molecules wherein a nucleic acid modification has been made are particularly suitable for use in methods and assays for determining off target activity or cleavage efficiency of targeted (endo)nucleases in genomic DNA in solution. Enrichment of modified, e.g. cleaved, nucleic acid molecules avoids the need to perform whole genome sequencing in order to monitor and analyze targeted nuclease activity. Such whole genome sequencing is necessary in the only in vitro off-target detection assay that is currently available, i.e. Digenome-seq. The enrichment methods of the invention, followed by detection of strand breaks are particularly suitable for use as a control in cell-based off target assay's such as BLISS/BLESS.

In particular embodiments, the first and second primer binding sites are identical. This is for instance achieved if the first and second adapter are identical. Such methods are particularly suitable for enrichment and specific sequencing of modified nucleic acids, without sequencing unmodified nucleic acids. In other embodiments, the first and second primer binding sites are different. This is for instance achieved if the first and second adapter are different. Such method provides at least two possibilities for further processing: a first one for enrichment and specific sequencing of modified nucleic acids, without sequencing unmodified nucleic acids, and a second one for enrichment and sequencing of both modified and unmodified nucleic acids. Therefore, in particular embodiments wherein said first and second primer binding sites are different, the adapter comprising a third primer binding site further comprises a fourth primer binding site, which may be identical to the first or second primer binding site. In other embodiments wherein said first and second primer binding sites are different, the primer that binds to the third primer binding site comprises a fifth primer binding site, which may be identical to the first or second primer binding site. Amplification with such primer creates an overhang with an additional primer binding site.

In certain example embodiments, the nucleic acids to be tested are fragmented using fragmentation methods known in the art and discussed elsewhere herein to yield a plurality of smaller nucleic acid fragments. One or more smaller nucleic acid fragments may comprise modification target sites/sequences. In certain example embodiments, the test nucleic acid to be fragmented is genomic DNA. Each resulting fragmented nucleic strand is then labeled on each terminal end with an adapter. The adapter comprises a primer binding site. In one example embodiment, each nucleic acid fragment is labeled with the same adapter on each terminal end. In another example embodiment, each nucleic acid fragment is labeled on a first terminal end with a first adapter and on the second terminal end with a second adapter. The fragmented nucleic acids are then amplified.

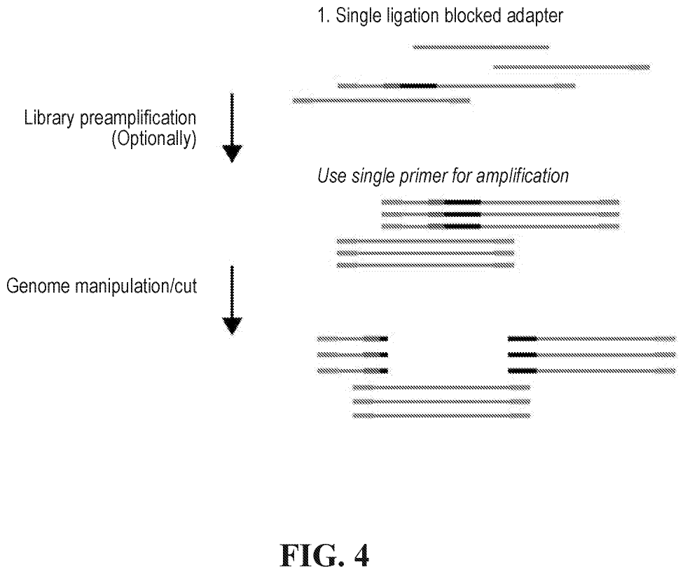

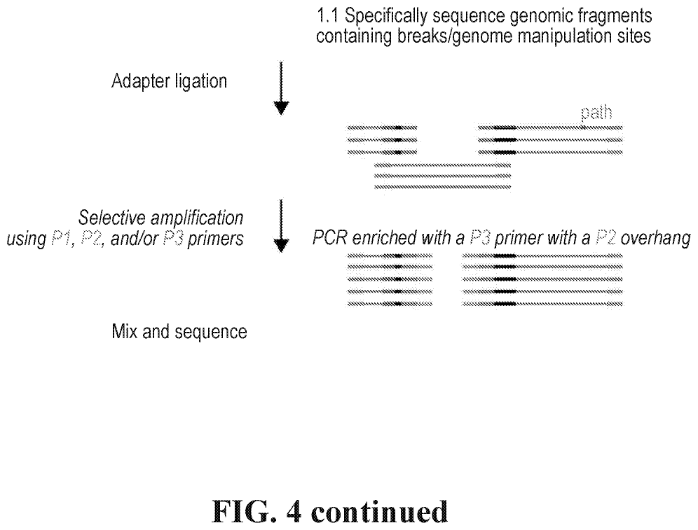

In embodiments where the nucleic acid fragments are labeled with the same first adapter on both terminal ends—each adapter comprising the same primer binding site—a single primer may be used to amplify the nucleic acid fragments. Thus, both nucleic acid fragments comprising a modification target site/sequence and nucleic acid fragments that do not comprise a modification target site/sequence can be amplified. The amplified nucleic acid sequences are then contacted with the modification agent. In certain embodiments, the modification agent results in a double-stranded break in nucleic acid fragments. Those nucleic acid fragments with DSBs are then labeled one end with the original adapter and unlabeled on a new terminal end generated at the site of the DSB. A second adapter comprising a primer binding site may then be added to the new terminal end of the modified nucleic acid fragments. The modified nucleic acid fragments may then be enriched by amplification using a pair of primers corresponding to the primer binding sites in the first and second adapters. In certain example embodiments, a further, third, adapter may then be ligated to the second adapter for purposes of sequencing. For example, if the first adapter is a P7 adapter, the third adapter may be a P5 adapter to enable sequencing by synthesis (SBS) of the modified nucleic acid fragments. Sequencing of the modified nucleic acids is for instance carried out to analyze DSBs and optionally to determine if any off target modification sites were generated by the modification agent. FIG. 4 provides a schematic overview of an example of en enrichment method as described herein wherein first and second primer binding sites that are part of adapter flanking the nucleic acid molecule are identical.

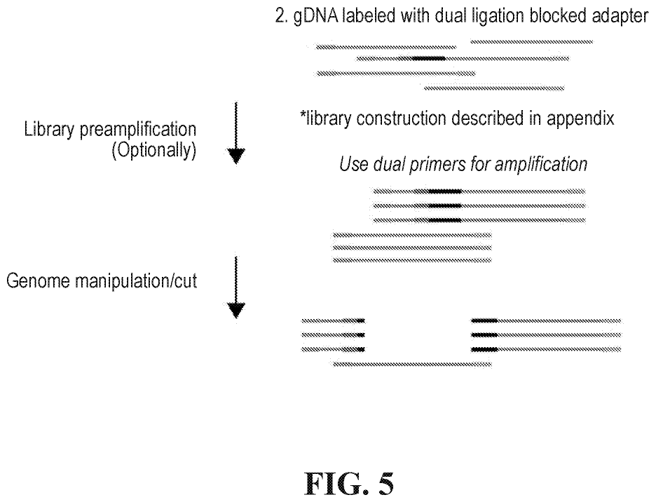

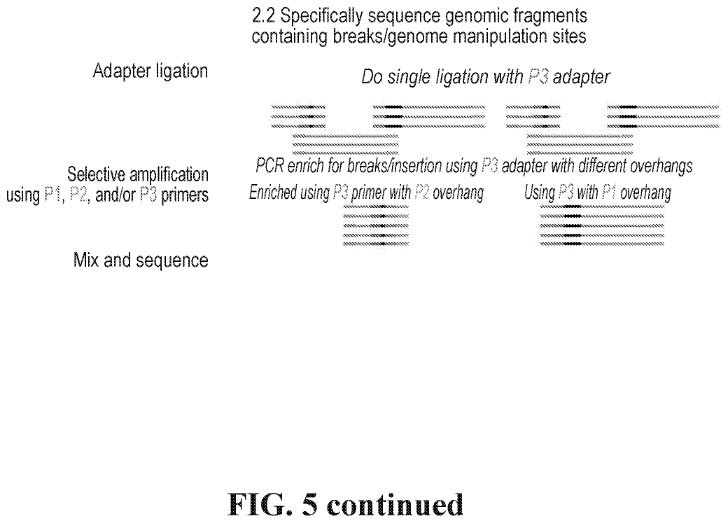

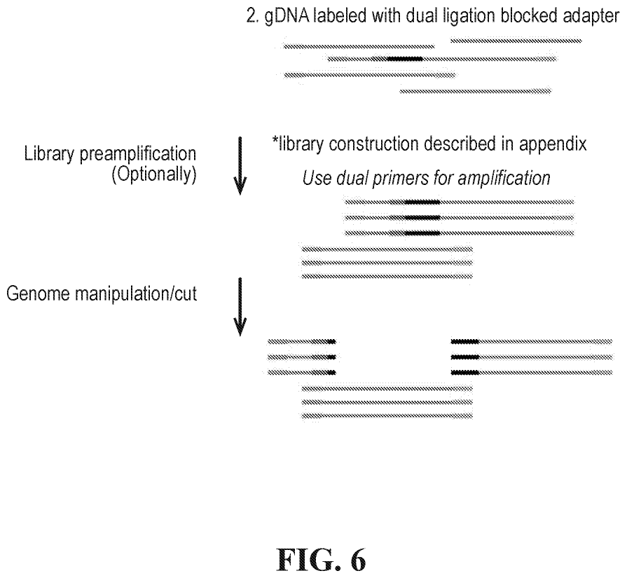

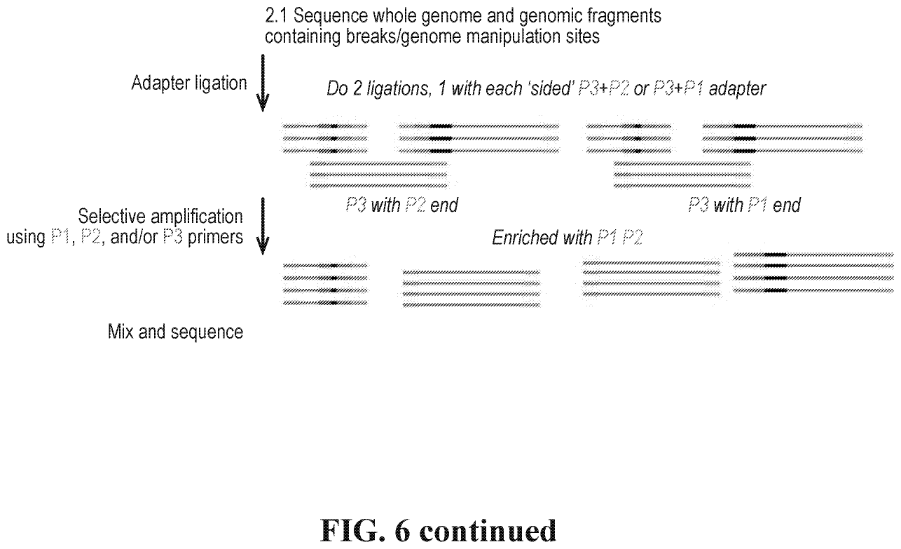

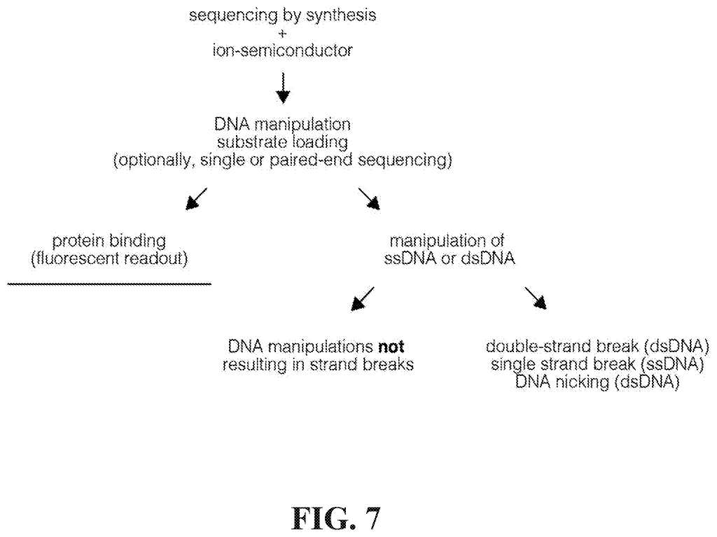

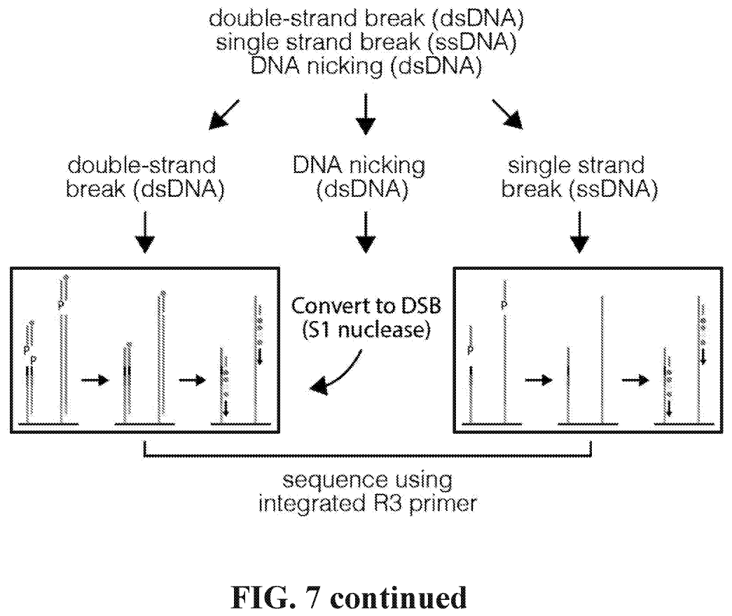

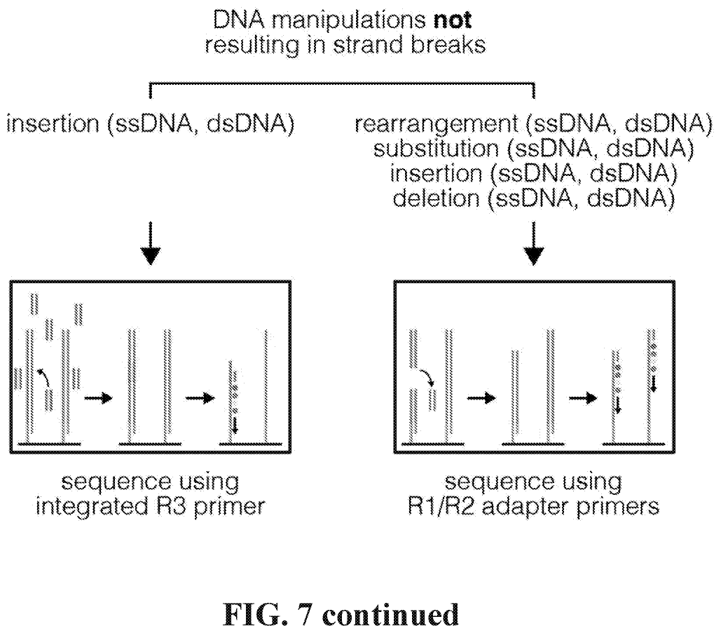

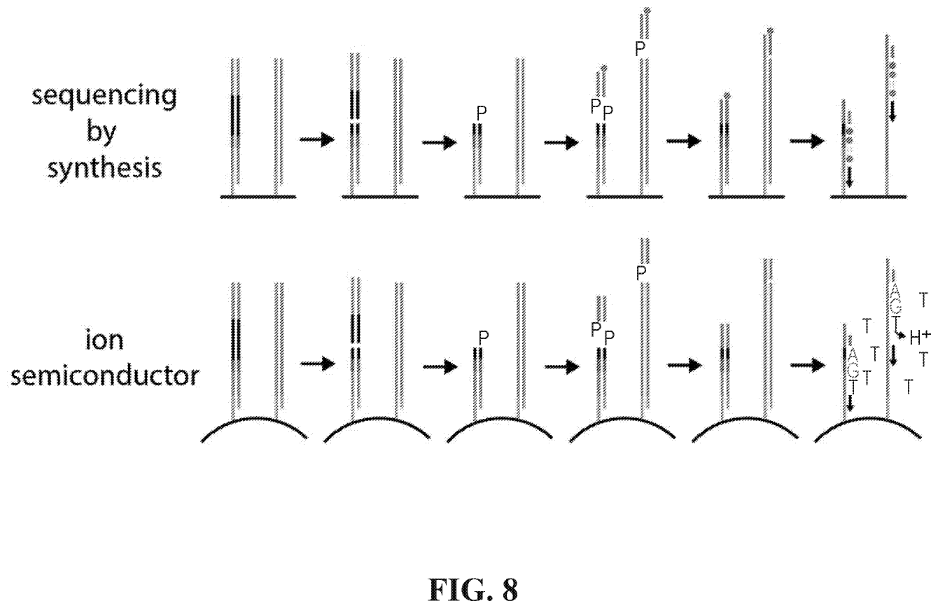

In embodiments where the nucleic acid fragments are labeled with a first and second adapter—each adapter comprising a different primer binding site—a pair of primers corresponding to the primer binding site in the first and second adapter is used to amplify the nucleic acid fragments. As in the previous embodiment, both nucleic acid fragments comprising a modification target site/sequence and nucleic acid fragments that do not comprise a modification target site/sequence can be amplified. The amplified nucleic acid sequences are then contacted with the modification agent. In certain example embodiments, the modification agent results in DSB at the modification sequence/site. In certain example embodiments, all nucleic acid fragment sequences—modified and unmodified—are sequenced. FIG. 6 provides a schematic example of such method. To distinguish between modified and unmodified nucleic acid fragments two additional ligations are carried out. Modified nucleic acid fragments will comprise two sub-populations. A first sub-population with the first adapter and a free unlabeled end. A first ligation adds a concatentation of a third adapter and the original second adapter to the free end. A second sub-population of nucleic acid fragments will comprise the second adapter and a free unlabeled end. The second ligation adds a concatentation of the third adapter and the original first adapter to the first end. Some modified nucleic acid fragments may end up with a second adapter at both ends or a first adapter at both ends. Modified nucleic acids with a desired orientation of a first adapter and a second adapter on opposing ends may be enriched through amplification, along with unmodified nucleic acids, using primers to the first and second adapter. The presence of the third adapter distinguishes modified nucleic acid fragments from unmodified nucleic acid fragments. Sequencing of the modified and unmodified sequences is then carried out to analyze DSBs and optionally to determine if any off target modification sites were generated by the modification agent. The modified sequences may be sequenced using a primer to the third adapter. The modified and unmodified sequences may be sequenced both using a primer to the first or second adapter. In other embodiments, only the modified nucleic acid fragments may be enriched. FIG. 5 provides a schematic example of such method. This may be achieved by only conducting a single ligation that adds the third adapter to each free end of the modified nucleic acid fragments and enriching for only those fragments comprising a third adapter. In certain example embodiments the third adapter may comprise a first adapter overhand and a second adapter overhang. This can be done to preserve the ability to sequence using certain sequencing technologies. For example, if the first adapter is a P7 adapter and the second adapter is a P5 adapter, ligation of a third adapter comprising first and second adapter overhangs preserves the ability to sequence the enriched modified fragments using SBS.

In a further exemplary embodiment a method of the invention for enrichment of nucleic acid molecules in which a nucleic acid break is performed as follows:

1. Prepare gDNA library with average size fragment of ˜400-500 bp (column extraction+sonication)

2. Ligate ‘ligation blocked adapters’ onto gDNA library fragments
Dideoxy terminators on adapters prevent further ligation of background gDNA in downstream steps
Adapters contain RA3 sequence (3′ Illumina adapter)
These can also be UMI tagged to label individual fragments of DNA in reaction

3. Purify

4. Incubate gDNA with CRISPR-Cas protein and candidate sgRNA to saturation
Cas9 cutting will expose ligation competent ends

5. Wash, Blunt, A-tail

6. Ligate adapters to cut DNA
adapters contain T7 primer as well as the RA5 (5′ Illumina adapter)

7. Purify and SPRI to eliminate excess T7 adapters

8. In vitro transcribe RNAs selectively enriched from break sites

9. DNase digest to get rid of all background DNA
Background DNA wouldn't cluster without both 5′ and 3′ Illumina sequences, but DNase digest provides further certainty

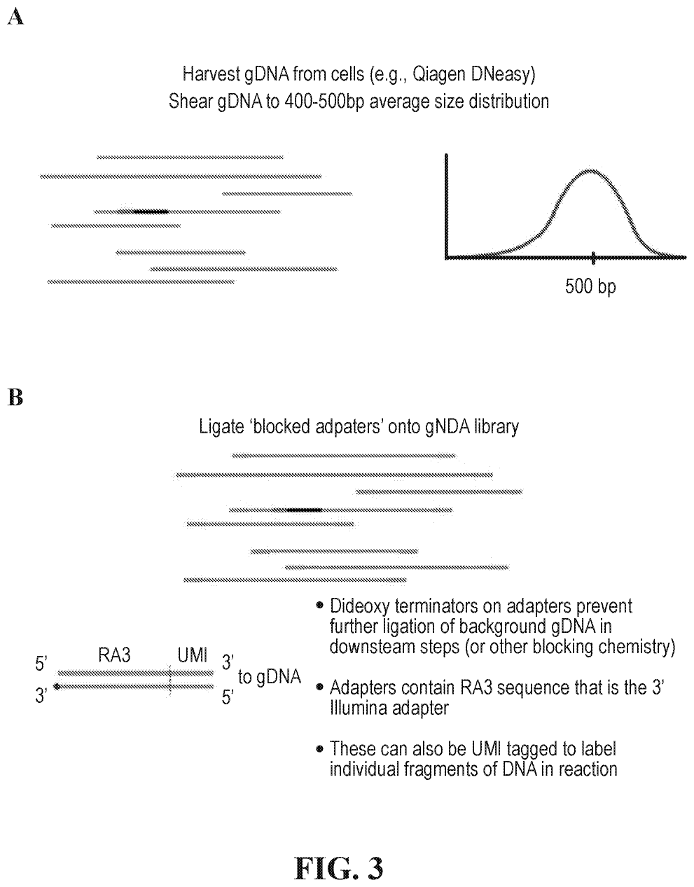

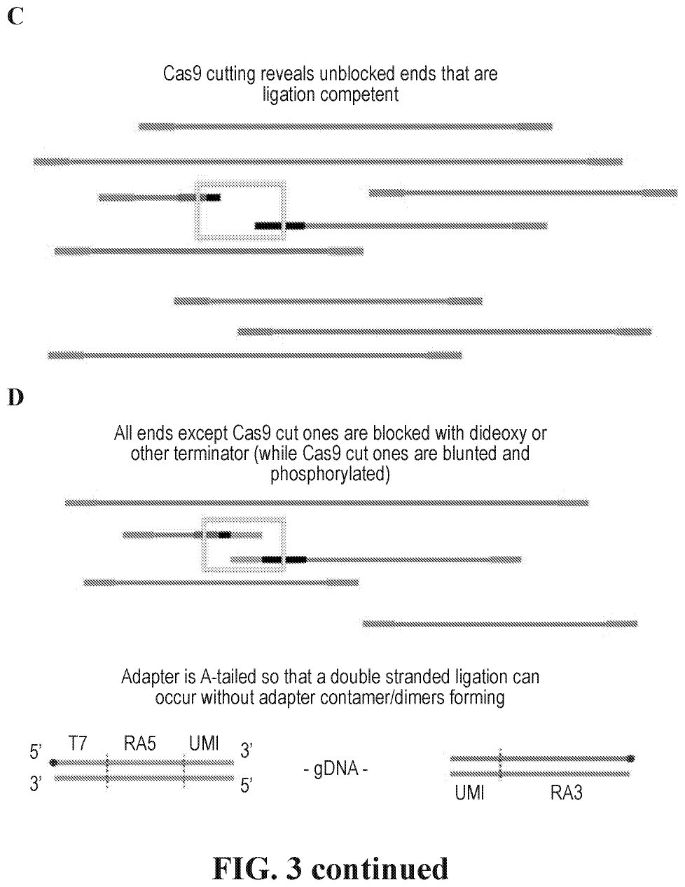

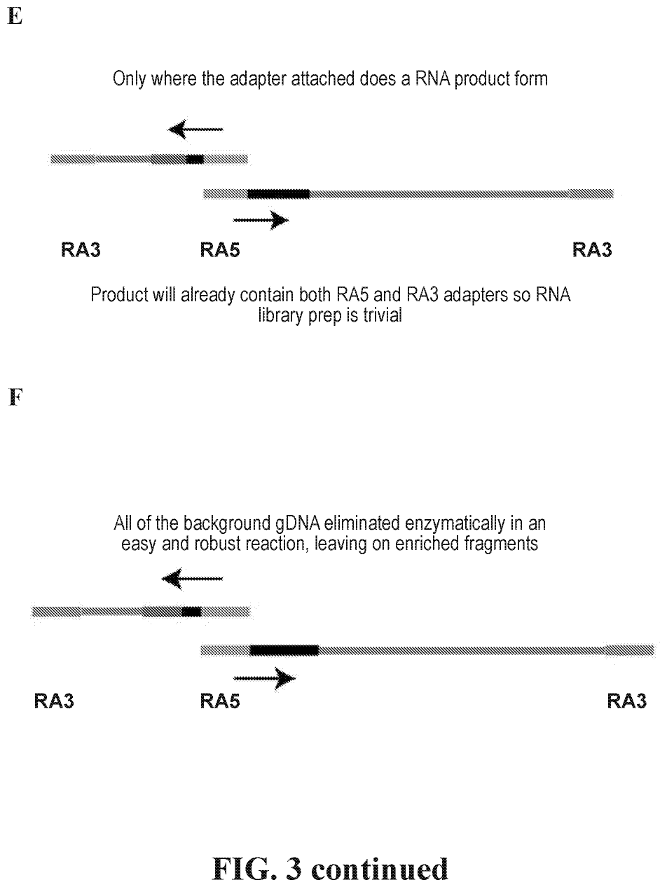

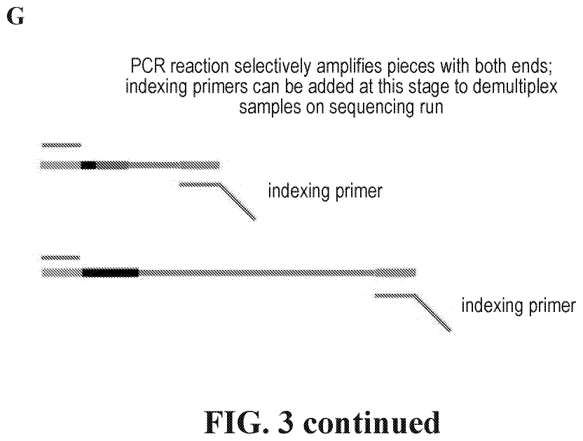

10. Prep sequencing library (RT and PCR)
By design these IVT′d RNA will contain the 5′ and 3′ sequences and are ready for direct RT and PCR to rapidly form sequencing library
Only the Cas9 induced break sites will contain fragments that have both 5′ and 3′ adapters that allow amplification by e.g. PCR.
   FIG. 3 provides a schematic overview of such method.

In particular embodiments, the methods for enrichment of modified nucleic acid molecules of the invention thus comprise amplifying nucleic acid molecules that have not been modified using said primers that bind to said first and second primer binding site.

Attachment of the adapter comprising the third primer binding site is preferably by ligation.

In particular embodiments, the steps of the enrichment methods disclosed herein are performed in the indicated order.

At least the first and second adapter used in the enrichment methods of the invention, which may be identical or different, comprises a ligation-blocking moiety. A “ligation-blocking moiety” refer to a moiety that prevents ligation of nucleotides to the polynucleotide comprising the moiety. Typically, such moieties also prevent attachment of nucleotides during e.g. amplification of a nucleotide sequence. Several ligation-blocking moieties are known in the art that can be present in the adapter used in the present invention. For example, an adapter may be modified at the 3′-terminal nucleotide by the addition of a 3′ deoxyribonucleotide residue, such as cordycepin, or a 2′,3′-dideoxyribonucleotide residue. Further examples include non-nucleotide linkages, alkane-diol modifications, a 2′3′-cyclic phosphate, and 3′ hydroxyl substitutions in the nucleotide, such as 3 ‘-phosphate, 3’-triphosphate or 3′-phosphate diesters with alcohols such as 3-hydroxypropyl. A preferred, but non-limiting, example of a ligation-blocking moiety is a dideoxynucleotide. Dideoxynucleotides (also referred to as 2′,3′ dideoxynucleotides) are chain-elongating inhibitors of DNA polymerase and block ligation of further polynucleotides. They are abbreviated as ddNTPs (ddGTP, ddATP, ddTTP and ddCTP). The absence of the 3′-hydroxyl group means that, no further nucleotides can be added as no phosphodiester bond can be created based on the fact that deoxyribonucleoside triphosphates allow DNA chain synthesis or ligation to occur through a condensation reaction between the 5′ phosphate (following the cleavage of pyrophospate) of the current nucleotide with the 3′ hydroxyl group of the previous nucleotide.

In the enrichment methods of the invention, adaptors comprising a ligation-blocking moiety are attached to both ends of the nucleic acid molecules prior to modification. The presence of these moieties on both ends of the nucleic acids ensures that further ligation of polynucleotides, such as adapters and modified nucleic acid molecules, to the nucleic acid molecules is not possible, e.g. during subsequent steps. Inducing a strand break, such as a SSB or a DSB, in the ligation-blocked nucleic acid molecules reveals unblocked ends that are ligation competent on one side of the modified nucleic acid molecules. The other ends and unmodified nucleic acid molecules remain ligation-blocked. As a result, the adapter comprising a third primer binding site is selectively attached only to modified, ligation-competent, nucleic acid molecules. Hence, the third primer binding site will only be present in modified nucleic acids and can be used to selectively amplify and/or sequence modified nucleic acids.

In certain example embodiments, the ligation-blocking moiety may further render the nucleic acid molecules labeled with the adapters on both the 5′ and 3′ ends of the nucleic acid molecules

In particular embodiments of the methods of the invention or enrichment of modified nucleic acid molecules, the nucleic acid molecules are RNA molecules, such as mRNA. In particular embodiments, wherein the plurality of nucleic acid molecules is a plurality of RNA molecules, said amplifying comprises reverse transcription using a primer that binds to the third primer binding site. In other embodiments, the nucleic acid molecules are DNA molecules, such as cDNA or genomic DNA. In a particular, preferred embodiment, the nucleic acid molecules comprise genomic DNA (gDNA). Preferably, said nucleic acid molecules comprising gDNA comprise gDNA fragments. In particular embodiments said gDNA is obtained from a patient in need of genome editing. In particular embodiments, wherein the plurality of nucleic acid molecules is a plurality of DNA molecules. Modified DNA molecules are transcribed into RNA using an RNA polymerase, where after DNA molecules are digested to enable selective amplification and sequencing of modified nucleic acid. Hence, in particular embodiments the adapter comprising a third primer binding site further comprises a DNA-dependent RNA polymerase promotor and said method further comprises, prior to said amplifying performing transcription of said one or more cleaved DNA molecules using said DNA-dependent RNA polymerase, resulting in one or more transcribed RNA molecules; and digesting DNA molecules, and wherein said amplifying comprises amplifying said one or more transcribed RNA molecules using primers that bind to said first or second primer binding site and to said third primer binding site. In particular embodiments, said amplifying comprises reverse transcription of said RNA molecules. Said digesting is advantageously performed using a DNase.

As describe herein above, the methods for enrichment of modified nucleic acid molecules, in particular wherein a strand break is induced, are advantageously used for enrichment prior to detection of modified nucleic acids. They are further particularly suitable for enrichments of nucleic acids wherein a strand break in induced for subsequent detection of off target activity of a targeted nuclease, for subsequent determination of cleavage efficiency of a targeted nuclease and for subsequent selection of a suitable guide RNA. Said method are advantageously performed in solution using enriched modified nucleic acid molecules prepared in accordance with the invention.

In one aspect the invention therefore provides a method for detecting a nucleic acid modification, comprising enriching one or more nucleic acid molecules wherein a nucleic acid modification is induced with a method according to the invention; and sequencing at least part of said amplified modified nucleic acid molecules. Advantageously said method is performed in solution.

In one aspect the invention provides a method for detecting a nucleic acid modification, comprising enriching one or more nucleic acid molecules wherein a nucleic acid modification is induced with a method according to the invention; sequencing at least part of said amplified modified nucleic acid molecules; and sequencing at least part of said amplified nucleic acid molecules that have not been modified. Advantageously said method is performed in solution.

In one aspect the invention provides a method for detecting off-target activity of a targeted nuclease specific for a selected target sequence, comprising enriching one or more nucleic acid molecules wherein a nucleic acid break is induced with a method according to the invention, wherein said agent comprises a targeted nuclease complex and detecting the presence of breaks in a sequence of said one or more nucleic acid molecules other than in said selected target sequence. Advantageously said method is performed in solution.

In one aspect the invention provides a method for determining cleavage efficiency of a targeted nuclease specific for a selected target sequence, comprising enriching one or more nucleic acid molecules wherein a nucleic acid break is induced with a method according to the invention, wherein said agent comprises a targeted nuclease complex; and determining a proportion of said plurality of nucleic acid molecules comprising a nucleic acid break at said selected target sequence. Advantageously said method is performed in solution.

In one aspect the invention provides a method for selecting a guide RNA from a plurality of guide RNAs specific for a selected target sequence, the method comprising enriching one or more nucleic acid molecules wherein one or more nucleic acid breaks are made with a method according to any one of claims 95-107 and 110-125, whereby said plurality of nucleic acid molecules is contacted with a plurality of RNA-guided nuclease complexes capable of inducing a nucleic acid break; and selecting a guide RNA based on location and/or amount of said nucleic acid breaks. Advantageously said method is performed in solution. In particular embodiments selecting comprises determining one or more locations in said one or more nucleic acid molecules comprising a break other than a location comprising said selected target sequence and selecting a guide RNA based on said one or more locations. In particular embodiments, selecting comprises determining a number of sites in said one or more immobilized nucleic acid molecules comprising a break other than a site comprising said selected target sequence and selecting a guide RNA based on said number of sites. A location in said one or more immobilized nucleic acid molecules comprising a break other than a location comprising said selected target sequence is herein also referred to as a location of an off-target break. Alternatively, said selecting comprises both determining the location of off-targets breaks and the number of locations of off-target breaks.

A modification that is induced in the methods of the invention for enrichment of modified nucleic acid molecules is preferably selected from the group consisting of an insertion, a replacement, a strand break and a recombination. In particular embodiments, the agent capable of inducing the modification is a chemical agent. Examples of such chemical agents include, but are not limited to, etoposide and teniposide. In particular embodiments, the agent capable of inducing the modification is an enzyme. Non-limiting examples of such enzymes are a nuclease, a (viral) integrase, a recombinases, a transposase, an argonaute. In a preferred embodiment, said enzyme comprises a nuclease.

In a particularly preferred embodiment, The application discloses systems for direct and unbiased detection of nucleic acid modifications induced by an agent in a nucleic acid molecule fixed to a solid surface. In particular, a system is disclosed in which the on target and off target cutting of a nuclease can be assessed in a direct and unbiased way using in vitro cutting of immobilized nucleic acid molecules. This way, the superset of all cleavage targets of an targeted nuclease can be captured in an unbiased way. In addition, the invention discloses methods and systems for a genome-wide, unbiased in vitro assay that allows selective amplification of the cut fragments, thus allowing much greater sensitivity per given read over comparable in vitro methods. With these methods it is possible to enrich for and detect targeted nuclease-induced breaks, without the need for whole genome sequencing (WGS) as is required in known methods such as Digenome-seq to get the requisite coverage for detecting the nuclease-induced cleavage sites. Despite the steady reduction in the cost of WGS, this is still an expensive proposition that can result in the loss of sensitivity due the limited read depth and background reads from the uncut genome. Furthermore, biases can be introduced in both the sequencing library preparation and readout of WGS that are avoided here.

In one aspect the invention provides a method for detecting a nucleic acid modification. The method can comprise: i) contacting one or more nucleic acid molecules immobilized on a solid support (immobilized nucleic acid molecules) with an agent capable of inducing a nucleic acid modification; and ii) sequencing at least part of said one or more immobilized nucleic acid molecules that comprises the nucleic acid modification using a primer specifically binding to a primer binding site. Advantageously the method comprises attaching an adapter comprising the primer binding site to the one or more immobilized nucleic acid molecules following contacting step i) and prior to sequencing step ii); alternatively or additionally, the one or more immobilized nucleic acid molecules that are contacted with the agent comprise an adapter comprising the primer binding site, e.g.: steps i) and ii) are performed wherein the one or more immobilized nucleic acid molecules that are contacted with the agent comprise an adapter comprising the primer binding site; or steps i) and ii) are performed with attaching an adapter comprising the primer binding site to the one or more immobilized nucleic acid molecules following contacting step i) and prior to sequencing step ii); or steps i) and ii) are performed with attaching an adapter comprising the primer binding site to the one or more immobilized nucleic acid molecules following contacting step i) and prior to sequencing step ii) and wherein the one or more immobilized nucleic acid molecules that are contacted with the agent comprise an adapter comprising the primer binding site.

Such methods allow for an unbiased, fast and comprehensive platform for analysis of modifications, both on-target and off-target, induced in cell-free DNA or RNA. The modifications are induced directly on nucleic acid fragments immobilized on a solid or semisolid surface, such as a sequencing platform, so that the sites of modification can be easily identified due to the nucleic acid molecules already being sequenced and registered. Because the modification is induced in the nucleic acid following library preparation, a superset of all targets is captured. The methods allow for analysis of genome-wide effects of induced modifications, in particular of genome editing applications such as targeted genome-editing nucleases. The methods are useful for a wide variety of applications, including analysis of off-target activity and efficiency of agents capable of inducing a modification, such as targeted nuclease complexes, and for selecting suitable guide RNAs specific for a selected target sequence for such targeted nuclease complexes. Such analyses are of particular high importance for therapeutic strategies involving genome-editing. For instance, the method of the invention can identify high-efficiency targeted nucleases that that manipulate a key therapeutic locus for initial therapeutic development. Further, prior to receiving therapy, the methods of the invention can be performed on a patient's own genomic DNA to analyze multiple candidate targets and to identify the target with the lowest risk for therapeutic intervention.

In a further aspect, the invention provides a method for detecting off-target activity of a targeted nuclease specific for a selected target sequence, the method comprising:
v. contacting a plurality of nucleic acid molecules immobilized on a solid support (immobilized nucleic acid molecules) with a complex comprising said targeted nuclease, thereby inducing one or more nucleic acid breaks;
vi. attaching an adapter comprising a primer binding site to one or more immobilized nucleic acid molecules comprising a nucleic acid break;
vii. sequencing at least part of said one or more immobilized nucleic acid molecules comprising a nucleic acid break using a primer specifically binding to said primer binding site;
viii. detecting the presence of breaks in a sequence of said one or more immobilized nucleic acid molecules other than in said selected target sequence.

In a further aspect the invention provides a method for determining cleavage efficiency of a targeted nuclease specific for a selected target sequence, the method comprising:
iv. contacting a plurality of nucleic acid molecules immobilized on a solid support (immobilized nucleic acid molecules) with a complex comprising said targeted nuclease, thereby inducing one or more nucleic acid breaks;
v. attaching an adapter comprising a primer binding site to one or more immobilized nucleic acid molecules comprising a nucleic acid break;
vi. determining cleavage efficiency of said plurality of immobilized nucleic acid molecules comprising a nucleic acid break at said selected target sequence.
   In particular embodiments, said determining is performed by determining a proportion of said plurality of immobilized nucleic acid molecules comprising a nucleic acid break at said selected target sequence. In particular embodiments, said determining is performed by sequencing at least part of said one or more immobilized nucleic acid molecules comprising a nucleic acid break using a primer specifically binding to said primer binding site. In particular embodiments, said determining is performed by determining a fluorescence intensity of said one or more immobilized nucleic acid molecules comprising said adapter which further comprises a fluorescent moiety. In one embodiment, said fluorescence intensity is determined cyclically, wherein each cycle comprises addition of said complex to said plurality of nucleic acid molecules followed by the step of determining fluorescence intensity.

In a further aspect, the invention provides a method for selecting a guide RNA from a plurality of guide RNAs specific for a selected target sequence, the method comprising:
v. contacting a plurality of nucleic acid molecules immobilized on a solid support (immobilized nucleic acid molecules) with a plurality of RNA-guided nuclease complexes capable of inducing a nucleic acid break, said plurality of RNA-guided nuclease complexes comprising a plurality of different guide RNA's, thereby inducing one or more nucleic acid breaks;
vi. attaching an adapter comprising a primer binding site to said one or more immobilized nucleic acid molecules comprising a nucleic acid break;
vii. sequencing at least part of said one or more immobilized nucleic acid molecules comprising a nucleic acid break using a primer specifically binding to said primer binding site.
viii. selecting a guide RNA based on location and/or amount of said one or more breaks.

In particular embodiments, step iv comprises determining one or more locations in said one or more immobilized nucleic acid molecules comprising a break other than a location comprising said selected target sequence (off-target breaks) and selecting a guide RNA based on said one or more locations. In particular embodiments, step v comprises determining a number of sites in said one or more immobilized nucleic acid molecules comprising off-target breaks and selecting a guide RNA based on said number of sites. In a further embodiment, step iv comprises both determining the location of off-targets breaks and the number of locations of off-target breaks.

In a preferred embodiment, the steps of the methods disclosed herein are performed in the indicated order.

In particular embodiments, the nucleic acid molecules are RNA molecules, such as mRNA. In other embodiments, the nucleic acid molecules are DNA molecules, such as cDNA or genomic DNA. In a particular, preferred embodiment, the nucleic acid molecules comprise genomic DNA (gDNA). In certain embodiments, the gDNA is fragmented into a plurality of smaller gDNA fragments. In particular embodiments said gDNA is obtained from a patient in need of genome editing.

In particular embodiments, the nucleic acid modification is selected from methylation, a mutation, a deletion, an insertion, a replacement, a ligation, an inversion, a digestion, a strand break and a recombination.

In particular embodiments, the agent capable of inducing a nucleic acid modification is a chemical agent. Examples of such chemical agents include, but are not limited to, etoposide and teniposide.

In particular embodiments, the agent capable of inducing a nucleic acid modification is a protein. Non-limiting examples of such proteins are a nuclease, a (viral) integrase, a recombinases, a transposase, an argonaute, a cytidine deaminase, a retron and a group II intron. In a preferred embodiment, said protein comprises a nuclease. In a particularly preferred embodiment, said agent comprises a targeted nuclease complex. Preferably the nucleic acid modification is a strand break, more preferably a SSB, a DSB or a nick, most preferably a DSB, and the agent comprises a nuclease, more preferably a targeted nuclease. In particular embodiments, said targeted nuclease complex comprises a zinc finger nuclease (ZFN), a transcription activator-like effector nuclease (TALEN) or CRISPR-Cas. In one embodiment said targeted nuclease complex comprises a RNA-directed nuclease complex. In one embodiment the targeted nuclease complex or the RNA-guided nuclease complex is a non-naturally occurring or engineered complex. In a preferred embodiment, said nuclease is selected from the group consisting of Cas9, Cpf1, C2c1, C2c2, C2c3, a group 29 nuclease, a group 30 nuclease and derivatives thereof. In a preferred embodiment, said targeted nuclease complex is a CRIPSR-Cas complex. In a particularly preferred embodiment, said CRIPSR-Cas complex comprises Cas9 or a modified Cas9.

In some embodiments, the methods comprise allowing a CRISPR complex to bind to the immobilized nucleic acid molecules to effect cleavage thereof, wherein the CRISPR complex comprises a nuclease complexed with a guide sequence hybridized or hybridizable to a target sequence within said immobilized nucleic acid molecules, wherein said guide sequence is linked to a tracr mate sequence which in turn hybridizes to a tracr sequence.

The methods provided herein allow for the simultaneous assessment of a plurality of candidate target sites as possible cleavage targets for any given nuclease, i.e. the methods of the invention are suitable for multiplexed analysis of multiple candidate target sites. Hence, in particular embodiments, the one or more immobilized nucleic acid molecules are contact with a plurality of targeted nuclease complexes, preferably with a plurality of different targeted nuclease complexes. Said plurality of targeted nuclease complexes may for instance comprises different guide RNAs specific for a single selected target sequence. Alternatively, or additionally, said plurality of targeted nuclease complexes may comprise different guide RNAs specific for different selected target sequences. Using a plurality of different targeted nuclease complexes allows for a comparison of the different complexes and selection of the most appropriate complex for the intended application. For instance, off target activity and cleavage efficiency of different targeted nuclease complexes can be assessed in a single assay. In certain example embodiments, the targeted nuclease complexes are CRISPR-Cas complexes.

In the methods of the invention one or more immobilized nucleic acid molecules are used. In a preferred embodiment, said one or more immobilized nucleic acid molecules comprise one or more clusters of immobilized nucleic acid molecules. In certain example embodiments, each cluster comprises multiple copies of a single immobilized nucleic acid molecule.

In one aspect, the invention provides a method for detecting a strand break, the method comprising:

vi. sequencing at least part of one or more clusters of nucleic acid molecules immobilized on a solid support (immobilized nucleic acid molecules);

vii. contacting said immobilized nucleic acid molecules with a targeted nuclease complex, resulting in immobilized nucleic acid molecules comprising a strand break;

viii. attaching an adapter comprising a primer binding site to said immobilized nucleic acid molecules comprising a strand break; and

ix. sequencing at least part of said one or more immobilized nucleic acid molecules using a primer specifically binding to said primer binding site, said part comprising said strand break;

x. comparing the sequences obtained prior to and subsequent to said contacting with a targeted nuclease complex.

In a preferred embodiment, the method further comprises sequencing at least part of said one or more immobilized nucleic acid molecules prior to said contacting with an agent capable of inducing a nucleic acid modification. In particular embodiments, the sequences obtained prior to and subsequent to contacting the immobilized nucleic acid molecules with an agent capable of inducing a nucleic acid modification are compared for each nucleic acid molecule or for each cluster of amplified immobilized nucleic acid molecules. Comparing said sequences allows for fast detection of the presence or absence of a nucleic acid modification in the specific nucleic acid molecule. Preferably, the methods of the invention are characterized in that no amplification is carried out between the two sequencing steps.

The methods of the invention comprise sequencing at least part of nucleic acid molecules, either prior to or following modification induced by an agent as defined herein, or both. When sequencing follows agent-induced modification, said part of the nucleic acid that is sequenced preferably comprises a nucleic acid sequence of said molecule that is sufficient to allow determining whether the nucleic acid molecule has been modified, i.e. comprises an insertion, deletion, mutation, strand break, inversion etc. Said part therefore preferably comprises the nucleic acid modification, meaning that said part comprises at least the site in the sequence of the nucleic acid molecule that has been modified. In case of sequencing prior to agent induced modification as described herein, said part preferably comprises the site in the sequence of the nucleic acid molecule where the modification will be induced or is likely to be induced. The parts that are sequenced further preferably comprise one or more, such as 5, 10 or 15 nucleotides flanking the site in the sequence that has been modified or where the modification will be induced or is likely to be induced. If the site in the sequence where the modification will be induced or is likely to be induced is unknown, essentially the entire nucleic acid molecule can be sequenced. In particular embodiments, the nucleic acid molecules are sequenced, either prior to or following modification induced by an agent as defined herein, or both.

In a particular embodiment, sequencing comprises sequencing by synthesis (SBS). An SBS method can generally comprise the following steps. 1. Break up DNA into manageable fragments of about 200 to about 600 base pairs. 2. Short sequences of DNA called adaptors, are attached to the DNA fragments. 3. The DNA fragments attached to adaptors are then made single stranded. This can be done by incubating the fragments with a base such as sodium hydroxide. 4. Once prepared, the DNA fragments are washed across a flowcell. The complementary DNA binds to primers on the surface of the flowcell and DNA that does not attach is washed away. 5. The DNA attached to the flowcell is then replicated to form small clusters of DNA with the same sequence. When sequenced, each cluster of DNA molecules will emit a signal that is strong enough to be detected by a camera. 6. Unlabeled nucleotide bases and DNA polymerase are then added to lengthen and join the strands of DNA attached to the flowcell. This creates ‘bridges’ of double-stranded DNA between the primers on the flow cell surface. 7. The double-stranded DNA is then broken down into single-stranded DNA using heat, leaving several million dense clusters of identical DNA sequences. 8. Primers and fluorescently-labelled terminators (terminators are a version of nucleotide base—A, C, G or T—that stop DNA synthesis) are added to the flow cell. 9. The primer attaches to the DNA being sequenced. 10. The DNA polymerase then binds to the primer and adds the first fluorescently-labelled terminator to the new DNA strand. Once a base has been added no more bases can be added to the strand of DNA until the terminator base is cut from the DNA. 11. Lasers are passed over the flowcell to activate the fluorescent label on the nucleotide base. This fluorescence is detected by a camera and recorded on a computer. Each of the terminator bases (A, C, G and T) give off a different color. 12. The fluorescently-labelled terminator group is then removed from the first base and the next fluorescently-labelled terminator base can be added alongside. And the process continues until a large number, e.g., millions of clusters have been sequenced. 13. The DNA sequence is analysed base-by-base during this sequencing, making it a highly accurate method. The sequence generated can then be aligned to a reference sequence, this looks for matches or changes in the sequenced DNA. SBS may include Next Generation Sequencing (NGS) and high throughput forms of SBS. In particular embodiments, sequencing comprises NGS, also referred to as high-throughput sequencing. Technologies for NGS are known in the art, examples include Illumina (Solexa) sequencing, Roche 454 sequencing, Ion torrent: Proton/PGM sequencing, and SOLiD sequencing.

In particular embodiments, the methods of the invention comprise amplification of said one or more immobilized nucleic acid molecules prior to said contacting with an agent capable of inducing a nucleic acid modification. Thereby, a plurality of immobilized nucleic acid molecules is produced. Such plurality of immobilized nucleic acid molecules resulting from amplification is herein also referred to as amplified immobilized nucleic acid molecules or a cluster of amplified immobilized nucleic acid molecules. Said amplification is preferably performed prior to contacting the immobilized nucleic acid molecules with an agent capable of inducing a nucleic acid modification so that potential bias resulting from such amplification is avoided.

Indeed, one of the main advantages of the methods of the invention is that essentially no manipulation (apart from converting single stranded nucleic acid molecules into double stranded nucleic acid molecules or vice versa) such as amplification is performed with the nucleic acid molecules after contacting with the agent capable of inducing a modification, which manipulations could introduce bias. Hence, in a preferred embodiment, the method does not comprise an amplification step after immobilized nucleic acid molecules have been contacted with an agent capable of inducing a nucleic acid modification such as a targeted nuclease complex and prior to sequencing said immobilized nucleic acid molecules thereafter.

In particular embodiments said amplifying comprises bridge amplification. In particular embodiments, the methods of the invention comprise:

h) allowing one or more nucleic acid molecules flanked by said first and second adapter to hybridize to one of said plurality of first or second immobilized oligonucleotides, whereby said first adapter comprises a sequence that is able to hybridize to said first immobilized oligonucleotides and said second adapter comprises a sequence that is complementary to a sequence that is able to hybridize to said immobilized second oligonucleotides;

and the bridge amplification comprises:

i) extending said first oligonucleotide with a polymerase whereby said one or more single stranded nucleic acid molecules flanked by a first and a second adapter are used as a template;

j) removing said one or more single stranded nucleic acid molecules flanked by a first and a second adapter used as a template resulting in one or more single stranded immobilized nucleic acid molecules;

k) hybridizing said one or more single stranded immobilized nucleic acid molecules to one of said plurality of immobilized second oligonucleotides;

1) extending said second oligonucleotide with a polymerase resulting in one or more double stranded immobilized nucleic acid molecules;

m) denaturing said one or more double stranded immobilized nucleic acid molecules to produce a plurality of immobilized single stranded nucleic acid molecules; and

n) repeating steps d-f at least once.

Steps d-f are preferably repeated multiple times so that a cluster of identical nucleic acid molecules is obtained for each of the plurality of nucleic acid molecules. Said cluster preferably comprises sufficient nucleic acid molecules to allow sequencing. Each cluster may contain for instance one million copies of the original nucleic acid molecule.

In particular embodiments, methods of the invention for detecting off target activity of a targeted nuclease specific for a selected target sequence, for determining cleavage efficiency of a targeted nuclease specific for a selected target sequence or for selecting a guide RNA from a plurality of guide RNAs specific for a selected target sequence further comprise:

h) allowing one or more nucleic acid molecules flanked by a first and a second adapter to hybridize to one of said plurality of first or second immobilized oligonucleotides, whereby said first adapter comprises a sequence that is able to hybridize to said first immobilized oligonucleotides and said second adapter comprises a sequence that is complementary to a sequence that is able to hybridize to said immobilized second oligonucleotides;

and the bridge amplification comprises:

i) extending said first oligonucleotide with a polymerase whereby said single stranded nucleic acid molecules flanked by a first and a second adapter are used as a template;

j) removing said single stranded nucleic acid molecules flanked by a first and a second adapter used as a template resulting in a plurality of single stranded immobilized nucleic acid molecules;

k) hybridizing said one or more single stranded immobilized nucleic acid molecules to one of said plurality of immobilized second oligonucleotides;

1) extending said second oligonucleotide with a polymerase resulting in a plurality of double stranded immobilized nucleic acid molecules;

m) denaturing said plurality of double stranded immobilized nucleic acid molecules to produce a plurality of immobilized single stranded nucleic acid molecules; and

n) repeating steps d-f at least once.

Steps d-f are preferably repeated multiple times so that a cluster of identical nucleic acid molecules is obtained for each of the plurality of nucleic acid molecules. Said cluster preferably comprises sufficient nucleic acid molecules to allow sequencing. Each cluster may contain for instance one million copies of the original nucleic acid molecule.

In particular embodiments, the nucleic acid molecules are attached to said solid support via a chemical or protein linker.

In particular embodiments, the solid support comprises clusters of immobilized nucleic acids. In some embodiments, nucleic acid molecules are amplified prior to immobilization on the solid support. For instance, amplification prior to immobilization comprises emulsion amplification, which is particularly suitable to obtain clusters of nucleic acid molecules comprising multiple copies of the same original nucleic acid molecule. In particular embodiments, a solid support comprising a plurality of chemical or protein moieties is used in a method of the invention and the method comprises, prior to the contacting step i, allowing one or more nucleic acid molecules flanked by a first and a second adapter, wherein at least one of the adapters comprises a chemical or biological moiety capable of binding to said chemical or biological moieties of said solid support, to bind to said solid support. In particular embodiments the methods of the invention method comprises prior to said contacting step i: amplification of one or more nucleic acid molecules flanked by a first adapter comprising a first primer binding site and a second adapter comprising a second primer binding site in a droplet using primers specifically binding to said primer binding sites, wherein at least one of said primers comprises a chemical or biological moiety capable of binding to a solid support; and allowing said amplified nucleic acid molecules to bind to said solid support.

The methods of the invention use an adapter comprising a primer binding site. Said primer binding site is preferably used in amplification and/or sequencing of the immobilized nucleic acid molecules. For instance, the immobilized nucleic acid molecule (prior to modification) may be flanked on either end of the fragment by adapters comprising a different primer binding site. These primer binding sites can be used to amplify the immobilized nucleic acid molecules prior to contacting with the modification inducing agent, for instance by bridge amplification as described herein elsewhere. Further, if one of the adapters comprising a primer binding site is removed as a result of the nucleic acid modification induced by the agent, such as a strand break, another, third, adapter can be attached to the modified nucleic acid molecules, for instance at the site of the strand break. Such attachment is optionally executed after blunt ending of modified nucleic acid molecules. Such third adapter an be used for sequencing of the nucleic acid molecules. In particular embodiments of the invention only nucleic acid molecules wherein a modification such as a strand break is induced are sequenced. Thus, in preferred embodiment, nucleic acid molecules wherein a modification such as a strand break is induced are selectively sequenced. This is for instance achieved by using an adapter for attaching to modified, e.g. cleaved, nucleic acid molecules that is distinguishable from the adapters flanking the nucleic acid molecules prior to inducing the modification. In particular embodiments of the invention nucleic acid molecules are sequenced both prior to and following modification. A comparison of the sequences of the same nucleic acid molecules prior to and following inducing the modification can be made.

In particular embodiments, a method of the invention for detecting a nucleic acid modification comprises attaching an adapter comprising the primer binding site to said one or more immobilized nucleic acid molecules following step i and prior to sequencing in step ii. Such method is particularly suitable if the nucleic acid modification comprises a DSB, a SSB or a nick, which results in cleavage of the immobilized nucleic acid molecules. In that case an adapter comprising the primer binding site already present on the immobilized nucleic acid molecules is potentially cleaved of, resulting in nucleic acid molecules lacking a primer binding site. Hence, in a method wherein said nucleic acid modification comprises a strand bread, the adapter comprising the primer binding site is preferably attached to the nucleic acid molecule after contacting with the agent capable of inducing the strand break.

In particular embodiments, in such methods the adapter comprising the primer binding site is specific for the immobilized nucleic acid molecules that have been cleaved. That way the adapter is only attached to the immobilized nucleic acid molecules wherein a strand break is induced and not to unmodified nucleic acid molecules. This is for instance achieved by phosphatase treatment of the immobilized nucleic acid molecules prior to contacting with the agent capable of inducing a nucleic acid modification, in particular a strand break. Hence, in particular embodiments, one or more immobilized nucleic acid molecules are unphosphorylated. Further, the one or more immobilized nucleic acid molecules comprising a nucleic acid modification, preferably a strand break, are preferably phosphorylated prior to attaching to said adapter comprising a primer binding site.

In particular embodiments, wherein said nucleic acid modification comprises a DSB which results in an overhang, said DSB is blunt ended before attaching to said adapter comprising a primer binding site.

In particular embodiments, in a method of the invention for detecting a nucleic acid modification the one or more immobilized nucleic acid molecules that are contacted with said agent already comprise an adapter comprising said primer binding site. Such method is particularly suitable if the nucleic acid modification does not result in a strand break or cleavage of the nucleic acid molecules. Such nucleic acid modifications for instance comprise an insertion, a deletion, a substitution or a rearrangement. Alternatively, the adapter comprising the primer binding site is attached to the nucleic acid molecules after contacting with the agent capable of inducing such modification. In both alternatives, the primer binding site will be available for use in sequencing the immobilized nucleic acid molecules.

In particular embodiments, the adapter comprising the primer binding site comprises a modified nucleic acid, a chemical moiety, an affinity moiety, or a fluorescent moiety. Such modified nucleic acid or chemical, affinity or fluorescent moiety allows for detection of immobilized nucleic acid molecules other than by sequencing. In such methods, the nucleic acid modification preferably comprises a strand break and the adapter is attached to the immobilized nucleic acid molecules after contacting with the agent capable of inducing the strand break.

In particular embodiments, the solid support is selected from a chip, an array a flow cell, a microwell, a microwell comprising an affinity treated surface and a bead, such as an immobilized affinity bead. In a preferred embodiment, the solid support is a flow cell or a bead, more preferably a flow cell.

In particular embodiments, the immobilized nucleic acid molecules comprise a “unique molecular identifier” (UMI). The term “UMI” refers to a sequencer linker used in a method that uses molecular tags to detect and quantify unique amplified products. In the method of the present invention, a UMI may be used to distinguish nucleic acid molecules wherein a modification, such as a break, has been induced from nucleic acid molecules not containing said nucleic acid modification. Alternatively, they may be used to distinguish nucleic acid molecules wherein a modification, such as a break, has been induced in a selected target sequence from nucleic acid molecules wherein the modification has been induced in a sequence of said one or more immobilized nucleic acid molecules other than in said selected target sequence.

In particular embodiments, the one or more immobilized nucleic acid molecules comprise a barcode, preferably a DNA barcode if the nucleic acid is DNA or an RNA barcode if the nucleic acid is RNA. The term barcode as used herein, refers to any unique, non-naturally occurring, nucleic acid sequence that may be used to identify the originating genome of a nucleic acid fragment.

In one aspect, the invention provides a kit of parts for executing a method according to the invention.

In one aspect, the invention provides a kit of parts comprising a solid support comprising one or more nucleic acid molecules immobilized thereon and an agent capable of inducing a nucleic acid modification. Such kit of parts is particularly suitable for detecting nucleic acid modifications in nucleic acid molecules, such as genomic DNA, in accordance with the methods of the invention.

In particular embodiments, the nucleic acid modification is selected from methylation, a mutation, a deletion, an insertion, a replacement, a ligation, a digestion, a strand break and a recombination. In particular embodiments, the agent of said kit of parts is selected from a nuclease, a chemical agent, a (viral) integrase, a recombinases, a transposase, an argonaute, a cytidine deaminase, a retron and a group II intron. Preferably the nucleic acid modification is a strand break, more preferably a SSB, a DSB or a nick and the agent in said kit of parts comprises a nuclease, more preferably a targeted nuclease. In particular embodiments, the agent comprises a targeted nuclease complex. In particular embodiments, said complex comprises a ZFN, TALEN or CRISPR-Cas. In a preferred embodiment, said nuclease is selected from the group consisting of Cas9, Cpf1, C2c1, C2c2, C2c3, a group 29 nuclease, a group 30 nuclease and derivatives thereof. In a preferred embodiment, said targeted nuclease complex is a CRIPSR-Cas complex.

In certain example embodiments, a genomic DNA library is prepared and the gDNA fragments are provided with an adapter comprising a primer binding site on each end, referred as the first and second adapter. For instance, the first adapter is a P5 adapter and is attached 5′ of the fragments and the second adapter is a P7 adapter and is attached 3′ of the fragments. The adapter-flanked fragment are subsequently annealed to a flow cell comprising oligo's that are able to hybridize to the adapter sequences. Bridge amplification, described herein elsewhere, is performed to provide clusters of amplified nucleic acids for each gDNA fragment annealed to the flow cell. One or both of the strands of the resulting amplified double stranded DNA are subsequently sequenced, for instance using SBS, referred to as the R1 and R2 reads. Following sequencing, the remaining strand is converted into double stranded DNA. The amplified nucleic acid sequences are then contacted with the modification agent. In certain example embodiments, the modification agent results in a DSB in the nucleic acid fragments. The nucleic acid fragments with DSBs are then labeled attached at one end via an adapter to the flow cell and are unlabeled on a new terminal end generated at the site of the DSB. A third adapter comprising a primer binding site may then be added to the new terminal end of the modified nucleic acid fragments. In certain embodiments, the third adapter will also be added to the unmodified nucleic acid fragments, i.e. to either the first or second adapter that is not attached to the flow cell. In certain embodiments, the third adapter is selectively attached to modified nucleic acid fragments only and not to unmodified fragments. Sequencing of the modified and optionally unmodified sequences is then carried out to analyze DSBs and optionally to determine if any off target modification sites were generated by the modification agent. Either only the modified fragments or both the modified and unmodified fragments may be sequenced using a primer to the third adapter. In the unmodified fragments, the third adapter is attached directly to the first or second adapter which is readily detected when sequencing, allowing direct filtering out of the unmodified sequences. The modified nucleic acids can be analyzed to determine cleavage sites in the genome. FIG. 1 provides a schematic overview of such method.

In one aspect, the invention provides a kit of parts comprising a targeted nuclease and a solid support. Such kit of parts is particularly suitable for detecting nucleic acid modifications in nucleic acid molecules, such as genomic DNA, in accordance with the methods of the invention. Advantageously, said solid support is configured to allow attachment of nucleic acid molecules. Hence, in particular embodiments, the solid support comprises a plurality of first and second oligonucleotides immobilized thereto. In particular embodiments, the kit of parts further comprises a first adapter comprising a sequence that is able to hybridize to said first immobilized oligonucleotides and a second adapter comprising a sequence that is complementary to a sequence that is able to hybridize to said immobilized second oligonucleotides.

Alternatively, the kit of parts comprises a solid support comprising a plurality of chemical or protein linkers. Such linkers can be used to bind nucleic acid molecules functionalized with a chemical or protein moiety able to bind to such linkers. Hence, in particular embodiments, such kit of parts further comprises a first adapter comprising a first primer binding site and a second adapter comprising a second primer binding site, wherein at least one of said adapters comprises a chemical or biological moiety capable of binding to said chemical or protein linkers.

Such kit of parts comprising a solid support configured to allow attachment of nucleic acid molecules are particularly suitable for detecting nucleic acid modifications in nucleic acid obtained from a patient, such as a patient in need of genomic editing. Indeed, the genomic DNA of a patient can be fragmented, followed by attachment of the first and second adapter to the genomic DNA fragments on both ends of the fragments. Subsequently the genomic DNA fragments flanked by the first and second adapter can be immobilized to the solid support by attaching to the plurality of first or second oligonucleotides immobilized thereon. Method to immobilize nucleic acid fragments to the solid support are described herein below.

In particular embodiments, the kit of parts comprising a targeted nuclease and a solid support comprising a plurality of first and second oligonucleotides immobilized thereto further comprises one or more nucleic acid molecules. In particular embodiments, said nucleic acid molecule are flanked by a first and a second adapter, whereby said first adapter comprises a sequence that is able to hybridize to said first immobilized oligonucleotides and said second adapter comprises a sequence that is complementary to a sequence that is able to hybridize to said immobilized second oligonucleotides.

In particular embodiments, the nucleic acid molecules comprised in a kit of part according to the invention comprise RNA. In particular embodiments, the nucleic acid molecules comprised in a kit of part according to the invention comprise DNA. In particular embodiments, the nucleic acid molecules comprised in a kit of part according to the invention are double stranded. In particular embodiments, the nucleic acid molecules comprised in a kit of part according to the invention are single stranded. In a particular, preferred, embodiment, the nucleic acid molecules comprise genomic DNA (gDNA). Preferably, said nucleic acid molecules comprising gDNA comprise gDNA fragments.

A kit of part of the invention may further contain one or more of the components such as the primers and enzymes necessary for the reaction chemistry and sequencing performed in the assay's of the invention. Hence, in particular embodiments, a kit of parts according to the invention further comprises one or more components selected from the group consisting of one or more primers, a DNA or RNA polymerase, a restriction enzyme, a ligase, an exonuclease, a mixture of nucleotides and labelled nucleotides. Said labelled nucleotides are for instance adenine, guanine, cytosine, thymine and/or uracil, whereby each nucleotide is labelled with a different fluorescent moiety. Such fluorescently labelled nucleotides are particularly suitable for sequencing by synthesis as explained in more detail herein below.

The nucleotides encompasses within a kit of parts of the invention are suitably modified, for instance modulation of ligation and nucleotide manipulation purposes. Hence, in particular embodiments, the nucleotides or fluorescently labeled nucleotides are modified nucleotides. Non-limiting examples of modified nucleotides are dideoxy nucleotides or nucleotides comprising a phosphorothiate linkage. Dideoxynucleotides (also referred to as 2′,3′ dideoxynucleotides) are chain-elongating inhibitors of DNA polymerase and block ligation of further polynucleotides. They are abbreviated as ddNTPs (ddGTP, ddATP, ddTTP and ddCTP). The absence of the 3′-hydroxyl group means that, no further nucleotides can be added as no phosphodiester bond can be created based on the fact that deoxyribonucleoside triphosphates allow DNA chain synthesis or ligation to occur through a condensation reaction between the 5′ phosphate (following the cleavage of pyrophospate) of the current nucleotide with the 3′ hydroxyl group of the previous nucleotide. A phosphorothioate linkage substitutes a sulfur atom for a non-bridging oxygen in the phosphate backbone of an polynucleotide. This modification renders the internucleotide linkage resistant to nuclease degradation. Phosphorothioate linkages can typically be introduced between the last 3-5 nucleotides at the 5′- or 3′-end of the polynucleotide.

In particular embodiments, the solid support encompassed in a kit of parts of the invention is selected from a chip, an array a flow cell, a microwell, a microwell comprising an affinity treated surface and a bead, such as an immobilized affinity bead. In a preferred embodiment, the solid support is a flow cell or a bead, more preferably a flow cell or a bead.

In one aspect the invention provides method for enrichment of one or more nucleic acid molecules wherein a nucleic acid modification is made, the method comprising contacting a plurality of nucleic acid molecules with an agent capable of inducing a nucleic acid modification, wherein said nucleic acid molecules are flanked by a first adapter comprising a first primer binding site and a ligation-blocking moiety and a second adapter comprising a second primer binding site and a ligation-blocking moiety, resulting in one or more modified nucleic acid molecules; and amplifying said one or more cleaved nucleic acid molecules comprising said adapter using a primer that binds to said first or second primer binding site and a primer that binds to a third primer binding site. Advantageously the method comprises attaching an adapter comprising the third primer binding site to said one or more modified nucleic acid molecules following the contacting step and prior to amplifying in step ii); alternatively or additionally, the modification comprises insertion of an adapter comprising the third primer binding site, e.g.: steps i) and ii) are performed wherein the modification comprises insertion of an adapter comprising the third primer binding site; or steps i) and ii) are performed with attaching an adapter comprising the third primer binding site to the one or more modified nucleic acid molecules following said contacting step and prior to amplifying in step ii); or steps i) and ii) are performed with attaching an adapter comprising the third primer binding site to the one or more modified nucleic acid molecules following the contacting step and prior to amplifying in step ii)) and wherein the modification comprises insertion of an adapter comprising the third primer binding site. Advantageously said method is performed in solution.

Such method allows for specific enrichment of nucleic acid molecules that have been modified, for instance by inducing a strand break, in vitro. Such methods are highly sensitive. In addition, they can easily be multiplexed, i.e. the methods allo for enrichment of multiple modification is possible in a single assay. The methods for enrichment of nucleic acid molecules wherein a nucleic acid modification has been made are particularly suitable for use in methods and assays for determining off target activity or cleavage efficiency of targeted (endo)nucleases in genomic DNA in solution. Enrichment of modified, e.g. cleaved, nucleic acid molecules avoids the need to perform whole genome sequencing in order to monitor and analyze targeted nuclease activity. Such whole genome sequencing is necessary in the only in vitro off-target detection assay that is currently available, i.e. Digenome-seq. The enrichment methods of the invention, followed by detection of strand breaks are particularly suitable for use as a control in cell-based off target assay's such as BLISS/BLESS.

In particular embodiments, the first and second primer binding sites are identical. This is for instance achieved if the first and second adapter are identical. Such methods are particularly suitable for enrichment and specific sequencing of modified nucleic acids, without sequencing unmodified nucleic acids. In other embodiments, the first and second primer binding sites are different. This is for instance achieved if the first and second adapter are different. Such method provides at least two possibilities for further processing: a first one for enrichment and specific sequencing of modified nucleic acids, without sequencing unmodified nucleic acids, and a second one for enrichment and sequencing of both modified and unmodified nucleic acids. Therefore, in particular embodiments wherein said first and second primer binding sites are different, the adapter comprising a third primer binding site further comprises a fourth primer binding site, which may be identical to the first or second primer binding site. In other embodiments wherein said first and second primer binding sites are different, the primer that binds to the third primer binding site comprises a fifth primer binding site, which may be identical to the first or second primer binding site. Amplification with such primer creates an overhang with an additional primer binding site.

In certain example embodiments, the nucleic acids to be tested are fragmented using fragmentation methods known in the art and discussed elsewhere herein to yield a plurality of smaller nucleic acid fragments. One or more smaller nucleic acid fragments may comprise modification target sites/sequences. In certain example embodiments, the test nucleic acid to be fragmented is genomic DNA. Each resulting fragmented nucleic strand is then labeled on each terminal end with an adapter. The adapter comprises a primer binding site. In one example embodiment, each nucleic acid fragment is labeled with the same adapter on each terminal end. In another example embodiment, each nucleic acid fragment is labeled on a first terminal end with a first adapter and on the second terminal end with a second adapter. The fragmented nucleic acids are then amplified.

In embodiments where the nucleic acid fragments are labeled with the same first adapter on both terminal ends—each adapter comprising the same primer binding site—a single primer may be used to amplify the nucleic acid fragments. Thus, both nucleic acid fragments comprising a modification target site/sequence and nucleic acid fragments that do not comprise a modification target site/sequence can be amplified. The amplified nucleic acid sequences are then contacted with the modification agent. In certain embodiments, the modification agent results in a double-stranded break in nucleic acid fragments. Those nucleic acid fragments with DSBs are then labeled one end with the original adapter and unlabeled on a new terminal end generated at the site of the DSB. A second adapter comprising a primer binding site may then be added to the new terminal end of the modified nucleic acid fragments. The modified nucleic acid fragments may then be enriched by amplification using a pair of primers corresponding to the primer binding sites in the first and second adapters. In certain example embodiments, a further, third, adapter may then be ligated to the second adapter for purposes of sequencing. For example, if the first adapter is a P7 adapter, the third adapter may be a P5 adapter to enable sequencing by synthesis (SBS) of the modified nucleic acid fragments. Sequencing of the modified nucleic acids is for instance carried out to analyze DSBs and optionally to determine if any off target modification sites were generated by the modification agent. FIG. 4 provides a schematic overview of an example of en enrichment method as described herein wherein first and second primer binding sites that are part of adapter flanking the nucleic acid molecule are identical.

In embodiments where the nucleic acid fragments are labeled with a first and second adapter—each adapter comprising a different primer binding site—a pair of primers corresponding to the primer binding site in the first and second adapter is used to amplify the nucleic acid fragments. As in the previous embodiment, both nucleic acid fragments comprising a modification target site/sequence and nucleic acid fragments that do not comprise a modification target site/sequence can be amplified. The amplified nucleic acid sequences are then contacted with the modification agent. In certain example embodiments, the modification agent results in DSB at the modification sequence/site. In certain example embodiments, all nucleic acid fragment sequences—modified and unmodified—are sequenced. FIG. 6 provides a schematic example of such method. To distinguish between modified and unmodified nucleic acid fragments two additional ligations are carried out. Modified nucleic acid fragments will comprise two sub-populations. A first sub-population with the first adapter and a free unlabeled end. A first ligation adds a concatentation of a third adapter and the original second adapter to the free end. A second sub-population of nucleic acid fragments will comprise the second adapter and a free unlabeled end. The second ligation adds a concatentation of the third adapter and the original first adapter to the first end. Some modified nucleic acid fragments may end up with a second adapter at both ends or a first adapter at both ends. Modified nucleic acids with a desired orientation of a first adapter and a second adapter on opposing ends may be enriched through amplification, along with unmodified nucleic acids, using primers to the first and second adapter. The presence of the third adapter distinguishes modified nucleic acid fragments from unmodified nucleic acid fragments. Sequencing of the modified and unmodified sequences is then carried out to analyze DSBs and optionally to determine if any off target modification sites were generated by the modification agent. The modified sequences may be sequenced using a primer to the third adapter. The modified and unmodified sequences may be sequenced both using a primer to the first or second adapter. In other embodiments, only the modified nucleic acid fragments may be enriched. FIG. 5 provides a schematic example of such method. This may be achieved by only conducting a single ligation that adds the third adapter to each free end of the modified nucleic acid fragments and enriching for only those fragments comprising a third adapter. In certain example embodiments the third adapter may comprise a first adapter overhand and a second adapter overhang. This can be done to preserve the ability to sequence using certain sequencing technologies. For example, if the first adapter is a P7 adapter and the second adapter is a P5 adapter, ligation of a third adapter comprising first and second adapter overhangs preserves the ability to sequence the enriched modified fragments using SBS.

In a further exemplary embodiment a method of the invention for enrichment of nucleic acid molecules in which a nucleic acid break is performed as follows:

1. Prepare gDNA library with average size fragment of ˜400-500 bp (column extraction+sonication)

2. Ligate ‘ligation blocked adapters’ onto gDNA library fragments
Dideoxy terminators on adapters prevent further ligation of background gDNA in downstream steps
Adapters contain RA3 sequence (3′ Illumina adapter)
These can also be UMI tagged to label individual fragments of DNA in reaction

3. Purify

4. Incubate gDNA with CRISPR-Cas protein and candidate sgRNA to saturation
Cas9 cutting will expose ligation competent ends

5. Wash, Blunt, A-tail

6. Ligate adapters to cut DNA
adapters contain T7 primer as well as the RA5 (5′ Illumina adapter)

7. Purify and SPRI to eliminate excess T7 adapters

8. In vitro transcribe RNAs selectively enriched from break sites

9. DNase digest to get rid of all background DNA
Background DNA wouldn't cluster without both 5′ and 3′ Illumina sequences, but DNase digest provides further certainty

10. Prep sequencing library (RT and PCR)
By design these IVT′d RNA will contain the 5′ and 3′ sequences and are ready for direct RT and PCR to rapidly form sequencing library
Only the Cas9 induced break sites will contain fragments that have both 5′ and 3′ adapters that allow amplification by e.g. PCR.
   FIG. 3 provides a schematic overview of such method.

In particular embodiments, the methods for enrichment of modified nucleic acid molecules of the invention thus comprise amplifying nucleic acid molecules that have not been modified using said primers that bind to said first and second primer binding site.

Attachment of the adapter comprising the third primer binding site is preferably by ligation.

In particular embodiments, the steps of the enrichment methods disclosed herein are performed in the indicated order.

At least the first and second adapter used in the enrichment methods of the invention, which may be identical or different, comprises a ligation-blocking moiety. A “ligation-blocking moiety” refer to a moiety that prevents ligation of nucleotides to the polynucleotide comprising the moiety. Typically, such moieties also prevent attachment of nucleotides during e.g. amplification of a nucleotide sequence. Several ligation-blocking moieties are known in the art that can be present in the adapter used in the present invention. For example, an adapter may be modified at the 3 ‘-terminal nucleotide by the addition of a 3’ deoxyribonucleotide residue, such as cordycepin, or a 2′,3′-dideoxyribonucleotide residue. Further examples include non-nucleotide linkages, alkane-diol modifications, a 2′3′-cyclic phosphate, and 3′ hydroxyl substitutions in the nucleotide, such as 3 ‘-phosphate, 3’-triphosphate or 3′-phosphate diesters with alcohols such as 3-hydroxypropyl. A preferred, but non-limiting, example of a ligation-blocking moiety is a dideoxynucleotide. Dideoxynucleotides (also referred to as 2′,3′ dideoxynucleotides) are chain-elongating inhibitors of DNA polymerase and block ligation of further polynucleotides. They are abbreviated as ddNTPs (ddGTP, ddATP, ddTTP and ddCTP). The absence of the 3′-hydroxyl group means that, no further nucleotides can be added as no phosphodiester bond can be created based on the fact that deoxyribonucleoside triphosphates allow DNA chain synthesis or ligation to occur through a condensation reaction between the 5′ phosphate (following the cleavage of pyrophospate) of the current nucleotide with the 3′ hydroxyl group of the previous nucleotide.

In the enrichment methods of the invention, adaptors comprising a ligation-blocking moiety are attached to both ends of the nucleic acid molecules prior to modification. The presence of these moieties on both ends of the nucleic acids ensures that further ligation of polynucleotides, such as adapters and modified nucleic acid molecules, to the nucleic acid molecules is not possible, e.g. during subsequent steps. Inducing a strand break, such as a SSB or a DSB, in the ligation-blocked nucleic acid molecules reveals unblocked ends that are ligation competent on one side of the modified nucleic acid molecules. The other ends and unmodified nucleic acid molecules remain ligation-blocked. As a result, the adapter comprising a third primer binding site is selectively attached only to modified, ligation-competent, nucleic acid molecules. Hence, the third primer binding site will only be present in modified nucleic acids and can be used to selectively amplify and/or sequence modified nucleic acids.

In certain example embodiments, the ligation-blocking moiety may further render the nucleic acid molecules labeled with the adapters on both the 5′ and 3′ ends of the nucleic acid molecules

In particular embodiments of the methods of the invention or enrichment of modified nucleic acid molecules, the nucleic acid molecules are RNA molecules, such as mRNA. In particular embodiments, wherein the plurality of nucleic acid molecules is a plurality of RNA molecules, said amplifying comprises reverse transcription using a primer that binds to the third primer binding site. In other embodiments, the nucleic acid molecules are DNA molecules, such as cDNA or genomic DNA. In a particular, preferred embodiment, the nucleic acid molecules comprise genomic DNA (gDNA). Preferably, said nucleic acid molecules comprising gDNA comprise gDNA fragments. In particular embodiments said gDNA is obtained from a patient in need of genome editing. In particular embodiments, wherein the plurality of nucleic acid molecules is a plurality of DNA molecules. Modified DNA molecules are transcribed into RNA using an RNA polymerase, where after DNA molecules are digested to enable selective amplification and sequencing of modified nucleic acid. Hence, in particular embodiments the adapter comprising a third primer binding site further comprises a DNA-dependent RNA polymerase promotor and said method further comprises, prior to said amplifying performing transcription of said one or more cleaved DNA molecules using said DNA-dependent RNA polymerase, resulting in one or more transcribed RNA molecules; and digesting DNA molecules, and wherein said amplifying comprises amplifying said one or more transcribed RNA molecules using primers that bind to said first or second primer binding site and to said third primer binding site. In particular embodiments, said amplifying comprises reverse transcription of said RNA molecules. Said digesting is advantageously performed using a DNase.

As describe herein above, the methods for enrichment of modified nucleic acid molecules, in particular wherein a strand break is induced, are advantageously used for enrichment prior to detection of modified nucleic acids. They are further particularly suitable for enrichments of nucleic acids wherein a strand break in induced for subsequent detection of off target activity of a targeted nuclease, for subsequent determination of cleavage efficiency of a targeted nuclease and for subsequent selection of a suitable guide RNA. Said method are advantageously performed in solution using enriched modified nucleic acid molecules prepared in accordance with the invention.

In one aspect the invention therefore provides a method for detecting a nucleic acid modification, comprising enriching one or more nucleic acid molecules wherein a nucleic acid modification is induced with a method according to the invention; and sequencing at least part of said amplified modified nucleic acid molecules. Advantageously said method is performed in solution.

In one aspect the invention provides a method for detecting a nucleic acid modification, comprising enriching one or more nucleic acid molecules wherein a nucleic acid modification is induced with a method according to the invention; sequencing at least part of said amplified modified nucleic acid molecules; and sequencing at least part of said amplified nucleic acid molecules that have not been modified. Advantageously said method is performed in solution.

In one aspect the invention provides a method for detecting off-target activity of a targeted nuclease specific for a selected target sequence, comprising enriching one or more nucleic acid molecules wherein a nucleic acid break is induced with a method according to the invention, wherein said agent comprises a targeted nuclease complex and detecting the presence of breaks in a sequence of said one or more nucleic acid molecules other than in said selected target sequence. Advantageously said method is performed in solution.

In one aspect the invention provides a method for determining cleavage efficiency of a targeted nuclease specific for a selected target sequence, comprising enriching one or more nucleic acid molecules wherein a nucleic acid break is induced with a method according to the invention, wherein said agent comprises a targeted nuclease complex; and determining a proportion of said plurality of nucleic acid molecules comprising a nucleic acid break at said selected target sequence. Advantageously said method is performed in solution.

In one aspect the invention provides a method for selecting a guide RNA from a plurality of guide RNAs specific for a selected target sequence, the method comprising enriching one or more nucleic acid molecules wherein one or more nucleic acid breaks are made with a method according to any one of claims 95-107 and 110-125, whereby said plurality of nucleic acid molecules is contacted with a plurality of RNA-guided nuclease complexes capable of inducing a nucleic acid break; and selecting a guide RNA based on location and/or amount of said nucleic acid breaks. Advantageously said method is performed in solution. In particular embodiments selecting comprises determining one or more locations in said one or more nucleic acid molecules comprising a break other than a location comprising said selected target sequence and selecting a guide RNA based on said one or more locations. In particular embodiments, selecting comprises determining a number of sites in said one or more immobilized nucleic acid molecules comprising a break other than a site comprising said selected target sequence and selecting a guide RNA based on said number of sites. A location in said one or more immobilized nucleic acid molecules comprising a break other than a location comprising said selected target sequence is herein also referred to as a location of an off-target break. Alternatively, said selecting comprises both determining the location of off-targets breaks and the number of locations of off-target breaks.

A modification that is induced in the methods of the invention for enrichment of modified nucleic acid molecules is preferably selected from the group consisting of an insertion, a replacement, a strand break and a recombination. In particular embodiments, the agent capable of inducing the modification is a chemical agent. Examples of such chemical agents include, but are not limited to, etoposide and teniposide. In particular embodiments, the agent capable of inducing the modification is an enzyme. Non-limiting examples of such enzymes are a nuclease, a (viral) integrase, a recombinases, a transposase, an argonaute. In a preferred embodiment, said enzyme comprises a nuclease.

In a particularly preferred embodiment, said agent comprises a targeted nuclease complex. Preferably the nucleic acid modification is a strand break, more preferably a SSB, a DSB or a nick, most preferably a DSB, and the agent comprises a nuclease, more preferably a targeted nuclease. In particular embodiments, said targeted nuclease complex comprises a ZFN, TALEN or CRISPR-Cas. In one embodiment said targeted nuclease complex comprises a RNA-directed nuclease complex. In one embodiment the targeted nuclease complex or the RNA-guided nuclease complex is a non-naturally occurring or engineered complex. In a preferred embodiment, said nuclease is selected from the group consisting of Cas9, Cpf1, C2c1, C2c2, C2c3, a group 29 nuclease, a group 30 nuclease and derivatives thereof. In a preferred embodiment, said targeted nuclease complex is a CRIPSR-Cas complex.

In particular embodiments, the modification comprises an insertion or a replacement. In particular embodiments, the modification comprises insertion of the adapter comprising the third binding site into the nucleic acid molecules or, alternatively, replacement of one or more nucleotides in the nucleic acid molecule with the adapter comprising the third binding site. Such methods allow for selective amplification and detection of modified nucleic acid molecules.

In particular embodiments of the enrichment methods of the invention, wherein said nucleic acid modification comprises a DSB which results in an overhang, said DSB is blunt ended before attaching to said adapter comprising a primer binding site.

In particular embodiments of the enrichment methods of the invention, one ore more of the adapter comprising a primer binding site, such as the adapter comprising a third primer binding site, further comprises an adenine-tail. Such adenine-tail suitably prevents formation of adapter dimers or concatamers of adapters.

In particular embodiments of the enrichment methods of the invention, one or more of the adapters comprise a unique molecular identifier (UMI) and/or a barcode. It is preferred that one or both of the adapters comprising a first primer binding site or a second primer binding site further comprise a unique molecular identifier and/or a barcode.

In a further aspect, the invention provides a method for detecting a nucleic acid break, comprising contacting a plurality of nucleic acid molecules flanked by adapters comprising a ligation-blocking moiety with an agent capable of inducing a nucleic acid break, resulting in one or more cleaved nucleic acid molecules; attaching an adapter comprising a primer binding site to said one or more cleaved nucleic acid molecules; sequencing at least part of said one or more cleaved nucleic acid molecules using a primer specifically binding to said primer binding site, said part comprising said nucleic acid modification.

In certain example embodiments, nucleic acid molecules to be assayed for off target effects may further be processed through one or more enrichment steps to reduce noise in the final output signal. In one example embodiment this comprises labeling the ends of at least a portion of nucleic acid molecules in a plurality of nucleic acid molecules with adapters on both the 5′ and 3′ adapters. The adapters may be the same as those described above but further modified to render the adapters digestion resistant. In certain example embodiments, this may comprise modifying the ends of the adapters to incorporate certain chemical modifications that render nucleic acid molecules labeled with the modified adapters nuclease digestion resistant. In certain example embodiments, the adapters incorporate phosphorothioate bonds on at least the ends of the adapters to render nucleic acid molecules labeled on both the 5′ and 3′ ends resistant to digestion. Accordingly, only the portion of nucleic acid molecules successfully labeled on both the 5′ and 3′ ends with the adapters will be digestion resistant. To enrich for nucleic acid molecules properly labeled on both ends with the adapters, a digestion step may be included that will remove any non-labeled or single labeled nucleic acid fragments. The doubly end protected nucleic acid molecules may then be manipulated and tested for the effects of said manipulation using those methods further described herein.

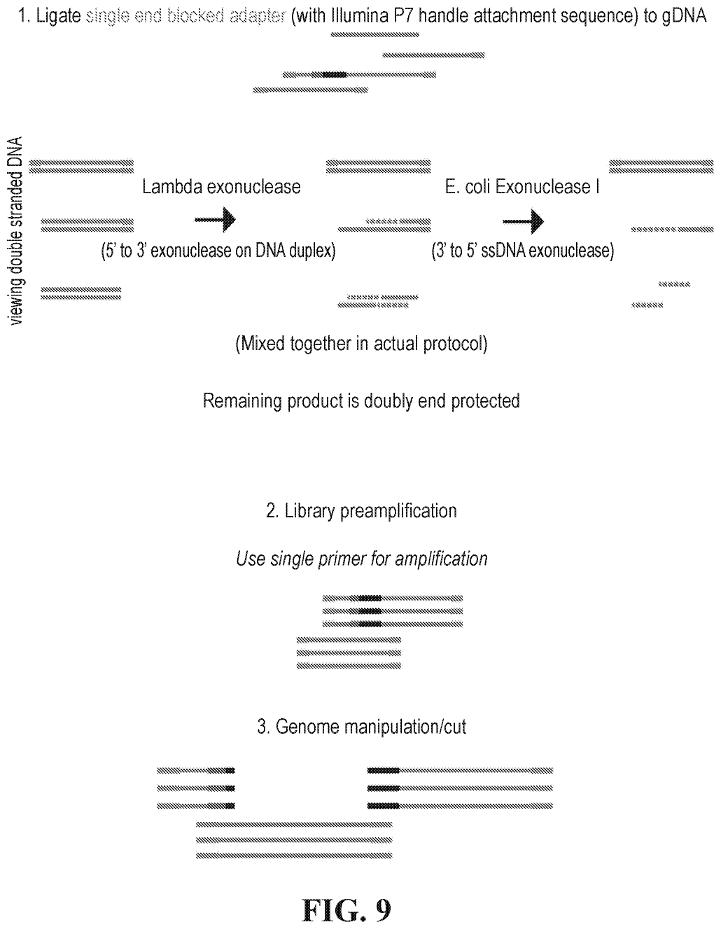

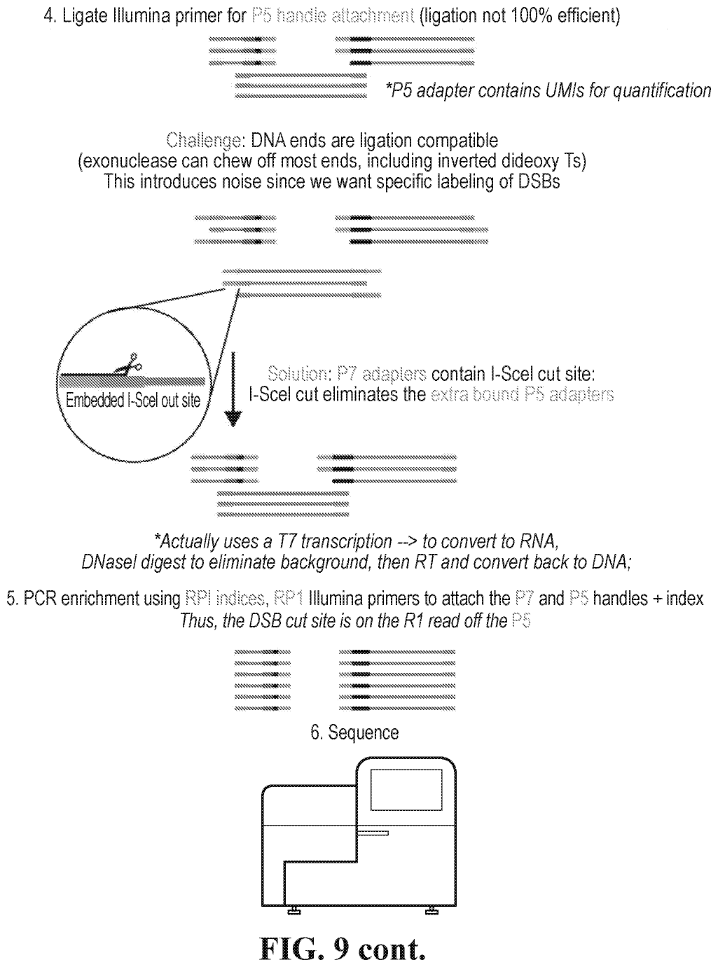

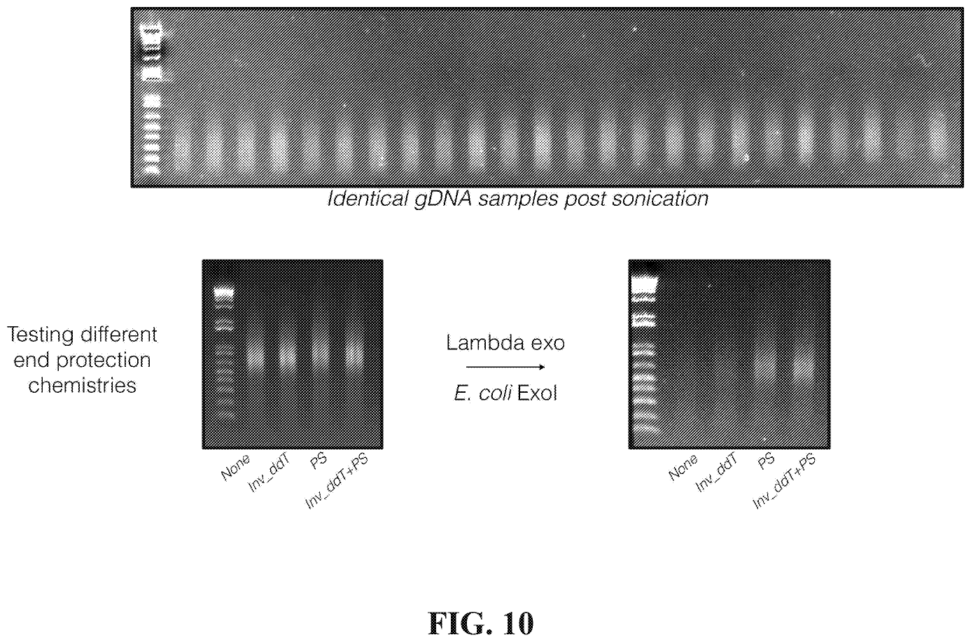

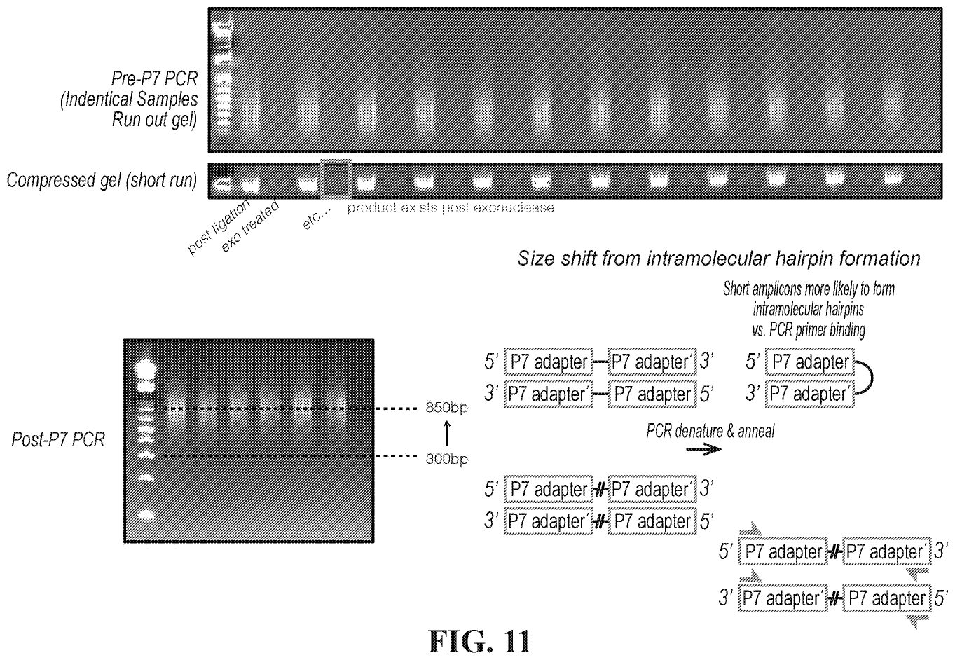

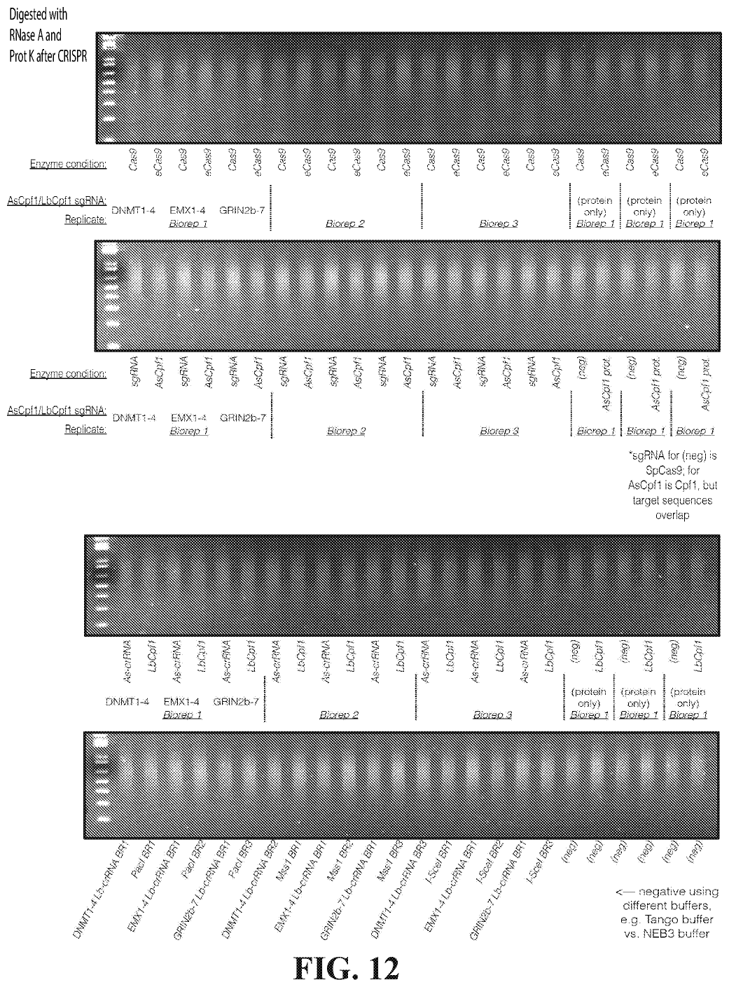

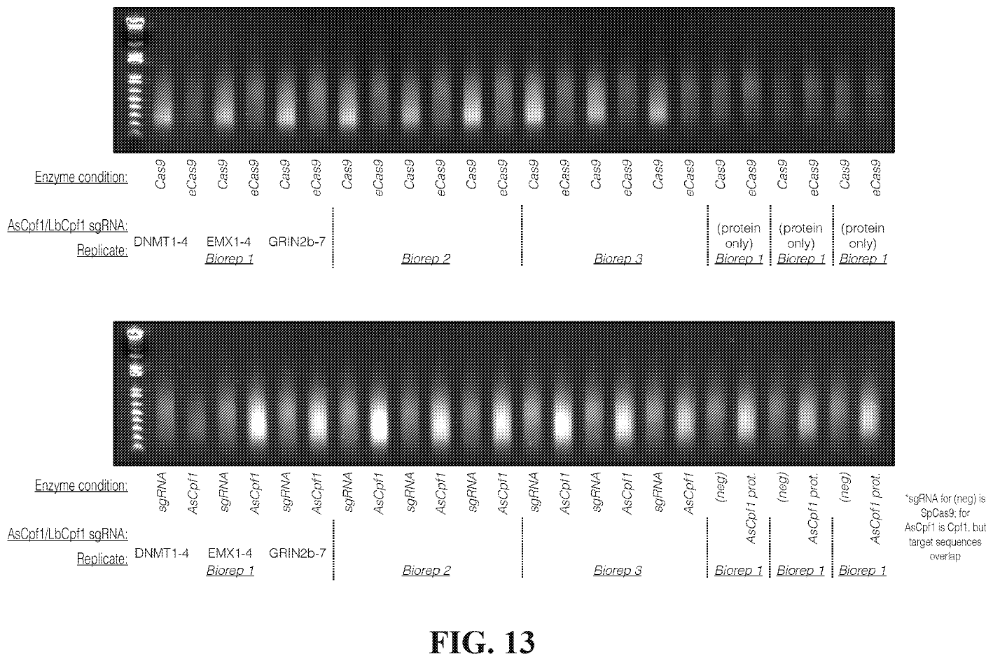

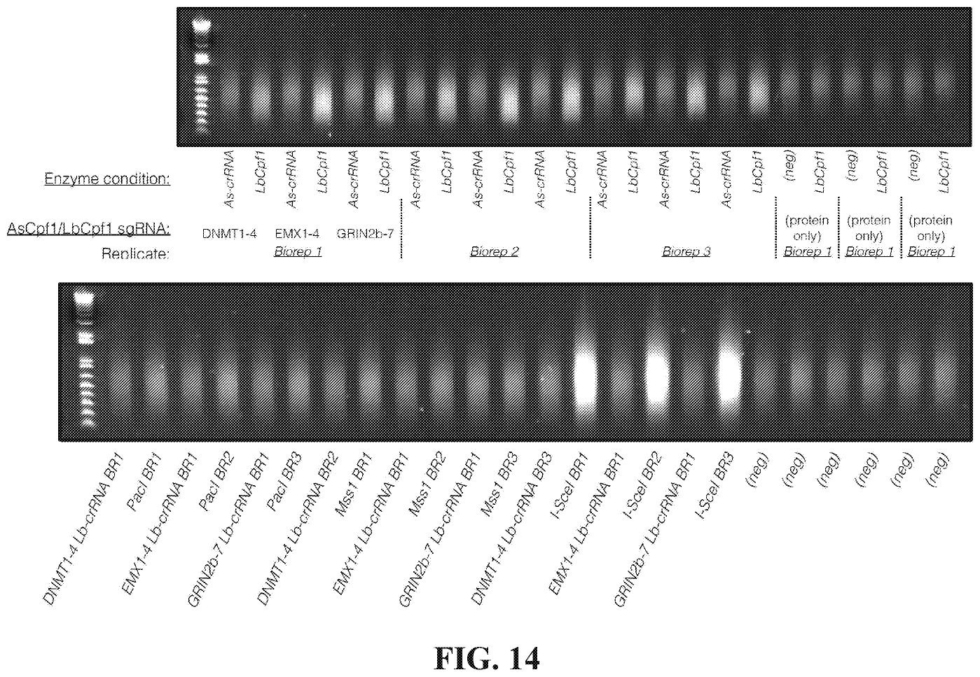

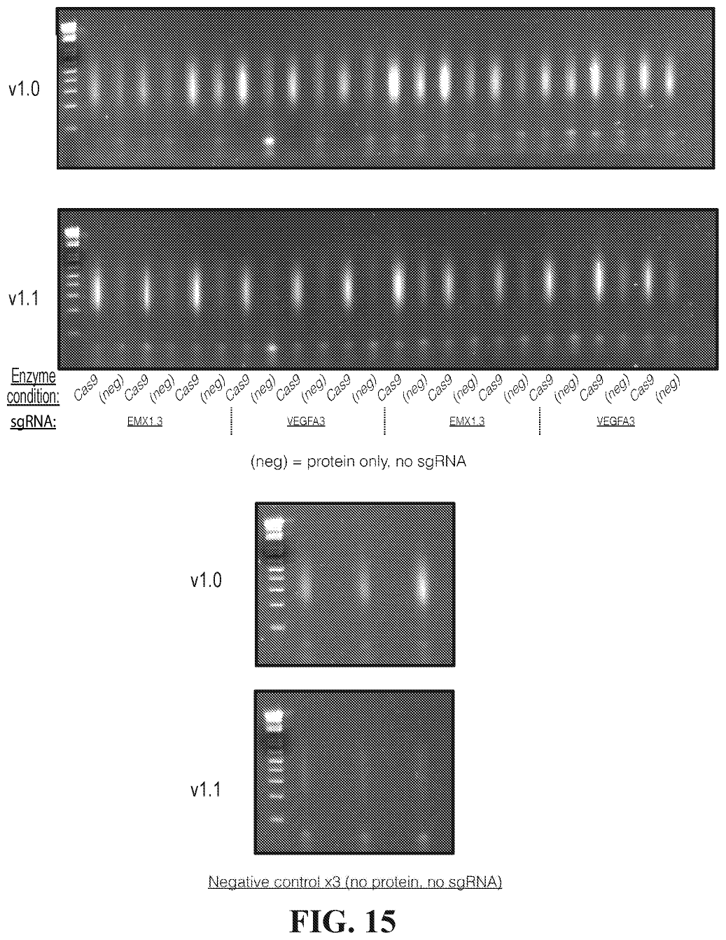

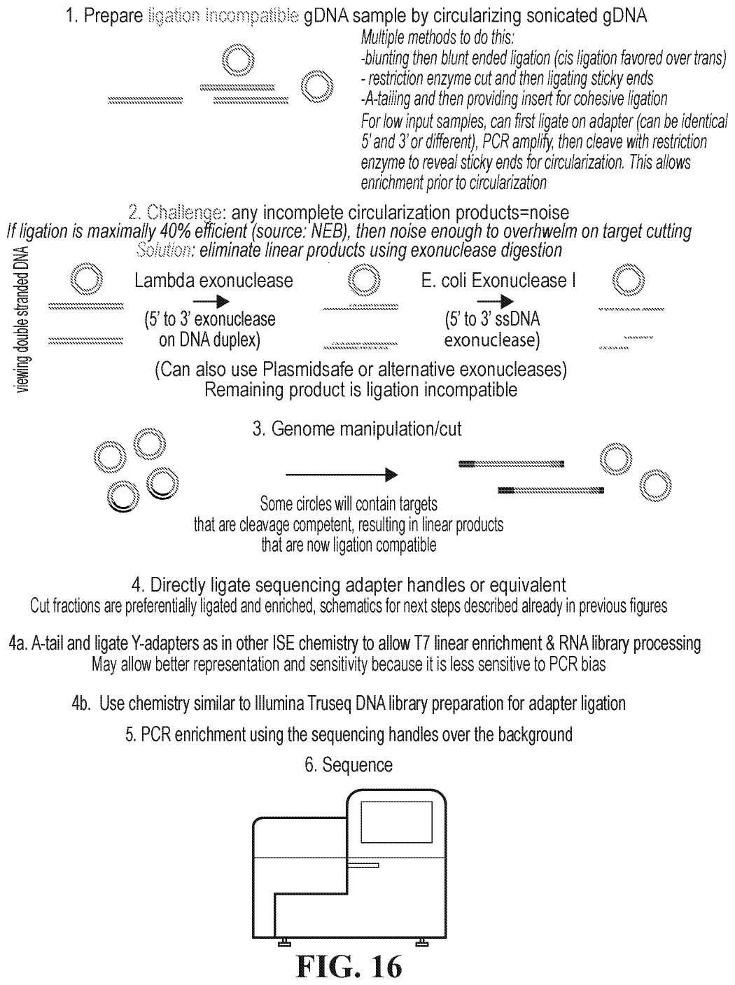

In certain example embodiments, the adapter further used to end-label the nucleic acid molecules may further be configured to include one or more cleavage sites. When the second adapter is attached after manipulation of the DNA as described herein, the possibility exists that the second adapter may ligate to the existing adapters on the terminal ends of the nucleic acid molecule rather than the ended sites newly created by manipulation of the nucleic acid. See FIG. 9. The cleavage site may be a restriction site or any other labile bond that allows the end of the initial adapter to be removed and thus removing any unwanted ligation products. After the unwanted ligation products are removed, the methods may proceed with the further enrichment and sequencing steps disclosed herein. In certain example embodiments, the nucleic acid molecules labeled with the sequencing adapters may first undergo a RNA transcription step, e.g. T7 transcription, to convert to RNA, followed by a DNAase digestion step to further eliminate background, the reverse transcription and conversion back to DNA for further processing. In certain example embodiments, the RNA transcription step may be facilitated by A-tailing and/or use of Y adapters.

In certain example embodiments, in solution enrichment steps may comprise shearing the nucleic acid molecules and then circularizing the sheared nucleic molecules. The circularized nucleic acid molecules thus become ligation incompatible and resistant to digestion. Multiple methods may be utilized to induce circularization of the nucleic acids including, but not limited to, blunting followed by blunt end ligation with cis ligation being preferred over trans, cutting with a restriction enzyme and then ligation sticky ends, or A-tailing and then providing insert for cohesive ligation. For low input samples, the method may further comprise first ligating on adapters to the 5′ and 3′ ends (can be identical 5′ and 3′ or different), PCR amplification, then cleavage with a restriction enzyme to generate sticky ends for circularization. Nucleic acid molecules that are not circularized may then be removed by digestion. Any suitable nuclease may be used for digestion of non-circularized nucleic acids. At least a portion of the circularized nucleic acids will contain targets that are cleavage competent resulting in linear products that are now ligation competent. Sequencing adapters may then be ligated directly to the linear product. In certain example embodiments, A-tail and ligation of Y-adapters may also be used to allow for RNA linear enrichment and RNA library processing as described elsewhere in this application. A DNase digestion step may be applied to further remove background followed by a reverse transcription step to convert back to DNA. Further PCR enrichment and sequencing may be carried out as described elsewhere in the application.

In particular embodiments, the modification comprises an insertion or a replacement. In particular embodiments, the modification comprises insertion of the adapter comprising the third binding site into the nucleic acid molecules or, alternatively, replacement of one or more nucleotides in the nucleic acid molecule with the adapter comprising the third binding site. Such methods allow for selective amplification and detection of modified nucleic acid molecules.

In particular embodiments of the enrichment methods of the invention, wherein said nucleic acid modification comprises a DSB which results in an overhang, said DSB is blunt ended before attaching to said adapter comprising a primer binding site.

In particular embodiments of the enrichment methods of the invention, one ore more of the adapter comprising a primer binding site, such as the adapter comprising a third primer binding site, further comprises an adenine-tail. Such adenine-tail suitably prevents formation of adapter dimers or concatamers of adapters.

In particular embodiments of the enrichment methods of the invention, one or more of the adapters comprise a unique molecular identifier (UMI) and/or a barcode. It is preferred that one or both of the adapters comprising a first primer binding site or a second primer binding site further comprise a unique molecular identifier and/or a barcode.

In a further aspect, the invention provides a method for detecting a nucleic acid break, comprising contacting a plurality of nucleic acid molecules flanked by adapters comprising a ligation-blocking moiety with an agent capable of inducing a nucleic acid break, resulting in one or more cleaved nucleic acid molecules; attaching an adapter comprising a primer binding site to said one or more cleaved nucleic acid molecules; sequencing at least part of said one or more cleaved nucleic acid molecules using a primer specifically binding to said primer binding site, said part comprising said nucleic acid modification.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridising to the reference sequence under highly stringent conditions. Generally, in order to maximize the hybridization rate, relatively low-stringency hybridization conditions are selected: about 20 to 25° C. lower than the thermal melting point (Tₘ). The Tₘ is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tₘ. In order to require at least about 70% nucleotide complementarity of hybridized sequences, moderately-stringent washing conditions are selected to be about 15 to 30° C. lower than the Tₘ. Highly permissive (very low stringency) washing conditions may be as low as 50° C. below the Tₘ, allowing a high level of mis-matching between hybridized sequences. Those skilled in the art will recognize that other physical and chemical parameters in the hybridization and wash stages can also be altered to affect the outcome of a detectable hybridization signal from a specific level of homology between target and probe sequences.

The terms “polynucleotide”, “nucleic acid”, “nucleic acid sequence”, “nucleotide”, “nucleotide sequence” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of nucleic acid sequences: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers.

A used herein the term “nucleic acid modification” refers to any nucleic acid modification that can be detected by sequencing of a nucleic acid molecule wherein said modification is induced. Any such modifications can be detected using the methods of the invention. A skilled person is well aware of nucleic acid modifications that can be detected by sequencing of nucleic acid wherein the modification is induced. In particular embodiments, the nucleic acid modification is selected from the group consisting of methylation, a mutation, a deletion, an insertion, a replacement, a ligation, a digestion, a break and a recombination. In a preferred embodiment, the modification is the introduction of a strand break. Preferably, said nucleic acid modification is a break, more preferably a nick, a single strand break (SSB) or a double strand break (DSB). If said nucleic acid modification is a nick in double stranded nucleic acid molecules, the methods my further comprise contacting said one or more immobilized nucleic acid molecules with an S1 nuclease subsequent to contacting with an agent capable of inducing a nick. Preferably, contacting said immobilized nucleic acid molecule with said agent capable of inducing a nucleic acid modification, preferably a targeted nuclease complex is for a period of time sufficient for agent to induce the modification, preferably for the nuclease to induce DSBs. Contacting the immobilized nucleic acid molecules with a nuclease or targeted nuclease complex will result in cleavage of those molecules that comprise one or more target sites that can be cleaved by the nuclease.

Nucleic acid molecules may be isolated from biological samples containing a variety of other components, such as proteins, lipids and non-template nucleic acids. Nucleic acid molecules may be obtained from any cellular material, obtained from an animal, plant, bacterium, fungus, or any other cellular organism. The nucleic acid molecules, preferably gDNA, are preferably derived from a one or more cells. The cells may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish or shrimp. The cell may also be a plant cell. The plant cell may be of a crop plant such as cassava, corn, sorghum, wheat, or rice. The plant cell may also be of an algae, tree or vegetable. In a preferred embodiment the cell is a human cell. In certain embodiments, the nucleic acid molecules may be obtained from a single cell. In certain embodiment, genomic DNA is obtained from a single cell. Nucleic acid molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. A sample may also be total RNA extracted from a biological specimen, a cDNA library, viral, or genomic DNA.

Generally, nucleic acid may be extracted from a biological sample by a variety of techniques such as those described by Maniatis, et al., Molecular Cloning: A Laboratory Manual, Cold Spring Harbor, N.Y., pp. 280-281 (1982). Reference is made to WO 2016040476 for methods to isolate, lyse, optionally barcode, and prepare nucleic acids from single cells, and which is incorporated herein by reference.

In a preferred embodiment, the gDNA is obtained from a cell or cells of a patient in need of genome editing. The terms “individual” or “patient” as used herein refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline. As used herein “genome editing” as used herein refers to changing a gene. It may include, but is not limited to, correcting or restoring a mutant gene and knocking out a gene, such as a mutant gene or a normal gene. Genome editing may be used to treat a disease. As used herein, a patient in need of genome editing refers to an individual who could have therapeutic benefit from genome editing.

Nucleic acid molecules immobilized on a solid support and used in the methods of the invention are preferably less then 2000 bp, preferably less then 1500 bp, preferably less than 1000 bp, such as 400-800 bp, 400-600 bp, 400-500 bp or 300-400 bp, if double stranded. Alternatively, said nucleic acid molecules are single stranded and less then 2000 nucleotides, preferably less then 1500 nucleotides, preferably less than 1000 nucleotides, such as 400-800 nucleotides, 400-600 nucleotides, 400-600 nucleotides or 300-400 nucleotides. In one embodiment, nucleic acid molecules immobilized on a solid support comprise genomic DNA (gDNA) fragments or cDNA. Genomic DNA is preferably randomly fragmented. It is preferred that the nucleic acid molecules comprise a library of gDNA comprising fragments of the entire genome of a cell. If double stranded, said gDNA fragments are preferably of less then 2000 bp, preferably less then 1500 bp, preferably less than 1000 bp, such as 400-800 bp, 400-600 bp, 400-500 bp or 300-400 bp. Alternatively, said gDNA fragments are single stranded and less then 2000 nucleotides, preferably less then 1500 nucleotides, preferably less than 1000 nucleotides, such as 400-800 nucleotides, 400-600 nucleotides, 400-500 nucleotides or 300-400 nucleotides. Nucleic acid molecules, in particular gDNA, can be fragmented using any method known in the art. This can comprise, without limitation, sonication, endonuclease digestion, limited restriction enzyme digestion, or tagmentation. “Tagmentation” combines fragmention and adapter ligation in a single step that greatly increase the efficiency of the library preparation method.

A method of the invention preferably involves a plurality of immobilized nucleic acid molecules. As used herein a plurality of immobilized nucleic acid molecules means at least two immobilized nucleic acid molecules. Preferably it refers to at least 5, at least 10, at least 15, at least 20, at least 25, at least 50, at least 75 immobilized nucleic acids. Preferably the one or more immobilized nucleic acid molecules is one or more clusters of immobilized nucleic acid molecules. Similarly, a plurality of immobilized nucleic acid molecules preferably is a plurality of clusters of immobilized nucleic acid molecules. As used herein a cluster of nucleic acid molecules refers to multiple copies of the same nucleic acid molecules, for instance obtained by amplification of a single nucleic acid molecule. Each cluster may contain hundreds to one million or more copies of the original nucleic acid molecule. Hence, a plurality of immobilized nucleic acid molecules preferably means at least two clusters of immobilized nucleic acid molecules. Preferably it refers to at least 5, at least 10, at least 15, at least 20, at least 25, at least 50, at least 75 clusters of immobilized nucleic acids. The methods of the invention are particularly suitable for detecting nucleic acid modifications such as strand breaks in an entire genome of a cell. Hence, in a preferred embodiment the plurality of nucleic acid molecules or clusters thereof immobilized on a solid support used in the methods of the invention comprises an entire genome of a single cell in fragmented form. For instance, in contains the entire genome of a cell from a patient in need of genome editing.

The methods of the invention use nucleic acid molecules immobilized on a solid support. “Solid support” as used herein refers to any solid surface to which nucleic acids can be attached. Hence, any solid support suitable for attaching nucleic acid molecules can be used for this purpose. Preferred, but non limiting, examples are a chip, an array, a flow cell, a microwell and a bead. Solids supports may for instance comprise latex (e.g. in case of beads), dextran (e.g. in case of beads), polystyrene surfaces, polypropylene surfaces, polyacrylamide gel, gold surfaces and glass surfaces. In a preferred embodiment, the solid support comprises a glass or a polystyrene surface. Any such solid support optionally comprises an affinity treated surface. An “affinity treated surface” as used herein refers to the support comprising an inert substrate or matrix which has been functionalized by the presence of a layer or coating comprising reactive groups that allow covalent attachment to nucleic acid molecules. In one embodiment, the solid support is a flow cell such as an Illumina flow cell. An Illumina flow cell comprises an 8-channel sealed glass microfabricated device. In another embodiment the solid support comprises beads, such as immobilized affinity bead, e.g. biotinylated beads. For instance, the bead is linked to chemical groups (such as biotin) that can bind to a chemical groups (such as streptavidin) present on the template nucleic acid molecules.

The term “immobilized” as used herein encompasses direct and indirect attachment to a solid support via covalent or non-covalent bonds. In preferred embodiments of the invention, covalent attachment is used. Nucleic acid fragment, both larger nucleic acid molecules, such as molecules over 300 nucleotides long, polynucleotides, e.g. primers and adapters as described herein, can be linked to a solid support in a covalent manner by physical, chemical or biological means. Hence, in particular embodiments, nucleic acid molecules comprise an adapter comprising a protein or chemical moieties for binding to protein or chemical moieties that are immobilized on a solid support are used for immobilization to a solid support. Suitable methods comprising the use of specific groups for immobilization on a solid support are described in Adessi C et al., Nucleic Acids Res 2000, 28(20):E87 and Okamoto T et al., Nat Biotechnol 2000, 18(4): 438-41.

Typically oligonucleotides to be used for attachment of nucleic acid fragments, e.g. gDNA or cDNA, are immobilized such that at least a portion of the sequence of the oligonucleotide is capable of hybridizing to a complementary sequence in the nucleic acid fragments. Hence, immobilization of nucleic acid fragments can occur via hybridization to a surface attached oligonucleotide.

Nucleic acid molecules may be modified or processed before being used in methods of the invention using standard genetic engineering techniques, for instance by addition of adapter sequences comprising a sequence that is able to hybridize to immobilized polynucleotides as described herein. Such techniques are for instance described in WO 00/18957, which is incorporated herein by reference. If the nucleic acid molecules are RNA, such as mRNA, it can be transcribed into cDNA using a reverse transcriptase and optionally converted into double stranded DNA before being immobilized on a solid support for use in the methods of the invention. Hence, in a preferred embodiment a method of the invention comprises allowing one or more nucleic acid molecules flanked by a first and a second adapter to hybridize to one of a plurality of first or second oligonucleotides that are immobilized on a solid support, whereby said first adapter comprises a sequence that is able to hybridize to said first immobilized oligonucleotides and said second adapter comprises a sequence that is complementary to a sequence that is able to hybridize to said immobilized second oligonucleotides.

The selected target sequence can be any nucleotide sequence. For example, the target polynucleotide can be a polynucleotide residing in the nucleus of the eukaryotic cell, preferably a human cell. The target sequence is preferably a therapeutically relevant sequence, i.e. a potential target for therapeutic intervention. As used herein the term “selected target sequence” refers to a nucleic acid sequence comprising a target site of a given nuclease. Hence, said target sequence is preferably selected to be subjected to a strand break induced by a targeted nuclease. The selected target sequence can refer to the specific nucleic acid sequence that is targeted by the nuclease, such as the sequence that is targeted by a guide RNA. Alternatively, the selected target sequence may refer to a larger nucleic acid sequence such as a sequence coding a gene product (e.g., a protein) or a non-coding sequence (e.g., a regulatory polynucleotide or a junk DNA).

The target sequence can be a control element or a regulatory element or a promoter or an enhancer or a silencer. The promoter may, in some embodiments, be in the region of +200 bp or even +1000 bp from the TTS. In some embodiments, the regulatory region may be an enhancer. The enhancer is typically more than +1000 bp from the TTS. More in particular, expression of eukaryotic protein-coding genes generally is regulated through multiple cis-acting transcription-control regions. Some control elements are located close to the start site (promoter-proximal elements), whereas others lie more distant (enhancers and silencers) Promoters determine the site of transcription initiation and direct binding of RNA polymerase II. Three types of promoter sequences have been identified in eukaryotic DNA. The TATA box, the most common, is prevalent in rapidly transcribed genes. Initiator promoters infrequently are found in some genes, and CpG islands are characteristic of transcribed genes. Promoter-proximal elements occur within ≈200 base pairs of the start site. Several such elements, containing up to ≈20 base pairs, may help regulate a particular gene. Enhancers, which are usually ≈100-200 base pairs in length, contain multiple 8- to 20-bp control elements. They may be located from 200 base pairs to tens of kilobases upstream or downstream from a promoter, within an intron, or downstream from the final exon of a gene. Promoter-proximal elements and enhancers may be cell-type specific, functioning only in specific differentiated cell types. However, any of these regions can be the target sequence and are encompassed by the concept that the target can be a control element or a regulatory element or a promoter or an enhancer or a silencer.

With reference to the methods of the invention, “amplification” as used herein means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. a preferred polymerase is T7 polymerase. Amplification may be carried out by any method known in the art for amplification of immobilized nucleic acid molecules. Example nucleic acid amplification reactions that may be used include PCR, RT-PCR (for RNA), whole genome amplification (WGA), loop-mediated isothermal amplification (LAMP), linear amplification, rolling circle amplification, strand displacement amplification or other nucleic acid amplification reactions known in the art, and combinations of these amplification methods. A preferred amplification method is bridge amplification. The term “bridge amplification” as used herein refers to any amplification reaction that allows the generation of in situ copies of a specific nucleic acid molecule attached to a solid support. For example, bridge amplification is performed to produce DNA molecules that are compatible with an Illumina sequencing techniques. Bridge amplification involves clonal amplification, wherein the cloned fragments are amplified using primers that are attached to a solid surface or bind to a primer binding site attached to a solid surface. Such configurations are compatible with an Illumina flow cell and Illumina Genome Analyzer. For example, DNA molecules are physically bound to the surface of the solid support such that they may be sequenced in parallel. Hence, in a preferred embodiment, a solid support used in the methods of the invention is a flow cell. A preferred flow cell is described herein elsewhere.

In particular embodiments of the invention, nucleic acid molecules are amplified prior to immobilization thereof on a solid support. In particular embodiments of the invention, nucleic acid molecules are amplified by emulsion or droplet amplification. For instance, such methods are particularly suitable if the nucleic acid molecules are immobilized on a chip or on beads. In such methods, amplification is for instance performed by emulsion amplification so that for each original nucleic acid molecule a cluster of multiple copies thereof is obtained, which can subsequently be immobilized on a solid support, such as a chip, flow cell or beads. In particular embodiment, emulsion amplification is performed in droplets. In further embodiments, emulsion amplification is performed by attaching nucleic acid molecules to be amplified to a bead. The bead is for instance linked to a large number of a single primers that are complementary to a primer binding site in the nucleic acid molecule and amplified copies thereof. In particular embodiments, the bead is linked to chemical groups (e.g., biotin) that can bind to chemical groups (e.g., streptavidin) included on the template nucleic acid molecules and amplified copies thereof. The beads may be suspended in aqueous reaction mixture and then encapsulated in a water-in-oil emulsion. Hence, the template nucleic acid molecule is for instance bound to the bead prior to emulsification, or the template nucleic acid molecule is included in solution in the reaction mixture for amplification.

In certain embodiments, amplification comprises use of Taq polymerase. For example, a Taq polymerase can be used with a single primer and provide linear amplification. In certain embodiments, the linear amplification comprises transcription from a T7 adapter by T7 polymerase. The T7 adapter can comprise a unique molecular identifier (UMI) and barcode sequences and includes a T7 polymerase binding site for linear amplification of captured inserts and flanking DNA. In certain embodiments where adaptors are used, there is no requirement for ligation of second “distal” adapters to enable amplification. In certain embodiments, the invention provides unique molecular identifiers (UMIs). In certain embodiments, transfection and processing in the same 24-well plate allows for high throughput while maximizing the number of cells and minimizing non-specific DSBs. In particular embodiments, the amplification product is a transcription product and is an RNA. In another embodiment, the linear amplification product is extended from a primer by a DNA polymerase and is a DNA. In certain embodiments, RNA products, for example which can be repeatedly transcribed from a T7 transcription sequence by T7 polymerase in a single reaction step, are preferred.

To determine the sequence or the nucleic acid comprising the modification, such as a strand break, the amplification product is then sequenced. Sequencing of nucleic acid molecules immobilized on a solid support can be performed using any method known in the art for sequencing including, but not limited to, sequencing by synthesis and ion semiconductor sequencing. Ion semiconductor sequencing based on the detection of hydrogen ions that are released during the polymerization of DNA. Sequencing by synthesis techniques (i.e., for example, dye-termination electrophoretic sequencing) uses a DNA polymerase to determine the base sequence. Alternatively, a reversible terminator method may be used wherein fluorescently labeled nucleotides are individually added, such that each position is determined in real time (i.e., for example, Illumina). A blocking group on each labeled nucleotide is then removed to allow polymerization of another nucleotide. Massively parallel sequencing of millions of fragments has been successfully commercialized by a reversible terminator-based sequencing chemistry (Illumina). For example, the Illumina sequencing technology relies on the attachment of randomly fragmented (genomic) DNA to a planar, optically transparent surface. These attached DNA fragments are extended and bridge amplified to create an ultra-high density sequencing flow cell with hundreds or millions of clusters, each containing copies of the same DNA template. These templates are sequenced using a robust four-color DNA sequencing-by-synthesis technology that employs reversible terminators with removable fluorescent dyes. This approach ensures high accuracy and true base-by-base sequencing, eliminating sequence-context specific errors and enabling sequencing through homopolymers and repetitive sequences. High-sensitivity fluorescence detection may be achieved using laser excitation and total internal reflection optics. Sequence reads are aligned against a reference genome and genetic differences are called using specially developed data analysis pipeline software.

After completion of the first read, the nucleic acid templates can be regenerated in situ to enable a second read from the opposite end of the fragments. A paired-end module directs the regeneration and amplification operations to prepare the templates for the second round of sequencing. First, the newly sequenced strands are stripped off and the complementary strands are bridge amplified to form clusters. Once the original templates are cleaved and removed, the reverse strands undergo sequencing-by-synthesis. The second round of sequencing occurs at the opposite end of the templates.

A single molecule amplification step compatible with the Illumina Genome Analyzer may start with an Illumina-specific adapter library and takes place on an oligo-derivatized surface of a flow cell. An Illumina flow cell comprises an 8-channel sealed glass microfabricated device that allows bridge amplification of fragments on its surface, and uses DNA polymerase to produce multiple DNA copies (i.e., for example, DNA clusters) wherein each cluster represents a single molecule that initiated the cluster amplification. A separate library can be added to each of the eight channels, or the same library can be used in all eight, or combinations thereof. Each cluster may contain hundreds to a million amplicons (e.g., copies) of the original fragment, which is sufficient for reporting incorporated bases at the required signal intensity for detection during sequencing.

In some embodiments, for example, sequencing includes that each nucleotide type (e.g. single nucleotide, oligonucleotide, etc.) is tagged with a fluorescent tag (e.g. dye, pigment, or other optical label or tag, e.g. as described herein) that permits analysis of the nucleotide added or otherwise detected at a particular site to be determined by analysis of optical image data. These tags may then be removed by cleaving the tags in a separate step, or may be removed by natural processes (e.g. by attaching the tag to a phosphate of the nucleotide that gets removed by action of the polymerase adding an additional nucleotide). In some embodiments, as with fluorescent labels, the labels may be optical labels. In other embodiments, the labels may be non-optical labels (e.g. may be labels that change an electrical characteristic detectable by a detection circuit).

Second-generation sequencing instruments can determine one hundred million or more short sequences per run. The Illumina Genome Analyzer builds millions of distinct clusters on a flow cell, each consisting of several hundred identical DNA molecules. The Illumina system utilizes a sequencing-by-synthesis approach in which all four nucleotides are added simultaneously to the flow cell channels, along with DNA polymerase, for incorporation into the oligo-primed cluster fragments. Specifically, the nucleotides carry a base-unique fluorescent label and the 3 ‘—OH group is chemically blocked such that each incorporation is a unique event. An imaging step follows each base incorporation step. After each imaging step, the 3’ blocking group is chemically removed to prepare each strand for the next incorporation by DNA polymerase. This series of steps continues for a specific number of cycles, as determined by user-defined instrument settings. In other embodiments, such as pyrosequencing, nucleotides may be added without (fluorescent) labels and sequencing is based on detecting the pyrophosphate that is released during the extension process of the polymerase. In some embodiments of pyrosequencing, the pyrophosphate is used in a light generating reaction (e.g. is converted to ATP and is detected using luciferase) and is subject to optical detection. In some embodiments of pyrosequencing, the pyrophosphate is used in an electronic detection step (e.g. is converted by phosphoric acid which changes a current detectable by detection circuitry such as a detection electrode).

Reference is made to reference is for instance made to U.S. Pat. No. 7,972,820, US 2009/0226975, WO 06/064199, WO 07/01025, WO 98/44151, WO 02/46456, WO 00/18957, WO 2013/117595/EP1591541, U.S. Pat. Nos. 8,993,271, 8,143,008, 7,985,565, 8,476,044, 6,355,431, which are incorporated herein by reference, for describing suitable methods for amplification of nucleic acid molecules immobilized on a solid support and sequencing immobilized nucleic acid molecules.

In preferred embodiments of the invention, determining the off-target activity of a targeted nuclease, such as a Cas protein, may allow an end user or a customer to predict the best cutting sites in a genomic locus of interest. In a further embodiment of the invention, one may obtain a ranking of cutting frequencies at various putative off-target sites to verify in vitro, in vivo or ex vivo if one or more of the worst case scenario of non-specific cutting does or does not occur. In another embodiment of the invention, the determination of off-target activity may assist with selection of specific sites of an end user or customer is interested in maximizing the difference between on-target cutting frequency and the highest cutting frequency obtained in the ranking of off-target sites. Another aspect of selection includes reviewing the ranking of sites and identifying the genetic loci of the non-specific targets to ensure that a specific target site selected has the appropriate difference in cutting frequency from targets that may encode for oncogenes or other genetic loci of interest. Aspects of the invention may include methods of minimizing therapeutic risk by verifying the off-target activity of the CRISPR-Cas complex. Further aspects of the invention may include utilizing information on off-target activity of the CRSIPR-Cas complex to create specific model systems (e.g. mouse) and cell lines. The methods of the invention allow for rapid analysis of non-specific effects and may increase the efficiency of laboratory analysis.

In some embodiments, a method is provided that comprises providing a plurality of candidate targeted nuclease complexes that are designed or known to cut the same target sequence and analyzing the sites actually cleaved by each complex. For instance, a plurality of complexes comprising the same targeted nuclease but different guide RNAs can be analyzed. Thereby any cleaved off-target sites can be detected and candidate complexes or guide RNAs can be selected based on the detected off-target. In some embodiments, a method of the invention is used to select the most specific guide RNA, and consequently most specific targeted nuclease complex, from a plurality of candidate guide RNAs. For example, the targeted nuclease complex that cleaves the selected target sequence with the highest specificity, the complex that cleaves with the highest efficiency, the complex that cleaves the lowest number of off target sites, the complex that does not cleave any target site other than the target sequence. In some embodiments, a guide RNA or targeted nuclease complex is selected that does not cleave off target sites in the genome of a patient in need of genomic editing at the therapeutically effective concentration of the targeted nuclease complex. As used herein “off-target sites” or “off-target activity” refers to cleavage of sites that differ from the selected target site sequence. In certain embodiments, the cleavage efficiency is analyzed by determining the proportion of immobilized nucleic acid molecules that comprise a nucleic acid break at the selected target sequence. Preferably said proportion is the proportion of immobilized nucleic acid molecules that comprise a strand break at the selected target sequence and the immobilized nucleic acid molecules that do not comprise a strand break at an off-target site. In some embodiments, determining cleavage efficiency is performed by sequencing at least part of said one or more immobilized nucleic acid molecules comprising a nucleic acid break using a primer specifically binding to a primer binding site on the immobilized nucleic acids. In some embodiments, determining cleavage efficiency is performed by determining fluorescence intensity of said one or more immobilized nucleic acid molecules. This can for instance be achieved by attaching an adapter to the cleaved nucleic acid molecules that comprises a fluorescent moiety. In one embodiment, the period of time that is needed to achieve a particular intensity of the fluorescent signal is used as a measure for cleavage efficiency. Indeed, high cleavage efficiency of a nuclease or complex results in a quick increase in fluorescent signal. In one embodiment, said fluorescence intensity is determined cyclically. In such methods, short pulses of nuclease activity are repeated. For instance, each cycle comprises addition of a selected amount of the targeted nuclease complex to the plurality of nucleic acid molecules and then fluorescence intensity is determined. Thereafter, a next cycle is performed by addition of a different selected amount of the targeted nuclease complex, followed again by determining fluorescence intensity. Multiple repeats of such cycles allow an amount of targeted nuclease complex at which optical cleavage kinetics are achieved to be determined.

The adapters comprising a primer binding site used in the methods of the invention may comprise a modified nucleic acid, a chemical moiety, an affinity moiety, or a fluorescent moiety. Such moiety is herein also referred to as a tag or label. Exemplary labels that can be detected in accordance with the invention, for example, when present on a solid surface include, but are not limited to, a chromophore; luminophore; fluorophore; optically encoded nanoparticles; particles encoded with a diffraction-grating; electrochemiluminescent label such as Ru(bpy)32+; or moiety that can be detected based on an optical characteristic. Fluorophores that are useful in the invention include, for example, fluorescent lanthanide complexes, including those of Europium and Terbium, fluorescein, rhodamine, tetramethylrhodamine, eosin, erythrosin, coumarin, methyl-coumarins, pyrene, Malacite green, Cy3, Cy5, stilbene, Lucifer Yellow, Cascade Blue™ Texas Red, alexa dyes, phycoerythin, bodipy, and others known in the art such as those described in Haugland, Molecular Probes Handbook, (Eugene, Oreg.) 6th Edition; The Synthegen catalog (Houston, Tex.), Lakowicz, Principles of Fluorescence Spectroscopy, 2nd Ed., Plenum Press New York (1999), or WO 98/59066, each of which is hereby incorporated by reference.

The optically detectable labels may be a particular size, shape, color, refractive index, or combination thereof. The optically detectable label should comprise a material and be of a size that can be resolvable using light spectroscopy, non-linear optical microscopy, phase contrast microscopy, fluorescence microscopy, including two-photon fluorescence microscopy, Raman spectroscopy, or a combination thereof. In certain example embodiments, the optically encoded particle may be naturally optically encoded, that is the particle is detectable using one of the above detection means without further modification. In certain other example embodiments, the particle material making up the optically detectable label is amenable to modification such that it can be made optically detectable using one of the above detection means, for example, by fluorescently or colorimetrically labeling the optically detectable label.

The optically detectable labels may comprise fluorophores, colloidal metal particles, nanoshells, nanotubes, nanorods, quantum dots, hydrogel particles, microspheres—such as polystyrene beads—liposomes, dendrimers, and metal-liposome particles. The optically detectable labels may be of any shape including, but not limited to, spherical, string-like, or rod-like. In certain example embodiments, the optically detectable labels are spherical in shape. In certain example embodiments, the optically detectable labels may be formed in a series of pre-defined shapes or sizes in order to distinguish the optically encoded particles by shape or size. In certain example embodiments, the optically detectable labels may have a diameter of approximately 50 nm to approximately 500 μm, or a length of approximately 50 nm to 500 μm.

In one example embodiment, the optically detectable label is a hydrogel particle. The hydrogel particle may be made from, for example, covalently cross-linked PEG with thiol-reactive functional groups, or low melting point agarose functionalized with streptavidin or nucleic acid. In certain example embodiments, the hydrogel particle may be approximately 50 nm to approximately 500 mm in size. In certain example embodiments, the hydrogel particle is fluorescently or colorimetrically labeled. In certain example embodiments, the optical label is incorporated within the hydrogel particle. In certain other example embodiments, the optical label is attached to the surface of the hydrogel particle.

In certain example embodiments, the optically detectable labels are quantum dots. In certain other example embodiments, the quantum dots may be incorporated into larger particles, such as those described above. The quantum dots may be made of semiconductor materials identifiable in the art as suitable for forming quantum dots. Exemplary quantum dots are available for purchase, e.g., from Sigma-Aldrich. The quantum dots may range in size from approximately 2 nm to approximately 20 nm.

In certain example embodiments, the optically detectable label is a colloidal metal particle. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A13+, Ru3+, Zn2+, Fe3+, Ni2+ and Ca2+ ions.

In certain example embodiments, the optically detectable particles are dendrimers. The dendrimer may be formed using standard methods known in the art. Exemplary dendrimers are available for purchase, e.g., from Sigma-Aldrich. The dendrimer may range in size from 5 nm to 500 nm, depending on the chosen size and length of, e.g., a central core, an interior dendritic structure (the branches), and an exterior surface with functional surface groups.

The term “unique molecular identifiers” (UMI) refers to a sequencing linker used in a method that uses molecular tags to detect and quantify unique amplified products. A UMI is used to distinguish effects through a single clone from multiple clones. A sequencer linker with a random sequence of between 4 and 20 base pairs is added to the 5′ end of the template, which is amplified and sequenced. Sequencing allows for high resolution reads, enabling accurate detection of true variants. As used herein, a “true variant” will be present in every amplified product originating from the original clone as identified by aligning all products with a UMI. Each clone amplified will have a different random UMI that will indicate that the amplified product originated from that clone. Background caused by the fidelity of the amplification process can be eliminated because true variants will be present in all amplified products and background representing random error will only be present in single amplification products (See e.g., Islam S. et al., 2014. Nature Methods No:11, 163-166). Not being bound by a theory, the UMI's are designed such that assignment to the original can take place despite up to 4-7 errors during amplification or sequencing. A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. One or more nucleic acid barcodes and/or UMIs can be attached, or “tagged,” to a target molecule and/or target nucleic acid, e.g. the immobilized nucleic acid molecules or adapters used in the methods of the invention. This attachment can be direct (for example, covalent or noncovalent binding of the barcode to the target molecule) or indirect (for example, via an additional molecule, for example, a specific binding agent, such as an antibody (or other protein) or a barcode receiving adaptor (or other nucleic acid molecule)). Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet location-, volume-, physical property-, or treatment condition-specific) nucleic acid barcodes. Thus, for example, each member of a set of nucleic acid barcodes, or other nucleic acid identifier or connector oligonucleotide, having identical or matched barcode sequences, may be associated with (for example, covalently bound to or a component of the same molecule as) a distinct or different UMI. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

Attachment of a barcode to target nucleic acid molecules can be performed using standard methods well known in the art. In certain embodiments, barcode tagging can occur via a barcode receiving adapter associated with (for example, attached to) a target nucleic acid molecule.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, a origin-specific barcode may be a nucleic acid including a barcode and a hybridization site for a specific primer. In particular embodiments, a set of origin-specific barcode includes a unique primer specific barcode made, for example, using a randomized oligo type NNNNNNNNNNNN.

Unique molecular identifiers are a subtype of nucleic acid barcode that can be used, for example, to normalize samples for variable amplification efficiency. For example, in various embodiments, featuring a solid or semisolid support (for example a hydrogel bead), to which nucleic acid barcodes (for example a plurality of barcode sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support or specific position on solid or semisolid support.

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

In some embodiments, the UMIs or barcodes are reversibly coupled to the solid support. In some embodiments, the barcodes further comprise a nucleic acid capture sequence that specifically binds to the target nucleic acid molecules. In specific embodiments, the barcodes include two or more populations of barcodes, wherein a first population comprises the nucleic acid target sequence and a second population comprises the specific binding agent that specifically binds to the target molecules. In some examples, the first population of barcodes further comprises a target nucleic acid barcode, wherein the target nucleic acid barcode identifies the population as one that labels nucleic acids. In some examples, the second population of barcodes further comprises a target molecule barcode, wherein the target molecule barcode identifies the population as one that labels target molecules.

In some embodiments, a barcode further includes a capture moiety, covalently or non-covalently linked. Thus, in some embodiments the barcode, and anything bound or attached thereto, that include a capture moiety are captured with a specific binding agent that specifically binds the capture moiety. In some embodiments, the capture moiety is adsorbed or otherwise captured on the solid surface. In specific embodiments, a targeting probe is labeled with biotin, for instance by incorporation of biotin-16-UTP during in vitro transcription, allowing later capture by streptavidin. Other means for labeling, capturing, and detecting an origin-specific barcode include: incorporation of aminoallyl-labeled nucleotides, incorporation of sulfhydryl-labeled nucleotides, incorporation of allyl- or azide-containing nucleotides, and many other methods described in Bioconjugate Techniques (2nd Ed), Greg T. Hermanson, Elsevier (2008), which is specifically incorporated herein by reference. In some embodiments, the targeting probes are covalently coupled to a solid support or other capture device prior to contacting the sample, using methods such as incorporation of aminoallyl-labeled nucleotides followed by 1-Ethyl-3-(3-dimethylaminopropyl)carbodiimide (EDC) coupling to a carboxy-activated solid support, or other methods described in Bioconjugate Techniques. In some embodiments the specific binding agent is has been immobilized for example on a solid support, thereby isolating the origin-specific barcode.

The methods of the invention typically use immobilized nucleic acid molecules wherein a nucleic acid modification, such as a strand break, is induced. Immobilization on the solid support typically is used to enable localization and identification of nucleic acid molecules. For instance, in certain embodiments the immobilized nucleic acid molecules may be sequenced both prior to and following contacting with an agent capable of inducing a nucleic acid modification. Localization and identification of each cluster of immobilized nucleic acid molecules allows direct comparison of the sequences obtained prior to and following said contacting of a single cluster of nucleic acid molecules. However, the methods of the invention can also be performed on nucleic acid molecules that are pre-registered in ways other than by immobilization on a solid support. As used herein “registered” in the context of nucleic acid molecules refers to the nucleic acid molecules comprising a characteristic that allows identification of single nucleic acid molecules or clusters comprising clones of single nucleic acid molecules. Preferably such methods allow specific sequencing of clusters of the registered nucleic acid molecules, both prior to and following contacting with an agent capable of inducing a nucleic acid modification in order to be able to compare both sequences obtained for a single cluster of nucleic acid molecules. For instance, registered nucleic acid molecules comprising a barcode can be sequenced using nanopore sequencing.

In nanopore sequencing, the pores (e.g. nanopores) may be solid state pores or non-solid-state pores (e.g. organic pores such as pores made from biological materials). The pores may have a functionality associated with them that facilitates detection of the sequence (e.g. may include enzymes or other materials such as polymerases attached near the pore to control the rate at which nucleotides flow through the pore, may include enzymes or other materials such as exonucleases which cleave off one or a few bases at a time, etc.). The pores may have a detection circuit associated with them (e.g. a patch clamp circuit, a tunneling electrode circuit, an optical sensor that detects labels on the fragments, etc.) that detects a sequence based on interaction of the fragment with the pore (e.g. passing the fragment through the pore, passing single nucleotides of the fragment through the pore, being peeled off by the pore, etc.).

In one aspect the invention therefore provides a method for detecting a nucleic acid modification, the method comprising:
i. sequencing at least part of one or more registered nucleic acid molecules;
ii. contacting said one or more registered nucleic acid molecules with an agent capable of inducing a nucleic acid modification; and
iii. sequencing at least part of said one or more registered nucleic acid molecules using a primer specifically binding to a primer binding site, said part comprising said nucleic acid modification,
iv. wherein said method comprises attaching an adapter comprising said primer binding site to said one or more registered nucleic acid molecules following said contacting step and prior to sequencing in step iii, or
v. wherein said one or more registered nucleic acid molecules that are contacted with said agent comprise an adapter comprising said primer binding site.

Said registered nucleic acid molecules preferably comprise a unique molecular identifier and/or barcode, more preferably a nucleic acid barcode. Methods and characteristics of immobilized nucleic acid molecules described herein can be equally applied to registered nucleic acid molecules. In other words, the registered nucleic acid molecules can be single stranded or double stranded DNA (e.g. gDNA or cDNA), or single stranded or double stranded RNA (e.g. mRNA). Similarly, modification induced in immobilized nucleic acid molecules can also be induced in registered, e.g. barcoded, nucleic acid molecules. Amplification and sequencing steps for registered, e.g. barcoded, nucleic acid molecules are described above and may include, but are not limited to, PCR, emulsion amplification and nanodrop sequencing.

The term “nuclease,” as used herein, refers to an agent capable of cleaving a phosphodiester bond connecting nucleotide residues in a nucleic acid molecule. In preferred embodiments, a nuclease is a protein, i.e. an enzyme that can bind a nucleic acid molecule and cleave a phosphodiester bond connecting nucleotide residues within the nucleic acid molecule. In preferred embodiments a nuclease is an endonuclease, cleaving a phosphodiester bonds within a polynucleotide chain. In preferred embodiments, a nuclease is a site-specific nuclease, cleaving a specific phosphodiester bond within a specific nucleotide sequence, which is also referred to herein as the “nuclease target site” or “nuclease target sequence”. In particular embodiments, a nuclease recognizes a single stranded target site. In other embodiments, a nuclease recognizes a double-stranded target site, such as a double-stranded DNA target site. Endonucleases may cut a double-stranded nucleic acid target site symmetrically resulting in ends comprising base-paired nucleotides, also referred to as blunt ends. Other endonucleases cuts a double-stranded nucleic acid target sites asymmetrically resulting in ends comprising unpaired nucleotides. Unpaired nucleotides at the end of a double-stranded DNA molecule are also referred to as “overhang”. In a preferred embodiment of the invention a doubled stranded break resulting in an overhang is blunt ended prior to adapter ligation and sequencing.

As used herein the term “targeted nuclease” refers to a nuclease, preferably an endonuclease, that acts on a specific target sequence in a nucleic acid sequence. In preferred embodiments a targeted nuclease is a guide RNA-directed nuclease. In preferred embodiments, the nuclease is part of a targeted nuclease complex. As used herein “target nuclease complex” refers to a complex comprising at least a targeted nuclease and optionally one further component. Said further component for instance is an agent capable of directing the nuclease to the target sequence. Such complex is for instance an RNA-nuclease complex, e.g. a guide RNA:nuclease complex.

Any nuclease able to induce a strand break in a nucleic acid molecule at a specific position and induce a single- or double-stranded break or a nick can be used as agent or nuclease in the present invention.

In certain embodiments, the agent capable of inducing a nucleic acid modification as described herein according to the invention is a (endo)nuclease or a variant thereof having altered or modified activity (i.e. a modified nuclease, as described herein elsewhere). In certain embodiments, said nuclease is a targeted or site-specific or homing nuclease or a variant thereof having altered or modified activity. In certain embodiments, said nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease. As used herein, the term “Cas” generally refers to a (modified) effector protein of the CRISPR/Cas system or complex, and can be without limitation a (modified) Cas9, Cpf1, C2c1, C2c2, C2c3, group 29, or group 30 protein. The term “derivative” as used herein in the context of a nuclease, e.g. a derivative of Cas9, Cpf1, C2c1, C2c2, C2c3, group 29 nuclease or group 30 nuclease refers to such modified nuclease, e.g. modified Cas9, Cpf1, C2c1, C2c2, C2c3, group 29 nuclease or group 30 nuclease. The term “Cas” may be used herein interchangeably with the terms “CRISPR” protein, “CRISPR/Cas protein”, “CRISPR effector”, “CRISPR/Cas effector”, “CRISPR enzyme”, “CRISPR/Cas enzyme” and the like, unless otherwise apparent, such as by specific and exclusive reference to Cas9. It is to be understood that the term “CRISPR protein” may be used interchangeably with “CRISPR enzyme”, irrespective of whether the CRISPR protein has altered, such as increased or decreased (or no) enzymatic activity, compared to the wild type CRISPR protein. Likewise, as used herein, in certain embodiments, where appropriate and which will be apparent to the skilled person, the term “nuclease” may refer to a modified nuclease wherein catalytic activity has been altered, such as having increased or decreased nuclease activity, or no nuclease activity at all, as well as nickase activity, as well as otherwise modified nuclease as defined herein elsewhere, unless otherwise apparent, such as by specific and exclusive reference to unmodified nuclease.

As used herein, the term “targeting” of a selected nucleic acid sequence means that a nuclease or nuclease complex is acting in a nucleotide sequence specific manner. For instance, in the context of the CRISPR/Cas system, the guide RNA is capable of hybridizing with a selected nucleic acid sequence. As uses herein, “hybridization” or “hybridizing” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PGR, or the cleavage of a polynucleotide by an enzyme. A sequence capable of hybridizing with a given sequence is referred to as the “complement” of the given sequence.

In certain embodiments, the nucleic acid modification is effected by a (modified) transcription activator-like effector nuclease (TALEN) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference. By means of further guidance, and without limitation, naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26. The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

In certain embodiments, the nucleic acid modification is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference. By means of further guidance, and without limitation, artificial zinc-finger (ZF) technology involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IIS restriction enzyme Fokl. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. With respect to general information on CRISPR/Cas Systems, components thereof, and delivery of such components, including methods, materials, delivery vehicles, vectors, particles, and making and using thereof, including as to amounts and formulations, as well as Cas9CRISPR/Cas-expressing eukaryotic cells, Cas-9 CRISPR/Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); 14/054,414 European Patent Applications EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), WO 2015/089351 (PCT/US2014/069897), WO 2015/089354 (PCT/US2014/069902), WO 2015/089364 (PCT/US2014/069925), WO 2015/089427 (PCT/US2014/070068), WO 2015/089462 (PCT/US2014/070127), WO 2015/089419 (PCT/US2014/070057), WO 2015/089465 (PCT/US2014/070135), WO 2015/089486 (PCT/US2014/070175), PCT/US2015/051691, PCT/US2015/051830. Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/835,973, 61/836,080, 61/836,101, and 61/836,127, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT/US2014/62558 filed Oct. 28, 2014, and U.S. Provisional Patent Applications Ser. Nos. 61/915,148, 61/915,150, 61/915,153, 61/915,203, 61/915,251, 61/915,301, 61/915,267, 61/915,260, and 61/915,397, each filed Dec. 12, 2013; 61/757,972 and 61/768,959, filed on Jan. 29, 2013 and Feb. 25, 2013; 62/010,888 and 62/010,879, both filed Jun. 11, 2014; 62/010,329, 62/010,439 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/055,484, 62/055,460 and 62/055,487, each filed Sep. 25, 2014; and 62/069,243, filed Oct. 27, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to PCT application designating, inter alia, the United States, application No. PCT/US14/41806, filed Jun. 10, 2014. [0002] Mention is also made of U.S. application 62/180,709, 17-Jun.-15, PROTECTED GUIDE RNAS (PGRNAS); U.S. application 62/091,455, filed, 12-Dec.-14, PROTECTED GUIDE RNAS (PGRNAS); U.S. application 62/096,708, 24-Dec.-14, PROTECTED GUIDE RNAS (PGRNAS); U.S. applications 62/091,462, 12-Dec.-14, 62/096,324, 23-Dec.-14, 62/180,681, 17 Jun. 2015, and 62/237,496, 5 Oct. 2015, DEAD GUIDES FOR CRISPR TRANSCRIPTION FACTORS; U.S. application 62/091,456, 12-Dec.-14 and 62/180,692, 17 Jun. 2015, ESCORTED AND FUNCTIONALIZED GUIDES FOR CRISPR-CAS SYSTEMS; U.S. application 62/091,461, 12-Dec.-14, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19-Dec.-14, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24-Dec.-14, ENGINEERING OF SYSTEMS, METHODS AND OPTIMIZED ENZYME AND GUIDE SCAFFOLDS FOR SEQUENCE MANIPULATION; U.S. application 62/098,059, 30-Dec.-14, 62/181,641, 18 Jun. 2015, and 62/181,667, 18 Jun. 2015, RNA-TARGETING SYSTEM; U.S. application 62/096,656, 24-Dec.-14 and 62/181,151, 17 Jun. 2015, CRISPR HAVING OR ASSOCIATED WITH DESTABILIZATION DOMAINS; U.S. application 62/096,697, 24-Dec.-14, CRISPR HAVING OR ASSOCIATED WITH AAV; U.S. application 62/098,158, 30-Dec.-14, ENGINEERED CRISPR COMPLEX INSERTIONAL TARGETING SYSTEMS; U.S. application 62/151,052, 22-Apr.-15, CELLULAR TARGETING FOR EXTRACELLULAR EXOSOMAL REPORTING; U.S. application 62/054,490, 24-Sep.-14, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12-Feb.-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25-Sep.-14, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4-Dec.-14, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24-Sep.-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23-Oct.-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24-Sep.-14 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24-Sep.-14, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25-Sep.-14, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25-Sep.-14, MULTIFUNCTIONAL-CRISPR COMPLEXES AND/OR OPTIMIZED ENZYME LINKED FUNCTIONAL-CRISPR COMPLEXES; U.S. application 62/087,475, 4-Dec.-14 and 62/181,690, 18 Jun. 2015, FUNCTIONAL SCREENING WITH OPTIMIZED FUNCTIONAL CRISPR-CAS SYSTEMS; U.S. application 62/055,487, 25-Sep.-14, FUNCTIONAL SCREENING WITH OPTIMIZED FUNCTIONAL CRISPR-CAS SYSTEMS; U.S. application 62/087,546, 4-Dec.-14 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30-Dec.-14, CRISPR MEDIATED IN VIVO MODELING AND GENETIC SCREENING OF TUMOR GROWTH AND METASTASIS. Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, and Attorney Docket No. 46783.01.2128, filed 22-Oct.-2015, NOVEL CRISPR ENZYMES AND SYSTEMS, U.S. application 62/232,067, 24-Sep.-2015, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES. European patent application EP3009511. Reference is further made to Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science Feb. 15; 339(6121):819-23 (2013); RNA-guided editing of bacterial genomes using CRISPR-Cas systems. Jiang W., Bikard D., Cox D., Zhang F, Marraffini L A. Nat Biotechnol Mar.; 31(3):233-9 (2013); One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013); Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. 2013 Aug. 22; 500(7463):472-6. doi: 10.1038/Nature12466. Epub 2013 Aug. 23; Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell Aug. 28. pii: S0092-8674 (13)01015-5. (2013); DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013); Genome engineering using the CRISPR-Cas9 system. Ran, F A., Hsu, P D., Wright, J., Agarwala, V., Scott, D A., Zhang, F. Nature Protocols Nov.; 8(11):2281-308. (2013); Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science Dec. 12. (2013). [Epub ahead of print]; Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell Feb. 27. (2014). 156(5):935-49; Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. (2014) Apr. 20. doi: 10.1038/nbt.2889; CRISPR-Cas9 Knockin Mice for Genome Editing and Cancer Modeling, Platt et al., Cell 159(2): 440-455 (2014) DOI: 10.1016/j.cell.2014.Sep.014; Development and Applications of CRISPR-Cas9 for Genome Engineering, Hsu et al, Cell 157, 1262-1278 (Jun. 5, 2014) (Hsu 2014); Genetic screens in human cells using the CRISPR/Cas9 system, Wang et al., Science. 2014 Jan. 3; 343(6166): 80-84. doi:10.1126/science.1246981; Rational design of highly active sgRNAs for CRISPR-Cas9-mediated gene inactivation, Doench et al., Nature Biotechnology 32(12):1262-7 (2014) published online 3 Sep. 2014; doi:10.1038/nbt.3026, and In vivo interrogation of gene function in the mammalian brain using CRISPR-Cas9, Swiech et al, Nature Biotechnology 33, 102-106 (2015) published online 19 Oct. 2014; doi:10.1038/nbt.3055, Cpf1 Is a Single RNA-Guided Endonuclease of a Class 2 CRISPR-Cas System, Zetsche et al., Cell 163, 1-13 (2015); Discovery and Functional Characterization of Diverse Class 2 CRISPR-Cas Systems, Shmakov et al., Mol Cell 60(3): 385-397 (2015); C2c2 is a single-component programmable RNA-guided RNA-targeting CRISPR effector, Abudayyeh et al, Science (2016) published online Jun. 2, 2016 doi: 10.1126/science.aaf5573. Each of these publications, patents, patent publications, and applications, and all documents cited therein or during their prosecution (“appin cited documents”) and all documents cited or referenced in the appin cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appin cited documents) are incorporated herein by reference to the same extent as if each individual document was specifically and individually indicated to be incorporated by reference.

Preferred agents in the context of this invention comprise a CRISPR/Cas system or complex. In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cpf1).

As used herein, the term “crRNA” or “guide RNA” or “single guide RNA” or “sgRNA” or “one or more nucleic acid components” of a CRISPR/Cas locus effector protein, as applicable, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay.

A guide sequence, and hence a nucleic acid-targeting guide RNA may be selected to target any target nucleic acid sequence. The target sequence may be DNA. The target sequence may be genomic DNA. The target sequence may be mitochondrial DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In certain embodiments, the gRNA comprises a stem loop, preferably a single stem loop. In certain embodiments, the direct repeat sequence forms a stem loop, preferably a single stem loop. In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In particular embodiments, the CRISPR/Cas system requires a tracrRNA. The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and gRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop may correspond to the tracr mate sequence, and the portion of the sequence 3′ of the loop then corresponds to the tracr sequence. In a hairpin structure the portion of the sequence 5′ of the final “N” and upstream of the loop may alternatively correspond to the tracr sequence, and the portion of the sequence 3′ of the loop corresponds to the tracr mate sequence. In alternative embodiments, the CRISPR/Cas system does not require a tracrRNA, as is known by the skilled person.

In certain embodiments, the guide RNA (capable of guiding Cas to a target locus) may comprise (1) a guide sequence capable of hybridizing to a target locus and (2) a tracr mate or direct repeat sequence (in 5′ to 3′ orientation, or alternatively in 3′ to 5′ orientation, depending on the type of Cas protein, as is known by the skilled person). In particular embodiments, the CRISPR/Cas protein is characterized in that it makes use of a guide RNA comprising a guide sequence capable of hybridizing to a target locus and a direct repeat sequence, and does not require a tracrRNA. In particular embodiments, where the CRISPR/Cas protein is characterized in that it makes use of a tracrRNA, the guide sequence, tracr mate, and tracr sequence may reside in a single RNA, i.e. an sgRNA (arranged in a 5′ to 3′ orientation or alternatively arranged in a 3′ to 5′ orientation), or the tracr RNA may be a different RNA than the RNA containing the guide and tracr mate sequence. In these embodiments, the tracr hybridizes to the tracr mate sequence and directs the CRISPR/Cas complex to the target sequence.

Typically, in the context of an endogenous nucleic acid-targeting system, formation of a nucleic acid-targeting complex (comprising a guide RNA hybridized to a target sequence and complexed with one or more nucleic acid-targeting effector proteins) results in modification (such as cleavage) of one or both DNA or RNA strands in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest). The skilled person will be aware of specific cut sites for selected CRISPR/Cas systems, relative to the target sequence, which as is known in the art may be within the target sequence or alternatively 3′ or 5′ of the target sequence.

In some embodiments, the unmodified nucleic acid-targeting effector protein may have nucleic acid cleavage activity. In some embodiments, the nuclease as described herein may direct cleavage of one or both nucleic acid (DNA, RNA, or hybrids, which may be single or double stranded) strands at the location of or near a target sequence, such as within the target sequence and/or within the complement of the target sequence or at sequences associated with the target sequence. In some embodiments, the nucleic acid-targeting effector protein may direct cleavage of one or both DNA or RNA strands within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt (e.g. for Cas9, such as SaCas9 or SpCas9). In some embodiments, the cleavage may be staggered (e.g. for Cpf1), i.e. generating sticky ends. In some embodiments, the cleavage is a staggered cut with a 5′ overhang. In some embodiments, the cleavage is a staggered cut with a 5′ overhang of 1 to 5 nucleotides, preferably of 4 or 5 nucleotides. In some embodiments, the cleavage site is upstream of the PAM. In some embodiments, the cleavage site is downstream of the PAM. In some embodiments, the nucleic acid-targeting effector protein that may be mutated with respect to a corresponding wild-type enzyme such that the mutated nucleic acid-targeting effector protein lacks the ability to cleave one or both DNA or RNA strands of a target polynucleotide containing a target sequence. As a further example, two or more catalytic domains of a Cas protein (e.g. RuvC I, RuvC II, and RuvC III or the HNH domain of a Cas9 protein) may be mutated to produce a mutated Cas protein substantially lacking all DNA cleavage activity. In some embodiments, a nucleic acid-targeting effector protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. As used herein, the term “modified” Cas generally refers to a Cas protein having one or more modifications or mutations (including point mutations, truncations, insertions, deletions, chimeras, fusion proteins, etc.) compared to the wild type Cas protein from which it is derived. By derived is meant that the derived enzyme is largely based, in the sense of having a high degree of sequence homology with, a wildtype enzyme, but that it has been mutated (modified) in some way as known in the art or as described herein.

In certain embodiments, the target sequence should be associated with a PAM (protospacer adjacent motif) or PFS (protospacer flanking sequence or site); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). Examples of PAM sequences are given in the examples section below, and the skilled person will be able to identify further PAM sequences for use with a given CRISPR enzyme. Further, engineering of the PAM Interacting (PI) domain may allow programming of PAM specificity, improve target site recognition fidelity, and increase the versatility of the Cas, e.g. Cas9, genome engineering platform. Cas proteins, such as Cas9 proteins may be engineered to alter their PAM specificity, for example as described in Kleinstiver BP et al. Engineered CRISPR-Cas9 nucleases with altered PAM specificities. Nature. 2015 Jul. 23; 523(7561):481-5. doi: 10.1038/nature14592. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence. The skilled person will understand that other Cas proteins may be modified analogously.

The Cas protein as referred to herein, such as without limitation Cas9, Cpf1, C2c1, C2c2, C2c3, group 29, or group 30 protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is (modified) Cpf1, preferably Acidaminococcus sp., such as Acidaminococcus sp. BV3L6 Cpf1 (AsCpf1) or Lachnospiraceae bacterium Cpf1, such as Lachnospiraceae bacterium MA2020 or Lachnospiraceae bacterium MD2006 (LbCpf1). In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is group 29 or group 30 protein.

In certain embodiments, the nuclease as referred to herein is modified. As used herein, the term “modified” refers to a nuclease which may or may not have an altered functionality. By means of example, and in particular with reference to Cas proteins, modifications which do not result in an altered functionality include for instance codon optimization for expression into a particular host, or providing the nuclease with a particular marker (e.g. for visualization). Modifications with may result in altered functionality may also include mutations, including point mutations, insertions, deletions, truncations (including split nucleases), etc., as well as chimeric nucleases (e.g. comprising domains from different orthologues or homologues) or fusion proteins. Fusion proteins may without limitation include for instance fusions with heterologous domains or functional domains (e.g. localization signals, catalytic domains, etc.). Accordingly, in certain embodiments, the modified nuclease may be used as a generic nucleic acid binding protein with fusion to or being operably linked to a functional domain. In certain embodiments, various different modifications may be combined (e.g. a mutated nuclease which is catalytically inactive and which further is fused to a functional domain, such as for instance to induce DNA methylation or another nucleic acid modification, such as including without limitation a break (e.g. by a different nuclease (domain)), a mutation, a deletion, an insertion, a replacement, a ligation, a digestion, a break or a recombination). As used herein, “altered functionality” includes without limitation an altered specificity (e.g. altered target recognition, increased (e.g. “enhanced” Cas proteins) or decreased specificity, or altered PAM recognition), altered activity (e.g. increased or decreased catalytic activity, including catalytically inactive nucleases or nickases), and/or altered stability (e.g. fusions with destalilization domains). Suitable heterologous domains include without limitation a nuclease, a ligase, a repair protein, a methyltransferase, (viral) integrase, a recombinase, a transposase, an argonaute, a cytidine deaminase, a retron, a group II intron, a phosphatase, a phosphorylase, a sulpfurylase, a kinase, a polymerase, an exonuclease, etc. Examples of all these modifications are known in the art. It will be understood that a “modified” nuclease as referred to herein, and in particular a “modified” Cas or “modified” CRISPR/Cas system or complex preferably still has the capacity to interact with or bind to the polynucleic acid (e.g. in complex with the gRNA).

By means of further guidance and without limitation, in certain embodiments, the nuclease may be modified as detailed below. As already indicated, more than one of the indicated modifications may be combined. For instance, codon optimization may be combined with NLS or NES fusions, catalytically inactive nuclease modifications or nickase mutants may be combined with fusions to functional (heterologous) domains, etc.

In certain embodiments, the nuclease, and in particular the Cas proteins of prokaryotic origin, may be codon optimized for expression into a particular host (cell). An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a Cas is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, P A), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid. Codon optimization may be for expression into any desired host (cell), including mammalian, plant, algae, or yeast.

In certain embodiments, the nuclease, in particular the Cas protein, may comprise one or more modifications resulting in enhanced activity and/or specificity, such as including mutating residues that stabilize the targeted or non-targeted strand (e.g. eCas9; “Rationally engineered Cas9 nucleases with improved specificity”, Slaymaker et al. (2016), Science, 351(6268):84-88, incorporated herewith in its entirety by reference). In certain embodiments, the altered or modified activity of the engineered CRISPR protein comprises increased targeting efficiency or decreased off-target binding. In certain embodiments, the altered activity of the engineered CRISPR protein comprises modified cleavage activity. In certain embodiments, the altered activity comprises increased cleavage activity as to the target polynucleotide loci. In certain embodiments, the altered activity comprises decreased cleavage activity as to the target polynucleotide loci. In certain embodiments, the altered activity comprises decreased cleavage activity as to off-target polynucleotide loci. In certain embodiments, the altered or modified activity of the modified nuclease comprises altered helicase kinetics. In certain embodiments, the modified nuclease comprises a modification that alters association of the protein with the nucleic acid molecule comprising RNA (in the case of a Cas protein), or a strand of the target polynucleotide loci, or a strand of off-target polynucleotide loci. In an aspect of the invention, the engineered CRISPR protein comprises a modification that alters formation of the CRISPR complex. In certain embodiments, the altered activity comprises increased cleavage activity as to off-target polynucleotide loci. Accordingly, in certain embodiments, there is increased specificity for target polynucleotide loci as compared to off-target polynucleotide loci. In other embodiments, there is reduced specificity for target polynucleotide loci as compared to off-target polynucleotide loci. In certain embodiments, the mutations result in decreased off-target effects (e.g. cleavage or binding properties, activity, or kinetics), such as in case for Cas proteins for instance resulting in a lower tolerance for mismatches between target and gRNA. Other mutations may lead to increased off-target effects (e.g. cleavage or binding properties, activity, or kinetics). Other mutations may lead to increased or decreased on-target effects (e.g. cleavage or binding properties, activity, or kinetics). In certain embodiments, the mutations result in altered (e.g. increased or decreased) helicase activity, association or formation of the functional nuclease complex (e.g. CRISPR/Cas complex). In certain embodiments, the mutations result in an altered PAM recognition, i.e. a different PAM may be (in addition or in the alternative) be recognized, compared to the unmodified Cas protein (see e.g. “Engineered CRISPR-Cas9 nucleases with altered PAM specificities”, Kleinstiver et al. (2015), Nature, 523(7561):481-485, incorporated herein by reference in its entirety). Particularly preferred mutations include positively charged residues and/or (evolutionary) conserved residues, such as conserved positively charged residues, in order to enhance specificity. In certain embodiments, such residues may be mutated to uncharged residues, such as alanine. By means of example, and without limitation, SpCas9 may be mutated as described above in a RuvCI, RuvCIII, RuvCIII or HNH domain. In certain of the above-described non-naturally-occurring CRISPR enzymes, the enzyme is modified by mutation of one or more residues including but not limited positions 12, 13, 63, 415, 610, 775, 779, 780, 810, 832, 848, 855, 861, 862, 866, 961, 968, 974, 976, 982, 983, 1000, 1003, 1014, 1047, 1060, 1107, 1108, 1109, 1114, 1129, 1240, 1289, 1296, 1297, 1300, 1311, and 1325 with reference to amino acid position numbering of SpCas9. In certain of the above-described non-naturally-occurring CRISPR enzymes, the enzyme is modified by mutation and comprises one or more alanine substitutions at residues including but not limited positions 63, 415, 775, 779, 780, 810, 832, 848, 855, 861, 862, 866, 961, 968, 974, 976, 982, 983, 1000, 1003, 1014, 1047, 1060, 1107, 1108, 1109, 1114, 1129, 1240, 1289, 1296, 1297, 1300, 1311, or 1325 with reference to amino acid position numbering of SpCas9. In certain of the above-described non-naturally-occurring CRISPR enzymes, the enzyme is modified by mutation and comprises one or more substitions of K775A, E779L, Q807A, R780A, K810A, R832A, K848A, K855A, K862A, K866A, K961A, K968A, K974A, R976A, H982A, H983A, K1000A, K1014A, K1047A, K1060A, K1003A, K1107A, S1109A, H1240A, K1289A, K1296A, H1297A, K1300A, H1311A, or K1325A. In certain of the above-described non-naturally-occurring CRISPR enzymes, the enzyme is modified by mutation and comprises two or more substitutions, wherein the two or more substitutions include without limitation R783A and A1322T, or R780A and K810A, or ER780A and K855A, or R780A and R976A, or K848A and R976A, or K855A and R976A, and R780A and K848A, or K810A and K848A, or K848A and K855A, or K810A and K855A, or H982A and R1060A, or H982A and R1003A, or K1003A and R1060A, or R780A and H982A, or K810A and H982A, or K848A and H982A, or K855A and H982A, or R780A and K1003A, or K810A and R1003A, or K848A and K1003A, or K848A and K1007A, or R780A and R1060A, or K810A and R1060A, or K848A and R1060A, or R780A and R1114A, or K848A and R1114A, or R63A and K855A, or R63A and H982A, or H415A and R780A, or H415A and K848A, or K848A and E1108A, or K810A and K1003A, or R780A and R1060A, K810A and R1060A, or K848A and R1060A. In certain of the above-described non-naturally-occurring CRISPR enzymes, the enzyme is modified by mutation and comprises three or more substitutions, wherein the three or more substitutions include without limitation H982A, K1003A, and K1129E, or R780A, K1003A, and R1060A, or K810A, K1003A, and R1060A, or K848A, K1003A, and R1060A, or K855A, K1003A, and R1060A, or H982A, K1003A, and R1060A, or R63A, K848A, and R1060A, or T131, R63A, and K810A, or G12D, R63A, and R1060A. In certain of the above-described non-naturally-occurring CRISPR enzymes, the enzyme is modified by mutation and comprises four or more substitutions, wherein the four or more substitutions include without limitation R63A, E610G, K855A, and R1060A, or R63A, K855A, R1060A, and E610G. For example, one or more of (positively charged) residues R63 to K1325 or K775 to K1325 of Streptococcus pyogenes Cas9 (SpCas9), such as SpCas9 K855A, SpCas9 K810A/K1003A/R1060A, and SpCas9 K848A/K1003A/R1060A, or a corresponding region in another Cas9 ortholog may be mutated, or one or more of (positively charged) residues K37 to K736 of Staphylococcus aureus Cas9 (SaCas9) or a corresponding region in another Cas9 ortholog may be mutated. In general, in certain embodiments, and exemplified for Cas9, the mutations described provide for enhancing conformational rearrangement of Cas9 domains to positions that results in cleavage at on-target sits and avoidance of those conformational states at off-target sites. Cas9 cleaves target DNA in a series of coordinated steps. First, the PAM-interacting domain recognizes the PAM sequence 5′ of the target DNA. After PAM binding, the first 10-12 nucleotides of the target sequence (seed sequence) are sampled for sgRNA:DNA complementarity, a process dependent on DNA duplex separation. If the seed sequence nucleotides complement the sgRNA, the remainder of DNA is unwound and the full length of sgRNA hybridizes with the target DNA strand. The nt-groove between the RuvC and HNH domains stabilizes the non-targeted DNA strand and facilitates unwinding through non-specific interactions with positive charges of the DNA phosphate backbone. RNA:cDNA and Cas9:ncDNA interactions drive DNA unwinding in competition against cDNA:ncDNA rehybridization. Other cas9 domains affect the conformation of nuclease domains as well, for example linkers connecting HNH with RuvCII and RuvCIII. Accordingly, the mutations provided encompass, without limitation, RuvCI, RuvCIII, RuvCIII and HNH domains and linkers. Conformational changes in Cas9 brought about by target DNA binding, including seed sequence interaction, and interactions with the target and non-target DNA strand determine whether the domains are positioned to trigger nuclease activity. Thus, the mutations provided herein demonstrate and enable modifications that go beyond PAM recognition and RNA-DNA base pairing. Suitable residues to mutate may advantageously be identified based on for instance the crystal structure of the nuclease.

In certain embodiments, the nuclease, in particular the Cas protein, may comprise one or more modifications resulting in a nuclease that has reduced or no catalytic activity, or alternatively (in case of nucleases that target double stranded nucleic acids) resulting in a nuclease that only cleaves one strand, i.e. a nickase. By means of further guidance, and without limitation, for example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As further guidance, where the enzyme is not SpCas9, mutations may be made at any or all residues corresponding to positions 10, 762, 840, 854, 863 and/or 986 of SpCas9 (which may be ascertained for instance by standard sequence comparison tools). In particular, any or all of the following mutations are preferred in SpCas9: D10A, E762A, H840A, N854A, N863A and/or D986A; as well as conservative substitution for any of the replacement amino acids is also envisaged. As a further example, two or more catalytic domains of Cas9 (RuvC I, RuvC II, and RuvC III or the HNH domain) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a Cas is considered to substantially lack all DNA cleavage activity when the DNA cleavage activity of the mutated enzyme is about no more than 25%, 10%, 5%, 1%, 0.1%, 0.01%, or less of the DNA cleavage activity of the non-mutated form of the enzyme; an example can be when the DNA cleavage activity of the mutated form is nil or negligible as compared with the non-mutated form. Thus, the Cas may comprise one or more mutations and may be used as a generic DNA binding protein with or without fusion to a functional domain. The mutations may be artificially introduced mutations or gain- or loss-of-function mutations. The mutations may include but are not limited to mutations in one of the catalytic domains (e.g., D10 and H840) in the RuvC and HNH catalytic domains respectively; or the CRISPR enzyme can comprise one or more mutations selected from the group consisting of D10A, E762A, H840A, N854A, N863A or D986A and/or one or more mutations in a RuvC1 or HNH domain of the Cas or has a mutation as otherwise as discussed herein.

In certain embodiments, the nuclease is a split nuclease (see e.g. “A split-Cas9 architecture for inducible genome editing and transcription modulation”, Zetsche et al. (2015), Nat Biotechnol. 33(2):139-42, incorporated herein by reference in its entirety). In a split nuclease, the activity (which may be a modified activity, as described herein elsewhere), relies on the two halves of the split nuclease to be joined, i.e. each half of the split nuclease does not possess the required activity, until joined. As further guidance, and without limitation, with specific reference to Cas9, a split Cas9 may result from splitting the Cas9 at any one of the following split points, according or with reference to SpCas9: a split position between 202A/203S; a split position between 255F/256D; a split position between 310E/3111; a split position between 534R/535K; a split position between 572E/573C; a split position between 713S/714G; a split position between 1003L/104E; a split position between 1054G/1055E; a split position between 1114N/1115S; a split position between 1152K/1153S; a split position between 1245K/1246G; or a split between 1098 and 1099. Identifying potential split sides is most simply done with the help of a crystal structure. For Sp mutants, it should be readily apparent what the corresponding position for, for example, a sequence alignment. For non-Sp enzymes one can use the crystal structure of an ortholog if a relatively high degree of homology exists between the ortholog and the intended Cas9. Ideally, the split position should be located within a region or loop. Preferably, the split position occurs where an interruption of the amino acid sequence does not result in the partial or full destruction of a structural feature (e.g. alpha-helixes or beta-sheets). Unstructured regions (regions that did not show up in the crystal structure because these regions are not structured enough to be “frozen” in a crystal) are often preferred options. In certain embodiments, a functional domain may be provided on each of the split halves, thereby allowing the formation of homodimers or heterodimers. The functional domains may be (inducible) interact, thereby joining the split halves, and reconstituting (modified) nuclease activity. By means of example, an inducer energy source may inducibly allow dimerization of the split halves, through appropriate fusion partners. An inducer energy source may be considered to be simply an inducer or a dimerizing agent. The term ‘inducer energy source’ is used herein throughout for consistency. The inducer energy source (or inducer) acts to reconstitute the Cas9. In some embodiments, the inducer energy source brings the two parts of the Cas9 together through the action of the two halves of the inducible dimer. The two halves of the inducible dimer therefore are brought tougher in the presence of the inducer energy source. The two halves of the dimer will not form into the dimer (dimerize) without the inducer energy source. Thus, the two halves of the inducible dimer cooperate with the inducer energy source to dimerize the dimer. This in turn reconstitutes the Cas9 by bringing the first and second parts of the Cas9 together. The CRISPR enzyme fusion constructs each comprise one part of the split Cas9. These are fused, preferably via a linker such as a GlySer linker described herein, to one of the two halves of the dimer. The two halves of the dimer may be substantially the same two monomers that together that form the homodimer, or they may be different monomers that together form the heterodimer. As such, the two monomers can be thought of as one half of the full dimer. The Cas9 is split in the sense that the two parts of the Cas9 enzyme substantially comprise a functioning Cas9. That Cas9 may function as a genome editing enzyme (when forming a complex with the target DNA and the guide), such as a nickase or a nuclease (cleaving both strands of the DNA), or it may be a deadCas9 which is essentially a DNA-binding protein with very little or no catalytic activity, due to typically two or more mutations in its catalytic domains as described herein further.

In certain embodiments, the nuclease may comprise one or more additional (heterologous) functional domains, i.e. the modified nuclease is a fusion protein comprising the nuclease itself and one or more additional domains, which may be fused C-terminally or N-terminally to the nuclease, or alternatively inserted at suitable and appropriate sited internally within the nuclease (preferably without perturbing its function, which may be an otherwise modified function, such as including reduced or absent catalytic activity, nickase activity, etc.). any type of functional domain may suitably be used, such as without limitation including functional domains having one or more of the following activities: (DNA or RNA) methyltransferase activity, methylase activity, demethylase activity, DNA hydroxylmethylase domain, histone acetylase domain, histone deacetylases domain, transcription or translation activation activity, transcription or translation repression activity, transcription or translation release factor activity, histone modification activity, nuclease activity, single-strand RNA cleavage activity, double-strand RNA cleavage activity, single-strand DNA cleavage activity, double-strand DNA cleavage activity, nucleic acid binding activity, a protein acetyltransferase, a protein deacetylase, a protein methyltransferase, a protein deaminase, a protein kinase, a protein phosphatase, transposase domain, integrase domain, recombinase domain, resolvase domain, invertase domain, protease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase, histone tail protease, HDACs, histone methyltransferases (HMTs), and histone acetyltransferase (HAT) inhibitors, as well as HDAC and HMT recruiting proteins, HDAC Effector Domains, HDAC Recruiter Effector Domains, Histone Methyltransferase (HMT) Effector Domains, Histone Methyltransferase (HMT) Recruiter Effector Domains, or Histone Acetyltransferase Inhibitor Effector Domains. In some embodiments, the functional domain is an epigenetic regulator; see, e.g., Zhang et al., U.S. Pat. No. 8,507,272 (incorporated herein by reference in its entirety). In some embodiments, the functional domain is a transcriptional activation domain, such as VP64, p65, MyoD1, HSF1, RTA, SETT/9 or a histone acetyltransferase. In some embodiments, the functional domain is a transcription repression domain, such as KRAB. In some embodiments, the transcription repression domain is SID, or concatemers of SID (eg SID4X), NuE, or NcoR. In some embodiments, the functional domain is an epigenetic modifying domain, such that an epigenetic modifying enzyme is provided. In some embodiments, the functional domain is an activation domain, which may be the P65 activation domain. In some embodiments, the functional domain comprises nuclease activity. In one such embodiment, the functional domain may comprise Fokl. Mention is made of U.S. Pat. Pub. 2014/0356959, U.S. Pat. Pub. 2014/0342456, U.S. Pat. Pub. 2015/0031132, and Mali, P. et al., 2013, Science 339(6121):823-6, doi: 10.1126/science.1232033, published online 3 Jan. 2013 and through the teachings herein the invention comprehends methods and materials of these documents applied in conjunction with the teachings herein. It is to be understood that also destabilization domains or localization domains as described herein elsewhere are encompassed by the generic term “functional domain”. In certain embodiments, one or more functional domains are associated with the nuclease itself. In some embodiments, one or more functional domains are associated with an adaptor protein, for example as used with the modified guides of Konnerman et al. (Nature 517(7536): 583-588, 2015; incorporated herein by reference in its entirety), and hene form part of a Synergistic activator mediator (SAM) complex. The adaptor proteins may include but are not limited to orthogonal RNA-binding protein/aptamer combinations that exist within the diversity of bacteriophage coat proteins. A list of such coat proteins includes, but is not limited to: Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7 s and PRR1. These adaptor proteins or orthogonal RNA binding proteins can further recruit effector proteins or fusions which comprise one or more functional domains.

In certain embodiments, the nuclease, in particular the Cas protein, may comprise one or more modifications resulting in a destabilized nuclease when expressed in a host (cell). Such may be achieved by fusion of the nuclease with a destabilization domain (DD). Destabilizing domains have general utility to confer instability to a wide range of proteins; see, e.g., Miyazaki, J Am Chem Soc. Mar. 7, 2012; 134(9): 3942-3945, incorporated herein by reference. CMP8 or 4-hydroxytamoxifen can be destabilizing domains. More generally, A temperature-sensitive mutant of mammalian DHFR (DHFRts), a destabilizing residue by the N-end rule, was found to be stable at a permissive temperature but unstable at 37° C. The addition of methotrexate, a high-affinity ligand for mammalian DHFR, to cells expressing DHFRts inhibited degradation of the protein partially. This was an important demonstration that a small molecule ligand can stabilize a protein otherwise targeted for degradation in cells. A rapamycin derivative was used to stabilize an unstable mutant of the FRB domain of mTOR (FRB*) and restore the function of the fused kinase, GSK-3β.6,7 This system demonstrated that ligand-dependent stability represented an attractive strategy to regulate the function of a specific protein in a complex biological environment. A system to control protein activity can involve the DD becoming functional when the ubiquitin complementation occurs by rapamycin induced dimerization of FK506-binding protein and FKBP12. Mutants of human FKBP12 or ecDHFR protein can be engineered to be metabolically unstable in the absence of their high-affinity ligands, Shield-1 or trimethoprim (TMP), respectively. These mutants are some of the possible destabilizing domains (DDs) useful in the practice of the invention and instability of a DD as a fusion with a CRISPR enzyme confers to the CRISPR protein degradation of the entire fusion protein by the proteasome. Shield-1 and TMP bind to and stabilize the DD in a dose-dependent manner. The estrogen receptor ligand binding domain (ERLBD, residues 305-549 of ERS1) can also be engineered as a destabilizing domain. Since the estrogen receptor signaling pathway is involved in a variety of diseases such as breast cancer, the pathway has been widely studied and numerous agonist and antagonists of estrogen receptor have been developed. Thus, compatible pairs of ERLBD and drugs are known. There are ligands that bind to mutant but not wild-type forms of the ERLBD. By using one of these mutant domains encoding three mutations (L384M, M421G, G521R)12, it is possible to regulate the stability of an ERLBD-derived DD using a ligand that does not perturb endogenous estrogen-sensitive networks. An additional mutation (Y537S) can be introduced to further destabilize the ERLBD and to configure it as a potential DD candidate. This tetra-mutant is an advantageous DD development. The mutant ERLBD can be fused to a CRISPR enzyme and its stability can be regulated or perturbed using a ligand, whereby the CRISPR enzyme has a DD. Another DD can be a 12-kDa (107-amino-acid) tag based on a mutated FKBP protein, stabilized by Shield1 ligand; see, e.g., Nature Methods 5, (2008). For instance a DD can be a modified FK506 binding protein 12 (FKBP12) that binds to and is reversibly stabilized by a synthetic, biologically inert small molecule, Shield-1; see, e.g., Banaszynski L A, Chen L C, Maynard-Smith L A, Ooi A G, Wandless T J. A rapid, reversible, and tunable method to regulate protein function in living cells using synthetic small molecules. Cell. 2006; 126:995-1004; Banaszynski L A, Sellmyer M A, Contag C H, Wandless T J, Thorne S H. Chemical control of protein stability and function in living mice. Nat Med. 2008; 14:1123-1127; Maynard-Smith L A, Chen L C, Banaszynski L A, Ooi A G, Wandless T J. A directed approach for engineering conditional protein stability using biologically silent small molecules. The Journal of biological chemistry. 2007; 282:24866-24872; and Rodriguez, Chem Biol. Mar. 23, 2012; 19(3): 391-398—all of which are incorporated herein by reference and may be employed in the practice of the invention in selected a DD to associate with a CRISPR enzyme in the practice of this invention. As can be seen, the knowledge in the art includes a number of DDs, and the DD can be associated with, e.g., fused to, advantageously with a linker, to a CRISPR enzyme, whereby the DD can be stabilized in the presence of a ligand and when there is the absence thereof the DD can become destabilized, whereby the CRISPR enzyme is entirely destabilized, or the DD can be stabilized in the absence of a ligand and when the ligand is present the DD can become destabilized; the DD allows the CRISPR enzyme and hence the CRISPR-Cas complex or system to be regulated or controlled—turned on or off so to speak, to thereby provide means for regulation or control of the system, e.g., in an in vivo or in vitro environment. For instance, when a protein of interest is expressed as a fusion with the DD tag, it is destabilized and rapidly degraded in the cell, e.g., by proteasomes. Thus, absence of stabilizing ligand leads to a D associated Cas being degraded. When a new DD is fused to a protein of interest, its instability is conferred to the protein of interest, resulting in the rapid degradation of the entire fusion protein. Peak activity for Cas is sometimes beneficial to reduce off-target effects. Thus, short bursts of high activity are preferred. The present invention is able to provide such peaks. In some senses the system is inducible. In some other senses, the system repressed in the absence of stabilizing ligand and de-repressed in the presence of stabilizing ligand. By means of example, and without limitation, in some embodiments, the DD is ER50. A corresponding stabilizing ligand for this DD is, in some embodiments, 4HT. As such, in some embodiments, one of the at least one DDs is ER50 and a stabilizing ligand therefor is 4HT or CMP8. In some embodiments, the DD is DHFR50. A corresponding stabilizing ligand for this DD is, in some embodiments, TMP. As such, in some embodiments, one of the at least one DDs is DHFR50 and a stabilizing ligand therefor is TMP. In some embodiments, the DD is ER50. A corresponding stabilizing ligand for this DD is, in some embodiments, CMP8. CMP8 may therefore be an alternative stabilizing ligand to 4HT in the ER50 system. While it may be possible that CMP8 and 4HT can/should be used in a competitive matter, some cell types may be more susceptible to one or the other of these two ligands, and from this disclosure and the knowledge in the art the skilled person can use CMP8 and/or 4HT. More than one (the same or different) DD may be present, and may be fused for instance C-terminally, or N-terminally, or even internally at suitable locations. Having two or more DDs which are heterologous may be advantageous as it would provide a greater level of degradation control.

In some embodiments, the nuclease is fused to one or more localization signals, such as nuclear localization sequences (NLSs), such as about or more than about 1, 2, 3, 4, 5, 6, 7, 8, 9, 10, or more NLSs. In some embodiments, the nuclease comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the nuclease comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV; the NLS from nucleoplasmin (e.g. the nucleoplasmin bipartite NLS with the sequence KRPAATKKAGQAKKKK); the c-myc NLS having the amino acid sequence PAAKRVKLD or RQRRNELKRSP; the hRNPA1 M9 NLS having the sequence NQSSNFGPMKGGNFGGRSSGPYGGGGQYFAKPRNQGGY; the sequence RMRIZFKNKGKDTAELRRRRVEVSVELRKAKKDEQILKRRNV of the IBB domain from importin-alpha; the sequences VSRKRPRP and PPKKARED of the myoma T protein; the sequence POPKKKPL of human p53; the sequence SALIKKKKKMAP of mouse c-abl IV; the sequences DRLRR and PKQKKRK of the influenza virus NS1; the sequence RKLKKKIKKL of the Hepatitis virus delta antigen; the sequence REKKKFLKRR of the mouse M×1 protein; the sequence KRKGDEVDGVDEVAKKKSKK of the human poly(ADP-ribose) polymerase; and the sequence RKCLQAGMNLEARKTKK of the steroid hormone receptors (human) glucocorticoid.

In some embodiments, the fusion protein as described herein may comprise a linker between the nuclease and the fusion partner (e.g. functional domain). In some embodiments, the linker is a GlySer linker. Attachment of a functional domain or fusion protein can be via a linker, e.g., a flexible glycine-serine (GlyGlyGlySer) or (GGGS)3 or a rigid alpha-helical linker such as (Ala(GluAlaAlaAlaLys)Ala). Linkers such as (GGGGS)3 are preferably used herein to separate protein or peptide domains. (GGGGS)3 is preferable because it is a relatively long linker (15 amino acids). The glycine residues are the most flexible and the serine residues enhance the chance that the linker is on the outside of the protein. (GGGGS)6 (GGGGS)9 or (GGGGS)12 may preferably be used as alternatives. Other preferred alternatives are (GGGGS)1, (GGGGS)2, (GGGGS)4, (GGGGS)5, (GGGGS)7, (GGGGS)8, (GGGGS)10, or (GGGGS)11. Alternative linkers are available, but highly flexible linkers are thought to work best to allow for maximum opportunity for the 2 parts of the Cas9 to come together and thus reconstitute Cas9 activity. One alternative is that the NLS of nucleoplasmin can be used as a linker. For example, a linker can also be used between the Cas9 and any functional domain. Again, a (GGGGS)3 linker may be used here (or the 6, 9, or 12 repeat versions therefore) or the NLS of nucleoplasmin can be used as a linker between Cas9 and the functional domain.

With particular reference to the CRISPR/Cas system as described herein, besides the Cas protein, in addition or in the alternative, the gRNA and/or tracr (where applicable) and/or tracr mate (or direct repeat) may be modified. Suitable modifications include, without limitation dead guides, escorted guides, protected guides, or guides provided with aptamers, suitable for ligating to, binding or recruiting functional domains (see e.g. also elsewhere herein the reference to synergistic activator mediators (SAM)). Mention is also made of U.S. application 62/091,455, filed, 12-Dec.-14, PROTECTED GUIDE RNAS (PGRNAS); U.S. application 62/096,708, 24-Dec.-14, PROTECTED GUIDE RNAS (PGRNAS); U.S. application 62/091,462, 12-Dec.-14, DEAD GUIDES FOR CRISPR TRANSCRIPTION FACTORS; U.S. application 62/096,324, 23-Dec.-14, DEAD GUIDES FOR CRISPR TRANSCRIPTION FACTORS; U.S. application 62/091,456, 12-Dec.-14, ESCORTED AND FUNCTIONALIZED GUIDES FOR CRISPR-CAS SYSTEMS; all incorporated herein by reference. In certain embodiments, the tracr sequence (where appropriate) and/or tracr mate sequence (direct repeat), may comprise one or more protein-interacting RNA aptamers. The one or more aptamers may be located in the tetraloop and/or stemloop 2 of the tracr sequence. The one or more aptamers may be capapble of binding MS2 bacteriophage coat protein. In certain embodiments, the gRNA (or trace or tracr mate) is modified by truncations, and/or incorporation of one or more mismatches vis-à-vis the intended target sequence or sequence to hybridize with.

By means of further guidance, and without limitation, in certain embodiments, the gRNA is a dead gRNA (dgRNA), which are guide sequences which are modified in a manner which allows for formation of the CRISPR complex and successful binding to the target, while at the same time, not allowing for successful nuclease activity (i.e. without nuclease activity/without indel activity). These dead guides or dead guide sequences can be thought of as catalytically inactive or conformationally inactive with regard to nuclease activity. Several structural parameters allow for a proper framework to arrive at such dead guides. Dead guide sequences are shorter than respective guide sequences which result in active Cas-specific indel formation. Dead guides are 5%, 10%, 20%, 30%, 40%, 50%, shorter than respective guides directed to the same Cas protein leading to active Cas-specific indel formation. Guide RNA comprising a dead guide may be modified to further include elements in a manner which allow for activation or repression of gene activity, in particular protein adaptors (e.g. aptamers) as described herein elsewhere allowing for functional placement of gene effectors (e.g. activators or repressors of gene activity). One example is the incorporation of aptamers, as explained herein and in the state of the art. By engineering the gRNA comprising a dead guide to incorporate protein-interacting aptamers (Konermann et al., “Genome-scale transcription activation by an engineered CRISPR-Cas9 complex,” doi:10.1038/nature14136, incorporated herein by reference), one may assemble a synthetic transcription activation complex consisting of multiple distinct effector domains. Such may be modeled after natural transcription activation processes. For example, an aptamer, which selectively binds an effector (e.g. an activator or repressor; dimerized MS2 bacteriophage coat proteins as fusion proteins with an activator or repressor), or a protein which itself binds an effector (e.g. activator or repressor) may be appended to a dead gRNA tetraloop and/or a stem-loop 2. In the case of MS2, the fusion protein MS2-VP64 binds to the tetraloop and/or stem-loop 2 and in turn mediates transcriptional up-regulation, for example for Neurog2. Other transcriptional activators are, for example, VP64. P65, HSF1, and MyoD 1. By mere example of this concept, replacement of the MS2 stem-loops with PP7-interacting stem-loops may be used to recruit repressive elements.

By means of further guidance, and without limitation, in certain embodiments, the gRNA is an escorted gRNA (egRNA). By “escorted” is meant that the CRISPR-Cas system or complex or guide is delivered to a selected time or place within a cell, so that activity of the CRISPR-Cas system or complex or guide is spatially or temporally controlled. For example, the activity and destination of the CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time. The escorted Cpf1 CRISPR-Cas systems or complexes have a gRNA with a functional structure designed to improve gRNA structure, architecture, stability, genetic expression, or any combination thereof. Such a structure can include an aptamer. Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green flourescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

By means of further guidance, and without limitation, in certain embodiments, the gRNA is a protected guide. Protected guides are designed to enhance the specificity of a Cas protein given individual guide RNAs through thermodynamic tuning of the binding specificity of the guide RNA to target nucleic acid. This is a general approach of introducing mismatches, elongation or truncation of the guide sequence to increase/decrease the number of complimentary bases vs. mismatched bases shared between a target and its potential off-target loci, in order to give thermodynamic advantage to targeted genomic loci over genomic off-targets. In certain embodiments, the guide sequence is modified by secondary structure to increase the specificity of the CRISPR-Cas system and whereby the secondary structure can protect against exonuclease activity and allow for 3′ additions to the guide sequence. In certain embodiments, a “protector RNA” is hybridized to a guide sequence, wherein the “protector RNA” is an RNA strand complementary to the 5′ end of the guide RNA (gRNA), to thereby generate a partially double-stranded gRNA. In an embodiment of the invention, protecting the mismatched bases with a perfectly complementary protector sequence decreases the likelihood of target binding to the mismatched basepairs at the 3′ end. In certain embodiments, additional sequences comprising an extented length may also be present. [0004] Guide RNA (gRNA) extensions matching the genomic target provide gRNA protection and enhance specificity. Extension of the gRNA with matching sequence distal to the end of the spacer seed for individual genomic targets is envisaged to provide enhanced specificity. Matching gRNA extensions that enhance specificity have been observed in cells without truncation. Prediction of gRNA structure accompanying these stable length extensions has shown that stable forms arise from protective states, where the extension forms a closed loop with the gRNA seed due to complimentary sequences in the spacer extension and the spacer seed. These results demonstrate that the protected guide concept also includes sequences matching the genomic target sequence distal of the 20mer spacer-binding region. Thermodynamic prediction can be used to predict completely matching or partially matching guide extensions that result in protected gRNA states. This extends the concept of protected gRNAs to interaction between X and Z, where X will generally be of length 17-20 nt and Z is of length 1-30 nt. Thermodynamic prediction can be used to determine the optimal extension state for Z, potentially introducing small numbers of mismatches in Z to promote the formation of protected conformations between X and Z. Throughout the present application, the terms “X” and seed length (SL) are used interchangeably with the term exposed length (EpL) which denotes the number of nucleotides available for target DNA to bind; the terms “Y” and protector length (PL) are used interchangeably to represent the length of the protector; and the terms “Z”, “E”, “E′” and EL are used interchangeably to correspond to the term extended length (ExL) which represents the number of nucleotides by which the target sequence is extended. An extension sequence which corresponds to the extended length (ExL) may optionally be attached directly to the guide sequence at the 3′ end of the protected guide sequence. The extension sequence may be 2 to 12 nucleotides in length. Preferably ExL may be denoted as 0, 2, 4, 6, 8, 10 or 12 nucleotides in length. In a preferred embodiment the ExL is denoted as 0 or 4 nuleotides in length. In a more preferred embodiment the ExL is 4 nuleotides in length. The extension sequence may or may not be complementary to the target sequence. An extension sequence may further optionally be attached directly to the guide sequence at the 5′ end of the protected guide sequence as well as to the 3′ end of a protecting sequence. As a result, the extension sequence serves as a linking sequence between the protected sequence and the protecting sequence. Without wishing to be bound by theory, such a link may position the protecting sequence near the protected sequence for improved binding of the protecting sequence to the protected sequence. Addition of gRNA mismatches to the distal end of the gRNA can demonstrate enhanced specificity. The introduction of unprotected distal mismatches in Y or extension of the gRNA with distal mismatches (Z) can demonstrate enhanced specificity. This concept as mentioned is tied to X, Y, and Z components used in protected gRNAs. The unprotected mismatch concept may be further generalized to the concepts of X, Y, and Z described for protected guide RNAs.

In certain embodiments, any of the nucleases, including the modified nucleases as described herein, may be used in the methods, compositions, and kits according to the invention. In particular embodiments, nuclease activity of an unmodified nuclease may be compared with nuclease activity of any of the modified nucleases as described herein, e.g. to compare for instance off-target or on-target effects. Alternatively, nuclease activity (or a modified activity as described herein) of different modified nucleases may be compared, e.g. to compare for instance off-target or on-target effects.

In on embodiment, the present invention provides kits of parts for the practice of the methods according to the invention. The kits of the invention preferably include one or more containers each containing a different component of the kit, such as a container comprising a solid support comprising one or more nucleic acid molecules immobilized thereon, a container comprising an agent capable of inducing a nucleic acid modification, preferably comprising a targeted nuclease, a solid support comprising a plurality of first and second oligonucleotides immobilized thereto, a container comprising a first adapter comprising a sequence that is able to hybridize to said first immobilized oligonucleotides and a container comprising a second adapter comprising a sequence that is complementary to a sequence that is able to hybridize to said immobilized second oligonucleotides, and/or a container comprising one or more nucleic acid molecules, a DNA or RNA polymerase, a restriction enzyme, a ligase, an exonuclease, a mixture of nucleotides or labelled nucleotides.

Associated with such kit of parts or such container(s) can be various written materials such as instructions for use, or a notice in the form prescribed by a governmental agency regulating the manufacture, use or sale of the kits. Preferably, the kit of parts comprises instructions for use.

The present invention will be further illustrated in the following Examples which are given for illustration purposes only and are not intended to limit the invention in any way.

### EXAMPLES

### Example 1. Validation of an Assay for Detection of Cut DNA Immobilized on a Flow Cell

Materials:
Cas9 protein (from NEB)
In vitro transcribed and purified sgRNA (T7 HiScribe from NEB, then Megaclear)
IVT kit (e.g. NEB T7 Hiscribe)
Purification kit (e.g. Megaclear)
VEGFA3 sgRNA IVT primer
EMX1.3 sgRNA IVT primer
Illumina Miseq v2 50 cycle kit

Substrate for Cutting:

For validation of reaction chemistry, amplicons containing known Cas9 targets and off-targets, and restriction endonuclease targets (blunt or sticky end) for positive controls are mixed with negative control amplicons containing no target site or PAM mutations. It is expected that on and off-target cutting from these amplicon pools will generalize to whole genome applications with minimal modification.

Amplicon composition:
P5 and P7 5′ and 3′ termination sequences (contain sequences for: flow cell binding, bridge amplification, paired end sequencing, and index for sample discrimination)
70 bp of intervening sequence between P5 and P7 comprising either:
1. 20 nt Cas9 on/off target sites, PAM, and flanking sequence from human genome
2. sequences from (1) with mutated PAMs to eliminate Cas9 targeting
3. EcoRV and EcoRI target sites with synthetic flanking sequence

Target DNA sequences:
EMX1.3 T7 sgRNA template:
gaaatTAATACGACTCACTATAgGAGTCCGAGCAGAAGAAGAA
gttttagagctaGAAAtagcaagttaaaataaggctagtccgt
tatcaacttgaaaaagtggcaccgagtcggtgcttt
VEGFA3 T7 sgRNA template:
gaaatTAATACGACTCACTATAgGGTGAGTGAGTGTGTGCGTG
gttttagagctaGAAAtagcaagttaaaataaggctagtccgt
tatcaacttgaaaaagtggcaccgagtcggtgcttt.
T7 (sense direction):
gaaatTAATACGACTCACTATA.

Adapters for DSB Labeling:

Pilot chemistry: For initial testing of our reaction chemistry, fluorescently tagged adapters are used ligated specifically to DSBs generated from targeted DNA manipulation on the flowcell. The chemistry of the fluorescent labeling strategy differs slightly from the chemistry used for sequencing-based readout of DNA manipulation activity. For fluorescent readout of DSBs introduced by targeted DNA manipulation, no phosphate will be included on the adapter, resulting in ligation of the adapter only to 5′ phosphate terminated DSBs (uncut clusters will not contain 5′ phosphates). The same chemistry will not be used in sequencing applications, since this chemistry will leave a nick between the ligated adapter and the 3′ end of the flow-cell immobilized strand on the cut cluster resulting in loss of the adapter upon strand denaturation prior to sequencing readout of the DSB.

Production chemistry: Sequence-based readout adapters are generated containing a single 5′ phosphorylated terminus on the strand complementary to the primer for DSB sequencing readout. This will result in ligation of adapter to all cut and uncut clusters and sequencing of all clusters. This will take no additional time compared to sequencing of only uncut clusters, and cut/uncut clusters will be easily distinguished based on the presence or absence of terminating adapters present on uncut products. The sequencing of all clusters may also be useful for quality control.

Alternatively, uncut clusters can be treated prior to cutting for the addition of a single 3′ phosphorothioate nucleotide overhang at the DNA terminus. This prevents blunting of uncut products following manipulation of DNA clusters. Hence, clusters containing a 3′ overhang would not be capable of ligating DSB labeling adapters, resulting in specific labeling of DSBs enabling serial fluorescent readout of fluorophore tagged adapter ligation events and terminal sequencing of manipulated nucleotide clusters. Such applications enable the serialization of cutting and ligation allowing simultaneous investigation of the efficiency and specificity of nucleotide manipulation.

Adapter composition for fluorescent tagging pilot assay:
Top strand T7 promoter sequence with a fluorescent molecule at the 5′ terminus and optionally) a sample barcode and/or UMI at the 3′ terminus.
T7 as the root sequence of the adapter facilitates generation of RNA for additional sequencing of labeled ends for validation/confirmation.
T7 sequence could be replaced by alternative nucleotide sequence.
Fluorescent termini will be labeled with either Fluorescein (green) and Cy3 (red)
   Enables serial DNA manipulation and tagging of multiple independent targets.
No 5′ phosphate will be included on the T7 top strand-complementary oligo to facilitate for specific fluorescent labeling of manipulated nucleotide clusters containing exposed 5′ phosphates
5′ phosphates could be included for tagging of all DNA clusters on the flow cell.
See table 1 for specific adapter sequences:

TABLE 1

| | | | bar- | total |
|---|---|---|---|---|
| Fwd | 5′ mod | T7 promoter | code | sequence |
| sw_fwd_1_ | /5Alex | TAATACGACT | GTCG | /5Alex488N/ |
| green_T7 | 488N/ | CACTATAGGG | TCGC | TAATACGACTC |
| | | | | ACTATAGGGGT |
| | | | | CGTCGC |
| sw_fwd_2_ | /5Cy3/ | TAATACGACT | ACGA | /5Cy3/TAATA |
| red_T7 | | CACTATAGGG | CCGC | CGACTCACTAT |
| | | | | AGGGACGACCG |
| | | | | C |
| | | TAATACGACT | TGAT | TAATACGACTC |
| | | CACTATAGGG | GCGC | ACTATAGGGTG |
| | | | | ATGCGC |
| | | TAATACGACT | CATC | TAATACGACTC |
| | | CACTATAGGG | AATC | ACTATAGGGCA |
| | | | | TCAATC |
| | | revcomp | | |
| | | (barcode + | | total |
| Rev | 5′ mod | T7 promoter) | | sequence |
| sw_rev_1_ | | GCGACGACCC | | GCGACGACCCC |
| green_T7 | | CTATAGTGAG | | TATAGTGAGTC |
| | | TCGTATTA | | GTATTA |
| sw_rev_2_ | | GCGGTCGTCC | | GCGGTCGTCCC |
| red_T7 | | CTATAGTGAG | | TATAGTGAGTC |
| | | TCGTATTA | | GTATTA |
| sw_rev_1_ | /5phos/ | GCGACGACCC | | /5phos/GCGA |
| green_T7_ | | CTATAGTGAG | | CGACCCCTATA |
| phos | | TCGTATTA | | GTGAGTCGTAT |
| | | | | TA |
| sw_rev_2_ | /5phos/ | GCGGTCGTCC | | /5phos/GCGG |
| red_T7_ | | CTATAGTGAG | | TCGTCCCTATA |
| phos | | TCGTATTA | | GTGAGTCGTAT |
| | | | | TA |

Adapter Composition for Sequencing-Based Production Assay (optionally Including Fluorescence):
Top strand primer sequence (optionally including: fluorescent molecule at the 5′ terminus and a sample barcode and/or UMI at the 3′ terminus as specified above).
5′ phosphates could be included for tagging of all DNA clusters on the flow cell.

Pilot Assay Specifications:

### Flow Cell Loading+Miseq Run

1. Load Miseq with about 2 pM of library (compared to 12-14 pM @ normal loading density)
   Library will actually contain a mix of pilot assay substrates specified above
2. Generate clusters
3. Sequence clusters using with 25 bp paired end reads and 8 bp index (50 cycle kit should be able to accommodate)
4. After sequencing complete, remove chip from Miseq (be sure to replace with another one to allow washing) Completing second strand synthesis on flow cell
5. If sequencing read covers complete length of amplicon, complete dsDNA will be present on the flow cell after sequencing (skip step 6-8).
   If second strand is not completed, second strand synthesis will be required for dsDNA manipulation assays (steps 6-8).
6. Wash flow cell 2-5× with second strand synthesis buffer solution using input and output gaskets on flow cell for injection of solution and collection of waste.
7. Use High-fidelity polymerase, Klenow, or DNA polymerase I (with dNTPs) to complete second strand synthesis.
8. Wash 3× with 1× Cutsmart, or equivalent DNA manipulation buffer

### Cas9 Treatment

9. Incubate 5 min with Cas9 buffer to equilibrate buffer
10. Mix together Cas9 protein and sgRNA in Cas9 buffer; put onto flow cell
11. Incubate 37C for 20 min-3 hours (optimize for saturated on/off-target manipulation)
12. Wash 3× with Cutsmart, or equivalent buffer at RT

### Getting Rid of Cas9

13. Make Proteinase K solution with Proteinase K, Triton, and cutsmart or equivalent buffer
14. Add solution to flowcell and incubate at 25-37C for 30 min
15. Wash flowcell thoroughly or add Proteinase K inhibitor to inactivate and remove

### Blunting Cas9 Induced DSBs

16. Wash 3× with Cutsmart at RT
17. Wash 1× with 1× Quick blunting buffer at RT
18. Anneal DNA by moving back to RT slowly
19. Make Quick Blunting enzyme solution and put into flow cell
20. Incubate at RT for 30 min

### Prepare Pilot Assay Adapters

21. Anneal pilot adapters (as specified above)
   example conditions: incubate top strand and bottom strand adapter oligos at 95C to 4C at 2% ramp rate on thermocyclers (˜0.1C/sec) in 1× T4 ligase buffer

### Ligation of Pilot Assay Adapters to Manipulated DNA Ends

22. Wash 3× with Cutsmart at RT
23. Wash 1× with T4 DNA Ligase/Quick ligase buffer
24. Add T4 Ligase/Quick Ligase enzyme mixture containing annealed pilot assay adapters
25. Ligate (overnight at 16C, or 10 min-2 h at 25C)

### Final Wash

26. Wash flow cell thoroughly to remove excess adapter
   example: Hi salt wash to bind and remove of excess DNA
27. Choose final buffer for imaging that is not autofluorescent

### Imaging

28. Clusters should be about 1 um in diameter; these should be visible on a fluorescent microscope using a 20×-100× objective
   T7 Transcription & RNA Collection (optional for Additional Sequencing Validation)
   Inclusion of T7 promoter on fluorescent adapter facilitates the generation of RNA transcripts from ligation events at and preparation of a sequencing library that contains the sequences of the breaks
29. Flush flow cell with 1× in vitro transcription buffer
30. Flow in 1× NEB Hiscribe T7 enzyme mix
31. Incubate 4 hours at 37C
32. Collect solution

### DNase Digest

33. Incubate collected solution with DNase (preferred: Turbo DNase)
34. Purify after DNase treatment using column (either Qiagen RNeasy minelute or Megaclear depending on yield)
35. Run out purified RNA on gel
36. Prepare sequencing library from purified RNA using NEBNext small RNA library prep.
   [Can repeat the Cas9 cutting treatment and use a different fluorescent oligo for multiplexing of additional targets in a single assay].

Production Assay Specifications:

### Flow Cell Loading+Nextseq Run

1. Load Nextseq with about 2-3 pM of whole genome library of interest.
2. Generate clusters.
3. optional: Sequence clusters using with 35-150 bp paired end reads and 8 bp index (75-300 nt Nextseq high output kit).
4. After sequencing complete, perform additional reactions on Nextseq platform
   optionally, reactions could be performed with the chip removed from the Nextseq.
      Proceed with steps 5-26 as specified above substituting the production assay adapter for pilot assay adapter.
27. Sequence clusters using primer included on production assay adapter.

### Example 2—Enrichment Protocol (Linear)

### Materials:

Qiagen Blood and tissue DNA Midi
Hiscribe T7 Quick High Yield RNA Synthesis Kit (NEB, E2050S)
NEBNext Ultra II End Repair/dA-Tailing Module
NEB quick ligation kit
NEB Next high fidelity master mix

### Sample Harvest:

1. Harvest 2×T225 flasks (˜100M cells) with trypsin.
2. Extract gDNA using Qiagen Blood and tissue DNA Midi
   Adapter and sgRNA T7 Annealing:
1. Mix Adapter TS and BS oligos
   1 ul TS adapter oligo (100 uM)
   1 ul BS adapter oligo (100 uM)
   1 ul TaqB or equivalent PCR buffer
   7 ul H2O
Total: 10 ul
Pairs to be Annealed:
   sgRNA T7 Annealing:
T7 TS GGG primer oligo (Vd_0374)
T7-GGG-sgRNA BS oligo

### RA3 Adapter:

ISceI-RA3_fwd_phos_PS
ISceI-RA3_rev_PS

### RA5 Adapter:

RA5_fwd_phos_PS
RA5_rev_PS_PS_T

2. Annealing conditions
5 min @ 95C
cool to 4C, ramp 5C/min
   T7 Transcription (sgRNA Annealed Oligos): Transcription
1. T7 transcription reaction
   Small RNA synthesis protocol
      16 ul Nuclease free H2O
      10 ul NTP Buffer Mix
      2 ul template annealed product (˜1 ug)
      2 ul T7 RNA Pol Mix
   Total: 30 ul
2. Reaction conditions
   37C, 3 h
   4C

3. Store at −80

### Cleanup

1. Add 100 ul RNA XP spri beads to each 30 ul RNA sample.
2. Add 90 ul isopropanol, mix well.
3. Incubate 5 min at RT.
4. Place on magnet for ˜1 min until solution clears.
5. Aspirate supernatant
6. Rinse 2× with 200 ul 85% EtOH
7. Dry ˜5 min (until beads no longer look shiny)
8. Elute in 50 ul H2O
   gDNA Shearing and End Prep:

### Shearing:

1. Prepare for shearing (8 rxn per tube)
   500 ng gDNA in H2O (500 ng=˜75 k genomic coverage)
   7 ul NEBNext Ultra II End Prep Reaction Buffer
   (Check and make sure that buffer does not contain PEG)
   Total: 67 ul
2. Sonicate sample as follows:
   1. Sonicate on high power 30 s on/off 15 min (Bioruptor)
   2. Move directly to blunting and A-tailing

### Blunting and A-Tailing:

1. To the sheared gDNA mixture add:
   3 ul NEBNext Ultra II End Prep Enzyme Mix
   Total: 70 ul
3. Reaction conditions
   30 min @ 20C
   30 min @ 65C
   Hold at 4C
4. Spri reaction contents (2.0×) or column purify
   Elute in 30 ul H2O

### Ligation:

Try: quick vs. blunt-TA and adapter comparison Expt: 160703 shows that quick and blunt-TA perform roughly equivalently for this reaction
Due to higher conc., quick may outperform blunt-TA at increased molar amounts of sample
   1. Quick ligation reaction
   26 ul Eluted A-tailed product
   30 ul Quick ligase buffer
   2 ul Quick ligase
   2 ul (2.5 uM) Annealed adapter
      RA3_fwd_phos_PS
      RA3_rev_5′InvddT_PS_PS_T
   Total: 60 ul
      2. Reaction conditions
   25C, 30 min
   4C
      3. Spri reaction contents (2.0×) or column purify
   NOTE: calculate based on PEG already in ligase buffer
   Elute in 25 ul H2O

### Exonuclease Digestion:

### 1× Exonuclease I Reaction Buffer:

67 mM Glycine-KOH

6.7 mM MgCl2

10 mM β-ME

pH 9.5 @ 25C

### Exonuclease I Digestion:

(3′->5′ digestion of ssDNA)
1. Reaction
   23 ul Ligation product
   5 ul 10× Exonuclease I buffer
   1 ul Exonuclease I
   1 ul Lambda Exonuclease
Total: 50 ul (20 ul H2O)
2. Reaction conditions
   37C, 1 h
   80C, 15 min
   4C
3. Spri 2.0× to Keep all Products
   Elute in 25 ul H2O
      PCR Enrichment (May be optional with Increased Input gDNA):
      Expt 160703 shows that use of short primer: ISceI-PP_universal_PCR_fwd results in byproduct formation

### Use Primer:

ISceI-RA3_rev_PS
G*A*G*T*T*TAGGGATAACAGGGTA
ATCCTTGGCACCCGAGAATTCCA*T

NOTE: for single primer PCR, need primer of
>=Tm of handle on either side of amplicon
High Tm of handles may result in strong cis hairpin formation
Formation of these structures would be accelerated for shorter amplicons
Observe biasing up of library size consistent with above
Shorter handles=hairpin breathing @ T anneal->primer annealing
1. NEB Next reaction
   23 ul Exonuclease digested product
   25 ul NEB Next HF MM
   2 ul (10 uM) ISceI-RA3_rev_PS
Total: 50 ul
2. Reaction conditions
   30 s @ 98C
   loop 15×
      10 s @ 98C
      30 s @ 65C
      2 min @ 72C
   5 min @ 72C
   4C forever
3. column purify
   Elute in 25 ul H2O
      Column purify here to remove all preceding reagents Ensure that no residual exo activity from exo digest
or polymerase carries into manipulation step

### Questions of Under Extended Products:

Are under extended products contributing to bkg noise
Should exo be performed after PCR with no heat inactivation
Then column purify

### Questions of Mechanical Disrupt Between Enrich and Post-Cut Ligation:

Does column purification introduce mechanical DSBs
Is the mechanical disruption of column greater than spri

### Targeted DNA Manipulation:

### Cas9 Digestion:

(5′->3′ digestion of dsDNA)
1. Reaction (Consider buffer optimization)
   5 ug Enrichment product
   1-3 ug sgRNA/crRNA
   5 ul 10× NEB buffer 3
   0.225 ul 1M DTT
   100-300 ng enzyme
   Total: 50 ul
2. Reaction conditions
   37C, 8 h
   4C
3. Spri reaction contents (2.0×) or column purify
   Elute in 40 ul H2O
      Try: Spri Vs. minElute
+RNaseA 10 min->+pK 10 min->minElute
no RNaseA, no Pk->spri
Test necessity of removing Cas9 bound at cut ends for successful DSB ligation

### Post Cas9 End Prep:

### Blunting and A-Tailing:

1. Prepare following reaction:
   35 ul cut gDNA
   7 ul NEBNext Ultra II End Prep Buffer
   3 ul NEBNext Ultra II End Prep Enzyme Mix
   Total: 70 ul (25 ul H2O)
2. Reaction conditions
   30 min @ 20C
   30 min @ 65C
   Hold at 4C
3. Spri reaction contents (2.0×) or column purify
   Elute in 25 ul H₂O
      Test: Efficiency of Pre-Ligation Vs. Post-Ligation I-SceI

### Ligation:

1. Quick ligation reaction
   23 ul Eluted A-tailed product
   30 ul Quick ligase buffer
   5 ul Quick ligase
   2 ul (2.5 uM) Annealed adapter
   RA5_fwd_phos_PS
   RA5_rev_PS_PS_T
Total: 60 ul
2. Reaction conditions
   25C, 30 min
   4C
3. Spri reaction contents (1.0×)×2 to Remove Adapter
   Elute in 25 ul H₂O

### I-SceI Digest:

1. I-SceI reaction
   23 ul library product
   5 ul 10× buffer tango
   3 ul I-SceI
Total: 50 ul
2. Reaction conditions
   37C, 1 h
   4C
3. Spri reaction contents (1.0×) to Remove Adapter
   Elute in 25 ul H2O

### T7 Transcription (T7-RA5 Ligated Prodcts):

1. T7 transcription reaction

### Small RNA Synthesis Protocol

18 ul ligation product
10 ul NTP Buffer Mix
2 ul T7 RNA Pol Mix
Total: 30 ul
2. Reaction conditions
   37C, 2-8 h
   4C
3. Proceed Directly to DNaseI Reaction
   30 ul T7 reaction
   68 ul H2O
   2 ul DNase I
   Total: 100 ul
4. Incubate 37C, 15 Min
5. Add 0.75 ul 0.5M EDTA (Final: 5 mM)
6. Heat Inactivate 75C, 10 Min
7. 2.0× Spri reaction contents (RNA Clean Spri)
   Elute in 25 ul H₂O

### Reverse Transcription (T7 Products):

1. Superscript III Annealing Reaction
   Xul up to 5 ug T7 product
   1 ul (2 uM) RTP (increase to 10 uM based on illumin smRNA)
   1 ul annealing buffer
   Total: 8 ul
2. Annealing conditions
   65C, 5 min
   immediately to 4C, >=1 min
3. Superscript III rT Reaction
   Add to annealing product:
      10 ul First-strand reaction mix
      2 ul Superscript III RNaseOUT mix
   Total: 20 ul
4. Annealing conditions
   50C, 50 min
   85C, 5 min
   4C
5. (optional) add RNaseH step
   RNA should be destroyed during PCR, probably do not need
6. Spri reaction contents (1.0×)
   Elute in 25 ul H2O

### PCR Enrichment:

4. NEB Next Reaction
   21 ul Ligation product
   25 ul NEB Next HF MM
   2 ul (10 uM) RA3 P7 extension primer (RP1)
   2 ul (10 uM) RA5 P5 extension primer (RPI1)

### Total: 50 ul

5. Reaction conditions
   30 s @ 98C
   loop 12×
   10 s @ 98C
   30 s @ 65C
   30 s @ 72C
   5 min @ 72C
   4C forever
6. Spri reaction contents (2.0×) or column purify
   Elute in 40 ul H2O

### Example 3—Enrichment Protocols (Circular)

### Materials:

Qiagen Blood and tissue DNA Midi
Hiscribe T7 Quick High Yield RNA Synthesis Kit (NEB, E2050S)
NEBNext Ultra II End Repair/dA-Tailing Module
NEB quick ligation kit
NEB Next high fidelity master mix

### Sample Harvest:

1. Harvest 2×T225 flasks (˜100M cells) with trypsin.
2. Extract gDNA using Qiagen Blood and tissue DNA Midi
   Adapter and sgRNA T7 Annealing:
1. Mix Adapter TS and BS Oligos
   1 ul TS adapter oligo (100 uM)
   1 ul BS adapter oligo (100 uM)
   1 ul TaqB or equivalent PCR buffer
   7 ul H2O
   Total: 10 ul

Pairs to be Annealed:

sgRNA T7 Annealing:
T7 TS GGG primer oligo (Vd_0374)
T7-GGG-sgRNA BS oligo

RA3 Adapter:
ISceI-RA3_fwd_phos_PS
ISceI-RA3_rev_PS

RA5 Adapter:
RA5_fwd_phos_PS
RA5_rev_PS_PS_T
2. Annealing conditions
   5 min @ 95C
   cool to 4C, ramp 5C/min
      T7 Transcription (sgRNA Annealed Oligos):

Transcription
1. T7 transcription reaction
   Small RNA synthesis protocol
   1. 16 ul Nuclease free H2O
   2. 10 ul NTP Buffer Mix
   3. 2 ul template annealed product (˜1 ug)
   4. 2 ul T7 RNA Pol Mix
      Total: 30 ul
2. Reaction conditions
   37C, 3 h
   4C
3. Store at −80

### Cleanup

1. Add 100 ul RNA XP spri beads to each 30 ul RNA sample.

2. Add 90 ul isopropanol, mix well.

3. Incubate 5 min at RT.

4. Place on magnet for ˜1 min until solution clears.

5. Aspirate supernatant

6. Rinse 2× with 200 ul 85% EtOH

7. Dry ˜5 min (until beads no longer look shiny)

8. Elute in 50 ul H2O

gDNA Shearing and End Prep:

Shearing:
1. Prepare for shearing (8 Rxn Per Tube)
   5 ug gDNA in H2O (500 ng=˜75 k genomic coverage)
   7 ul NEBNext Ultra II End Prep Reaction Buffer
   (Check and make sure that buffer does not contain PEG)
   Total: 60 ul
2. Sonicate sample as follows:
   1. Sonicate on high power 30 s on/off 15 min (Bioruptor)
   2. Move directly to blunting and A-tailing

### Blunting:

1. To the sheared gDNA mixture add:
   7 ul 1 mM dNTP
   3 ul NEBNext Ultra II End Prep Enzyme Mix
   Total: 70 ul
2. Reaction conditions
   60 min @ 25C
   Hold at 4C
3. Spri reaction contents (2.0×) or column purify
   Elute in 30 ul H2O

### Ligation:

Try: quick vs. blunt-TA and adapter comparison
Expt: 160703 shows that quick and blunt-TA perform roughly equivalently for this reaction
Due to higher conc., quick may outperform blunt-TA at increased molar amounts of sample
1. Quick ligation reaction
   26 ul Eluted A-tailed product
   30 ul Quick ligase buffer
   2 ul Quick ligase
   Total: 60 ul
2. Reaction conditions
   25C, 30 min
   4C
3. Spri reaction contents (2.0×) or column purify
   NOTE: calculate based on PEG already in ligase buffer
   Elute in 25 ul H2O

### Exonuclease Digestion:

1× Exonuclease I Reaction Buffer:
67 mM Glycine-KOH
6.7 mM MgCl2
10 mM β-ME
pH 9.5 @ 25C

Exonuclease I Digestion:

(3′->5′ digestion of ssDNA)
1. Reaction
   23 ul Ligation product
   5 ul 10× Exonuclease I buffer
   1 ul Exonuclease I
   1 ul Lambda Exonuclease
   Total: 50 ul (20 ul H2O)
2. Reaction conditions
   37C, 1 h
   4C
3. Spri 2.0× to Keep all Products
   Elute in 25 ul H2O
      Column purify here to remove all preceding reagents
Ensure that no residual exo activity from exo digest or polymerase carries into manipulation step
   Questions of under extended products:
   Are under extended products contributing to bkg noise
   Should exo be performed after PCR with no heat inactivation
   Then column purify
      Questions of mechanical disrupt between enrich and post-cut ligation:
   Does column purification introduce mechanical DSBs
   Is the mechanical disruption of column greater than spri

### Targeted DNA Manipulation:

Cas9 Digestion:

(5′->3′ digestion of dsDNA)
1. Reaction (Consider Buffer Optimization)
   5 ug Enrichment product
   1-3 ug sgRNA/crRNA
   5 ul 10× NEB buffer 3
   0.225 ul 1M DTT
   100-300 ng enzyme
   Total: 50 ul
2. Reaction conditions
   37C, 8 h
   4C
3. Spri reaction contents (2.0×) or column purify
   Elute in 40 ul H2O
Try: spri vs. minElute
   +RNaseA 10 min->+pK 10 min->minElute
   no RNaseA, no Pk->spri
Test necessity of removing Cas9 bound at cut ends for successful DSB ligation

### Post Cas9 End Prep:

Blunting and A-tailing:
1. Prepare following reaction:
   35 ul cut gDNA
   7 ul NEBNext Ultra II End Prep Buffer
   3 ul NEBNext Ultra II End Prep Enzyme Mix
   Total: 70 ul (25 ul H2O)
2. Reaction conditions
   30 min @ 20C
   30 min @ 65C
   Hold at 4C
3. Spri reaction contents (2.0×) or column purify
   Elute in 25 ul H2O

Test: efficiency of pre-ligation vs. post-ligation I-SceI

### Ligation:

1. Quick ligation reaction
   i. 23 ul Eluted A-tailed product
   ii. 30 ul Quick ligase buffer
   iii. 5 ul Quick ligase
   iv. 2 ul (2.5 uM) Annealed adapter
      RA5_fwd_phos_PS
      RA5_rev_PS_PS_T
   Total: 60 ul
2. Reaction conditions
   25C, 30 min
   4C
3. Spri reaction contents (1.0×)×2 to Remove Adapter
   Elute in 25 ul H2O
      I-SceI digest:
1 I-SceI Reaction
   23 ul library product
   5 ul 10× buffer tango
   3 ul 1-SceI
   Total: 50 ul
2. Reaction conditions
   37C, 1 h
   4C
3. Spri reaction contents (1.0×) to Remove Adapter
   Elute in 25 ul H2O

### T7 Transcription (T7-RA5 Ligated Products):

1. T7 transcription reaction
   Small RNA synthesis protocol
      18 ul ligation product
      10 ul NTP Buffer Mix
      2 ul T7 RNA Pol Mix
      Total: 30 ul
2. Reaction conditions
   37C, 2-8 h
   4C
3. Proceed Directly to DNaseI Reaction
   30 ul T7 reaction
   68 ul H2O
   2 ul DNase I
   Total: 100 ul
4. Incubate 37C, 15 Min
5. Add 0.75 ul 0.5M EDTA (Final: 5 mM)
6. Heat Inactivate 75C, 10 Min
7. 2.0× Spri reaction contents (RNA Clean Spri)
   Elute in 25 ul H2O

### Reverse Transcription (T7 Products):

1. Superscript III Annealing Reaction
   Xul up to 5 ug T7 product
   1 ul (2 uM) RTP (increase to 10 uM based on illumin smRNA)
   1 ul annealing buffer
Total: 8 ul
2. Annealing conditions
   65C, 5 min
   immediately to 4C, >=1 min
3. Superscript III rT Reaction
   Add to annealing product:
      10 ul First-strand reaction mix
      2 ul Superscript III RNaseOUT mix
      Total: 20 ul
4. Annealing conditions
   50C, 50 min
   85C, 5 min
   4C
5. (optional) add RNaseH step
   RNA should be destroyed during PCR, probably do not need
6. Spri reaction contents (1.0×)
   Elute in 25 ul H2O

### PCR Enrichment:

1. NEB Next Reaction
   21 ul Ligation product
   25 ul NEB Next HF MM
   2 ul (10 uM) RA3 P7 extension primer (RP1)
   2 ul (10 uM) RA5 P5 extension primer (RPI1)
   Total: 50 ul
2. Reaction conditions
   a. 30 s @ 98C
   b. loop 12×
      10 s @ 98C
      30 s @ 65C
      30 s @ 72C
   c. 5 min @ 72C
   d. 4C forever
3. Spri reaction contents (2.0×) or column purify
   Elute in 40 ul H2O

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method for detecting a nucleic acid modification, the method comprising:
i. contacting one or more nucleic acid molecules immobilized on a solid support (immobilized nucleic acid molecules) with an agent capable of inducing a nucleic acid modification; and
ii. sequencing at least part of said one or more immobilized nucleic acid molecules using a primer specifically binding to a primer binding site, said part comprising said nucleic acid modification,
wherein said method comprises attaching an adapter comprising said primer binding site to said one or more immobilized nucleic acid molecules following said contacting step and prior to sequencing in step ii, or
wherein said one or more immobilized nucleic acid molecules that are contacted with said agent comprise an adapter comprising said primer binding site.

2. The method according to claim 1 wherein said nucleic acid is RNA or DNA.

3. The method according to claim 1 or 2 wherein said nucleic acid is single stranded or double stranded.

4. The method according to any one of claims 1-3 wherein said one or more nucleic acid molecules comprise genomic DNA (gDNA).

5. The method according to any one of claims 1-4 wherein said one or more nucleic acid molecules comprise gDNA fragments.

6. The method according to claim 4 or 5 wherein said gDNA is obtained from a patient in need of genome editing.

7. The method according to any one of claims 1-6 wherein said nucleic acid modification is selected from the group consisting of methylation, a mutation, a deletion, an insertion, a replacement, a ligation, a digestion, a strand break and a recombination.

8. The method according to claim 7 wherein said strand break is a nick, a single strand break (SSB) or a double strand break (DSB).

9. The method according to claim 8 wherein said nucleic acid is double stranded, said nucleic acid modification is a nick and said method further comprises contacting said one or more immobilized nucleic acid molecules with a S1 nuclease subsequent to said contacting with an agent capable of inducing a nick.

10. The method according to any one of claims 1-9 wherein said agent comprises a chemical agent or an enzyme.

11. The method according to any one of claims 1-10 wherein said agent is selected from the group consisting of a (viral) integrase, a recombinases, a transposase, an argonaute, a cytidine deaminase, a retron and a group II intron.

12. The method according to claim 10 wherein said enzyme comprises a nuclease.

13. The method according to any one of claims 1-10 and 12 wherein said agent comprises a targeted nuclease complex.

14. The method according to claim 13 wherein said targeted nuclease complex comprises a ZFN, TALEN or CRISPR-Cas.

15. The method according to any one of claims 12-14 wherein said nuclease is selected from the group consisting of Cas9, Cpf1, C2c1, C2c2, C2c3, a group 29 nuclease, a group 30 nuclease and derivatives thereof.

16. The method according to any one of claims 1-15 wherein the method further comprises amplification of said one or more immobilized nucleic acid molecules prior to said contacting with an agent capable of inducing a nucleic acid modification.

17. The method according to any one claims 1-16 wherein the method further comprises sequencing at least part of said one or more immobilized nucleic acid molecules prior to said contacting with an agent capable of inducing a nucleic acid modification.

18. The method according to claim 17 wherein the method further comprises comparing the sequences obtained prior to and subsequent to said contacting with an agent capable of inducing a nucleic acid modification.

19. The method according to any one of claims 13-18 wherein said one or more immobilized nucleic acid molecules are incubated with a plurality of targeted nuclease complexes.

20. The method according to any one of claims 1-19 wherein the nucleic acid molecules are attached to said solid support via a chemical or protein linker.

21. The method according to claim any one of claims 1-20, wherein said solid support comprises a plurality of chemical or protein moieties and the method comprises, prior to said contacting step i, allowing one or more nucleic acid molecules flanked by a first and a second adapter, wherein at least one of said adapters comprises a chemical or biological moiety capable of binding to said chemical or biological moieties of said solid support, to bind to said solid support.

22. The method according to any one of claims 1-21 wherein said method comprises prior to said contacting step i:
amplification of one or more nucleic acid molecules flanked by a first adapter comprising a first primer binding site and a second adapter comprising a second primer binding site in a droplet using primers specifically binding to said primer binding sites, wherein at least one of said primers comprises a chemical or biological moiety capable of binding to a solid support; and
allowing said amplified nucleic acid molecules to bind to said solid support.

23. The method according to claim 22, wherein said amplification comprises emulsion amplification.

24. The method according to any one of claims 1-19 wherein the method comprises allowing one or more nucleic acid molecules flanked by a first and a second adapter to hybridize to one of a plurality of first or second oligonucleotides that are immobilized on a solid support, whereby said first adapter comprises a sequence that is able to hybridize to said first immobilized oligonucleotides and said second adapter comprises a sequence that is complementary to a sequence that is able to hybridize to said immobilized second oligonucleotides.

25. The method according to any one of claims 1-24 wherein the method further comprises amplifying said one or more immobilized nucleic acid molecules, thereby producing a plurality of immobilized nucleic acid molecules.

26. The method according claim 25 wherein said amplifying comprises bridge amplification.

27. The method according to claim 26 wherein the method comprises:
a) allowing one or more nucleic acid molecules flanked by said first and second adapter to hybridize to one of said plurality of first or second immobilized oligonucleotides, whereby said first adapter comprises a sequence that is able to hybridize to said first immobilized oligonucleotides and said second adapter comprises a sequence that is complementary to a sequence that is able to hybridize to said immobilized second oligonucleotides;
and the bridge amplification comprises:
b) extending said first oligonucleotide with a polymerase whereby said one or more single stranded nucleic acid molecules flanked by a first and a second adapter are used as a template;
c) removing said one or more single stranded nucleic acid molecules flanked by a first and a second adapter used as a template resulting in one or more single stranded immobilized nucleic acid molecules;
d) hybridizing said one or more single stranded immobilized nucleic acid molecules to one of said plurality of immobilized second oligonucleotides;
e) extending said second oligonucleotide with a polymerase resulting in one or more double stranded immobilized nucleic acid molecules;
f) denaturing said one or more double stranded immobilized nucleic acid molecules to produce a plurality of immobilized single stranded nucleic acid molecules; and
g) repeating steps d-f at least once.

28. The method according to any one of claims 1-27 wherein said modification comprises a break and wherein said method comprises attaching an adapter comprising said primer binding site to said one or more immobilized nucleic acid molecules following step and prior to sequencing in step iii.

29. The method according to any one of claims 1-28 wherein said one or more immobilized nucleic acid molecules are unphosphorylated.

30. The method according to any one of claims 1-29 wherein said one or more immobilized nucleic acid molecules are treated with phosphatase prior to said contacting with an agent capable of inducing a nucleic acid modification.

31. The method according to any one of claims 1-30 wherein said one or more immobilized nucleic acid molecules comprising a nucleic acid modification are phosphorylated prior to attaching to said adapter comprising a primer binding site.

32. The method according to any one of claims 1-31 wherein said nucleic acid modification comprises a DSB and said DSB is blunt ended before attaching to said adapter comprising a primer binding site.

33. The method according to any one of claims 1-32 wherein said adapter comprising said primer binding site further comprises a fluorescent moiety.

34. The method according to any one of claims 1-33 wherein the one or more immobilized nucleic acid molecules comprise a unique molecular identifier such as a barcode.

35. The method according to claim 34, wherein said barcode is a DNA or RNA barcode.

36. The method according to any one of claims 1-35 wherein said solid support is selected from a chip, an array a flow cell, a microwell, a microwell comprising an affinity treated surface and a bead, such as an immobilized affinity bead.

37. A method for detecting off-target activity of a targeted nuclease specific for a selected target sequence, the method comprising:
i. contacting a plurality of nucleic acid molecules immobilized on a solid support (immobilized nucleic acid molecules) with a complex comprising said targeted nuclease, thereby inducing one or more nucleic acid breaks;
ii. attaching an adapter comprising a primer binding site to one or more immobilized nucleic acid molecules comprising a nucleic acid break;
iii. sequencing at least part of said one or more immobilized nucleic acid molecules comprising a nucleic acid break using a primer specifically binding to said primer binding site;
iv. detecting the presence of breaks in a sequence of said one or more immobilized nucleic acid molecules other than in said selected target sequence.

38. A method for determining cleavage efficiency of a targeted nuclease specific for a selected target sequence, the method comprising:
i. contacting a plurality of nucleic acid molecules immobilized on a solid support (immobilized nucleic acid molecules) with a complex comprising said targeted nuclease, thereby inducing one or more nucleic acid breaks;
ii. attaching an adapter comprising a primer binding site to one or more immobilized nucleic acid molecules comprising a nucleic acid break;
iii. determining a proportion of said plurality of immobilized nucleic acid molecules comprising a nucleic acid break at said selected target sequence.

39. The method according to claim 38, wherein said determining is performed by:
sequencing at least part of said one or more immobilized nucleic acid molecules comprising a nucleic acid break using a primer specifically binding to said primer binding site, or
determining fluorescence intensity of said one or more immobilized nucleic acid molecules comprising said adapter which further comprises a fluorescent moiety.

40. The method according to claim 39, wherein said fluorescence intensity is determined cyclically, wherein each cycle comprises addition of said complex to said plurality of nucleic acid molecules followed by determining fluorescence intensity.

41. A method for selecting a guide RNA from a plurality of guide RNAs specific for a selected target sequence, the method comprising:
i. contacting a plurality of nucleic acid molecules immobilized on a solid support (immobilized nucleic acid molecules) with a plurality of RNA-guided nuclease complexes capable of inducing a nucleic acid break, said plurality of RNA-guided nuclease complexes comprising a plurality of different guide RNA's, thereby inducing one or more nucleic acid breaks;
ii. attaching an adapter comprising a primer binding site to said one or more immobilized nucleic acid molecules comprising a nucleic acid break;
iii. sequencing at least part of said one or more immobilized nucleic acid molecules comprising a nucleic acid break using a primer specifically binding to said primer binding site.
iv. selecting a guide RNA based on location and/or amount of said one or more breaks.

42. A method according to claim 41 wherein step iv comprises determining one or more locations in said one or more immobilized nucleic acid molecules comprising a break other than a location comprising said selected target sequence and selecting a guide RNA based on said one or more locations.

43. A method according to claim 41 or 42 wherein step iv comprises determining a number of sites in said one or more immobilized nucleic acid molecules comprising a break other than a site comprising said selected target sequence and selecting a guide RNA based on said number of sites.

44. A method according to any one of claims 37-43, wherein the method further comprises sequencing at least part of said one or more immobilized nucleic acid molecules prior to said contacting step i.

45. A method according to claim 44, wherein said method further comprises comparing the sequences obtained prior to and subsequent to said contacting step.

46. A method according to any one of claims 41-45 wherein said guide RNA is a single guide RNA (sgRNA).

47. The method according to any one of claims 37-46 wherein said nucleic acid is RNA or DNA.

48. The method according to any one of claims 37-47 wherein said nucleic acid is single stranded or double stranded.

49. The method according to any one of claims 37-48 wherein said nucleic acid molecules comprise genomic DNA (gDNA).

50. The method according to any one of claims 37-49 wherein said nucleic acid molecules comprises gDNA fragments.

51. The method according to claim 49 or 50 wherein said gDNA is obtained from a patient in need of genome editing.

52. The method according to any one of claims 37-51 wherein said break is a single strand break (SSB) or a double strand break (DSB).

53. The method according to any one of claims 37-52 wherein said complex comprising said targeted nuclease or said RNA-guided nuclease complexes comprises a ZFN, TALEN or CRISPR-Cas.

54. The method according to any one of claims 37-53 wherein said nuclease is selected from the group consisting of Cas9, Cpf1, C2c1, C2c2, C2c3, a group 29 nuclease, a group 30 nuclease and derivatives thereof.

55. The method according to any one of claims 37-54 wherein the method further comprises amplification of said plurality of nucleic acid molecules prior to said contacting with said complex or said plurality of complexes.

56. The method according to any one claims 37-55 wherein the method further comprises sequencing at least part of said plurality of immobilized nucleic acid molecules prior to said contacting with said complex or said plurality of complexes.

57. The method according to claim 56 wherein the method further comprises comparing the sequences obtained prior to and subsequent to said contacting with said complex or said plurality of complexes.

58. The method according to any one of claims 37-57 wherein the nucleic acid molecules are attached to said solid support via a chemical or protein linker.

59. The method according to claim any one of claims 37-58, wherein said solid support comprises a plurality of chemical or protein moieties and the method comprises, prior to said contacting step I, allowing one or more nucleic acid molecules flanked by a first and a second adapter, wherein at least one of said adapters comprises a chemical or biological moiety capable of binding to said chemical or biological moieties of said solid support to bind to said solid support.

60. The method according to any one of claims 37-59 wherein said method comprises prior to said contacting step i:
amplification of one or more nucleic acid molecules flanked by a first adapter comprising a first primer binding site and a second adapter comprising a second primer binding site in a droplet using primers specifically binding to said primer binding sites, wherein at least one of said primers comprises a chemical or biological moiety capable of binding to a solid support; and
allowing said amplified nucleic acid molecules to bind to said solid support.

61. The method according to claim 60, wherein said amplification comprises emulsion amplification.

62. The method according to any one of claims 37-57 wherein the method comprises allowing a plurality of nucleic acid molecules flanked by a first and a second adapter to hybridize to one of a plurality of first or second oligonucleotides that are immobilized on a solid support, whereby said first adapter comprises a sequence that is able to hybridize to said first immobilized oligonucleotides and said second adapter comprises a sequence that is complementary to a sequence that is able to hybridize to said immobilized second oligonucleotides.

63. The method according to any one of claims 37-62 wherein the method further comprises amplifying said plurality of immobilized nucleic acid molecules.

64. The method according claim 63 wherein said amplifying comprises bridge amplification.

65. The method according to claim 64 wherein the method comprises:
a) allowing one or more nucleic acid molecules flanked by a first and a second adapter to hybridize to one of said plurality of first or second immobilized oligonucleotides, whereby said first adapter comprises a sequence that is able to hybridize to said first immobilized oligonucleotides and said second adapter comprises a sequence that is complementary to a sequence that is able to hybridize to said immobilized second oligonucleotides;
and the bridge amplification comprises:
b) extending said first oligonucleotide with a polymerase whereby said single stranded nucleic acid molecules flanked by a first and a second adapter are used as a template;
c) removing said single stranded nucleic acid molecules flanked by a first and a second adapter used as a template resulting in a plurality of single stranded immobilized nucleic acid molecules;
d) hybridizing said one or more single stranded immobilized nucleic acid molecules to one of said plurality of immobilized second oligonucleotides;
e) extending said second oligonucleotide with a polymerase resulting in a plurality of double stranded immobilized nucleic acid molecules;
f) denaturing said plurality of double stranded immobilized nucleic acid molecules to produce a plurality of immobilized single stranded nucleic acid molecules; and
g) repeating steps d-f at least once.

66. The method according to any one of claims 37-65 wherein said immobilized nucleic acid molecules are unphosphorylated.

67. The method according to any one of claims 37-64 wherein said immobilized nucleic acid molecules are treated with phosphatase prior to said contacting with said complex or said complexes.

68. The method according to any one of claims 37-67 wherein said immobilized cleaved nucleic acid molecules are phosphorylated prior to attaching to said adapter comprising a primer binding site.

69. The method according to any one of claims 37-68 wherein said break is a DSB and said DSB is blunt ended before attaching to said adapter comprising a primer binding site.

70. The method according to any one of claims 37-69 wherein said immobilized nucleic acid molecules comprise a unique molecular identifier, such as a barcode.

71. The method according to claim 70, wherein said barcode is a DNA or RNA barcode.

72. The method according to any one of claims 37-71 wherein said solid support is selected from a chip, an array, a flow cell, a microwell, a microwell comprising an affinity treated surface and a bead, such as an immobilized affinity bead.

73. A kit of parts comprising the components for executing the method according to any of claims 1 to 72.

74. A kit of parts comprising a solid support comprising one or more nucleic acid molecules immobilized thereon and an agent capable of inducing a nucleic acid modification.

75. The kit of parts according to claim 74 wherein said nucleic acid modification is selected from the group consisting of a mutation, a deletion, an insertion, a replacement, a ligation, a digestion, a break and a recombination.

76. The kit of parts according to claim 74 or 75 wherein said agent is selected from the group consisting of a chemical agent, a (viral) integrase, a recombinases, a transposase, an argonaute, a cytidine deaminase, a retron and a group II intron.

77. The kit of parts according to claim 74 or 75 wherein agent comprises a targeted nuclease complex.

78. The kit of parts according to claim 77 wherein said targeted nuclease complex comprises a ZFN, TALEN or CRISPR-Cas.

79. A kit of parts comprising a targeted nuclease and a solid support.

80. The kit of parts according to claim 79, wherein said solid support comprises a plurality of first and second oligonucleotides immobilized thereto.

81. The kit of parts according to claim 81 further comprising a first adapter comprising a sequence that is able to hybridize to said first immobilized oligonucleotides and a second adapter comprising a sequence that is complementary to a sequence that is able to hybridize to said immobilized second oligonucleotides.

82. The kit of parts according to claim 79, wherein said solid support comprises a plurality of chemical or protein linkers.

83. The kit of parts according to claim 82 further comprising a first adapter comprising a first primer binding site and a second adapter comprising a second primer binding site, wherein at least one of said adapters comprises a chemical or biological moiety capable of binding to said chemical or protein linkers.

84. The kit of parts according to any one of claims 79-83 further comprising one or more nucleic acid molecules.

85. The kit of parts according to any one of claims 73-78 and 84 wherein said nucleic acid is RNA or DNA.

86. The kit of parts according to any one of claim 73-78, 84 or 85 wherein said nucleic acid is single stranded or double stranded.

87. The kit of parts according to any one of claim 73-78 or 84-86 wherein said one or more nucleic acid molecules comprise genomic DNA (gDNA).

88. The kit of parts according to any one of claim 73-78 or 84-87 wherein said one or more nucleic acid molecules comprise gDNA fragments.

89. The kit of parts according to any one of claim 73-78 or 84-88 wherein said nucleic acid molecule are flanked by a first and a second adapter, whereby said first adapter comprises a sequence that is able to hybridize to said first immobilized oligonucleotides and said second adapter comprises a sequence that is complementary to a sequence that is able to hybridize to said immobilized second oligonucleotides.

90. The kit of parts according to any one of claims 77-89 wherein said nuclease is selected from the group consisting of Cas9, Cpf1, C2c1, C2c2, C2c3, a group 29 nuclease, a group 30 nuclease and derivatives thereof

91. The kit of parts according to any one of claims 73-90 further comprising one or more components selected from the group consisting of a DNA or RNA polymerase, a restriction enzyme, a ligase, an exonuclease, a mixture of nucleotides and labelled nucleotides.

92. The kit of parts according to claim 91, wherein said labelled nucleotides comprises adenine, guanine, cytosine, thymine and/or uracil, whereby each nucleotide is labelled with a different fluorescent moiety.

93. The kit of parts according to claim 91 or 92 wherein the nucleotides or labelled nucleotides are modified nucleotides, such as dideoxy nucleotides or nucleotides comprising a phosphorothiate linkage.

94. The kit of parts according to any one of claims 73-93 wherein said solid support is selected from a chip, an array, a flow cell, a microwell, a microwell comprising an affinity treated surface and a bead, such as an immobilized affinity bead.

95. A method for enrichment of one or more nucleic acid molecules wherein a nucleic acid modification is made, the method comprising:
i. contacting a plurality of nucleic acid molecules with an agent capable of inducing a nucleic acid modification, wherein said nucleic acid molecules are flanked by a first adapter comprising a first primer binding site and a ligation-blocking moiety and a second adapter comprising a second primer binding site and a ligation-blocking moiety, resulting in one or more modified nucleic acid molecules; and
ii. amplifying said one or more modified nucleic acid molecules comprising said adapter using a primer that binds to said first or second primer binding site and a primer that binds to a third primer binding site,
wherein said method comprises attaching an adapter comprising said third primer binding site to said one or more modified nucleic acid molecules following said contacting step and prior to amplifying in step ii, or
wherein said modification comprises insertion of an adapter comprising said third primer binding site.

96. The method according to claim 95, wherein said first and second primer binding sites are identical.

97. The method according to claim 95, wherein said first and second primer binding sites are different.

98. The method according to claim 97 wherein said adapter comprising a third primer binding site further comprises a fourth primer binding site.

99. The method according to claim 98, wherein said fourth primer binding site is identical to said first or second primer binding site.

100. The method according to any one of claims 95-99 wherein said primer that binds to said third primer binding site comprises a fifth primer binding site.

101. The method according to claim 100, wherein said fifth primer binding site is identical to said first or second primer binding site.

102. The method according to any one of claims 97-101 wherein the method further comprises amplifying one or more nucleic acid molecules that have not been modified using said primers that bind to said first and second primer binding site.

103. The method according to any one of claims 95-102, wherein said plurality of nucleic acid molecules is a plurality of RNA molecules, and said amplifying comprises reverse transcription using a primer that binds to said third primer binding site.

104. The method according to any one of claims 95-102 wherein said plurality of nucleic acid molecules is a plurality of DNA molecules, said adapter comprising a third primer binding site further comprises a DNA-dependent RNA polymerase promotor and said method further comprises, prior to said amplifying:
performing transcription of said one or more cleaved DNA molecules using said DNA-dependent RNA polymerase, resulting in one or more transcribed RNA molecules; and
digesting DNA molecules,
and wherein said amplifying comprises amplifying said one or more transcribed RNA molecules using primers that bind to said first or second primer binding site and to said third primer binding site.

105. The method according to claim 104, wherein said amplifying comprises reverse transcription of said RNA molecules.

106. The method according to claim 104 or 105 wherein said digesting is performed using a DNAse.

107. The method according to any one of claims 95-106 wherein said primer that binds to said third primer binding site is an indexing primer.

108. A method for detecting a nucleic acid modification, comprising:
enriching one or more nucleic acid molecules wherein a nucleic acid modification is induced with a method according to any one of claims 95-107; and
sequencing at least part of said amplified modified nucleic acid molecules.

109. A method for detecting a nucleic acid modification, comprising:
enriching one or more nucleic acid molecules wherein a nucleic acid modification is induced with a method according to any one of claims 95-107;
sequencing at least part of said amplified modified nucleic acid molecules; and
sequencing at least part of said amplified nucleic acid molecules that have not been modified.

110. The method according to any one of claims 95-109 wherein said plurality of nucleic acid molecules, said adapter comprising a first primer binding site, said adapter comprising a second primer binding site and said adapter comprising a third primer binding site are double stranded.

111. The method according to any one of claims 95-110 wherein said nucleic acid modification is selected from the group consisting of an insertion, a replacement, a strand break and a recombination.

112. The method according to claim 111 wherein said break is a double stranded break (DSB), a nick or a single stranded break (SSB).

113. The method according to any one of claims 95-112 wherein said nucleic acid is double stranded, said nucleic acid modification is a nick and said method further comprises contacting said modified nucleic acid molecules with an S1 nuclease subsequent to said contacting with an agent capable of inducing a nick.

114. The method according to any one of claims 95-113 wherein said break is a double stranded break (DSB) and wherein cleaved nucleic acid molecules are blunt ended before ligating to said adapter comprising a third primer binding site.

115. The method according to any one of claims 95-114 wherein said adapter comprising a third primer binding site further comprises an adenine-tail.

116. The method according to any one of claims 95-115 wherein said ligation-blocking moiety comprises a dideoxynucleotide.

117. The method according to any one of claims 95-116 wherein said adapter comprising a first primer binding site or said adapter comprising a second primer binding site further comprise a unique molecular identifier such as a barcode.

118. The method according to any one of claims 95-117 wherein said agent comprises a nuclease.

119. The method according to any one of claims 95-118 wherein said agent comprises a targeted nuclease complex or a plurality of targeted nuclease complexes.

120. The method according to claim 119 wherein said targeted nuclease complex or complexes comprises a ZFN, TALEN or CRISPR-Cas.

121. The method according to claim 119, wherein said plurality of targeted nuclease complexes comprises a plurality of guide RNA's.

122. The method according to any one of claims 118-121 wherein said nuclease is selected from the group consisting of Cas9, Cpf1, C2c1, C2c2, C2c3, a group 29 nuclease, a group 30 nuclease and derivatives thereof.

123. The method according to any one of claims 95-122 wherein said one or more nucleic acid molecules comprise genomic DNA (gDNA).

124. The method according to any one of claims 95-123 wherein said one or more nucleic acid molecules comprise gDNA fragments.

125. The method according to claim 123 or 124 wherein said gDNA is obtained from a patient in need of genome editing.

126. A method for detecting off-target activity of a targeted nuclease specific for a selected target sequence, comprising:
enriching one or more nucleic acid molecules wherein a nucleic acid break is induced with a method according to any one of claims 95-107 and 110-125, wherein said agent comprises a targeted nuclease complex and
detecting the presence of breaks in a sequence of said one or more nucleic acid molecules other than in said selected target sequence.

127. A method for determining cleavage efficiency of a targeted nuclease specific for a selected target sequence, comprising:
enriching one or more nucleic acid molecules wherein a nucleic acid break is induced with a method according to any one of claims 95-107 and 110-125, wherein said agent comprises a targeted nuclease complex and
determining a proportion of said plurality of nucleic acid molecules comprising a nucleic acid break at said selected target sequence.

128. A method for selecting a guide RNA from a plurality of guide RNAs specific for a selected target sequence, the method comprising:
enriching one or more nucleic acid molecules wherein one or more nucleic acid breaks are made with a method according to any one of claims 95-107 and 110-125, whereby said plurality of nucleic acid molecules is contacted with a plurality of RNA-guided nuclease complexes capable of inducing a nucleic acid break; and
selecting a guide RNA based on location and/or amount of said nucleic acid breaks.

129. The method according to claim 128 wherein said selecting comprises determining one or more locations in said one or more nucleic acid molecules comprising a break other than a location comprising said selected target sequence and selecting a guide RNA based on said one or more locations.

130. The method according to claim 128 or 129 wherein said selecting comprises determining a number of sites in said one or more nucleic acid molecules comprising a break other than a site comprising said selected target sequence and selecting a guide RNA based on said number of sites.

131. A method for detecting a nucleic acid break, comprising:
i. contacting a plurality of nucleic acid molecules flanked by adapters comprising a ligation-blocking moiety with an agent capable of inducing a nucleic acid break, resulting in one or more cleaved nucleic acid molecules;
ii. attaching an adapter comprising a primer binding site to said one or more cleaved nucleic acid molecules;
iii. sequencing at least part of said one or more cleaved nucleic acid molecules using a primer specifically binding to said primer binding site, said part comprising said nucleic acid modification.
